(19) United States
(12) Patent Application Publication
(10) Pub. No.: US 2011/0150820 A1
(43) Pub. Date: Jun. 23, 2011

(54) METHODS FOR COVALENTLY ATTACHING A POLYMER TO A METHIONINE RESIDUE IN PROTEINS AND PEPTIDES

(75) Inventors: Joel M. Van Gelder, Jerusalem (IL); Andrei Serban, Rishon-LeZion (IL); Susanna Tchilibon, Jerusalem (IL); Daphna Miron, Rechovot (IL)

(73) Assignee: Insight Biopharmaceuticals Ltd., Rehovot (IL)

(21) Appl. No.: 13/060,439

(22) PCT Filed: Aug. 27, 2009
(86) PCT No.: PCT/IL09/00832

§ 371 Date: Feb. 24, 2011

Related U.S. Application Data
(60) Provisional application No. 61/136,332, filed on Aug. 28, 2008.

(51) Int. Cl. A61K 47/48; C08G 65/48; A61P 37/04

(52) U.S. Cl. 424/78.17; 525/54.1; 525/418

(57) ABSTRACT

Conjugates of polypeptides and a polymeric moiety such as PEG covalently attached to the sulfur atom of a methionine side chain are disclosed. Processes of preparing such conjugates, including intermediates and reagents utilized therefore are also disclosed. Further disclosed are therapeutic uses of these conjugates.

### FIELD AND BACKGROUND OF THE INVENTION

The present invention relates to methods of attaching a polymeric moiety to active proteins and peptides, and, more particularly, to methods of attaching polyethylene glycol and/or related polymeric moieties to therapeutically active proteins and peptides so as to improve the pharmacological performance thereof.

Most therapeutic proteins are short-lived and have often a short circulatory half-life in vivo, and therefore the pharmaceutical uses thereof are critically limited by their in vivo and ex vivo instability and by their poor pharmacokinetics. This is particularly valid for non-glycosylated proteins of a molecular mass less than 50 kDa.

The short lifetime of proteins in vivo is attributed to several factors, including glomerular filtration in the kidney, and proteolysis in the stomach, bloodstream and liver. Moreover, proteins are typically characterized by poor absorption after oral ingestion, in particular due to their relatively high molecular mass and/or the lack of specific transport systems. Proteins are easily degraded in oxidative and acidic environments and therefore typically require intravenous administration (so as to avoid, e.g., degradation in the gastrointestinal tract).

Considering that therapeutic proteins are not absorbed orally, prolonged maintenance of these therapeutically active drugs in the circulation is still a considerable challenge of great clinical importance, since proteins are further broken down in the blood system and liver by proteolytic enzymes and are rapidly cleared from the circulation, and further have the tendency to evoke an immunological response particularly when their sequence is not recognized by the host's immune system.

In addition, proteins are heat and humidity sensitive and therefore their maintenance requires costly care, complex and inconvenient modes of administration, and high-cost of production and maintenance. The above disadvantages impede the use of proteins as efficient drugs and stimulate the quest for means to alter some of the characteristics of proteins so as to bestow robustness and stability thereto.

It has been shown that when a suitable polymer is conjugated to a protein, the conjugate polymer endows the aqueous solubility to the protein, masks potential epitope and proteolysis sites on the protein, and increases the molecular weight and volume thereof. The attachment of a suitable polymer to a protein has been shown to be efficacious in reducing the rate of clearance of the therapeutic protein drug from the physiological system, reducing renal clearance, reducing proteolysis, reducing antigenicity, and increasing water solubility, while retaining a substantial proportion of the biological activity of the protein.

Among all the polymers studied so far, polyethylene glycol (PEG) emerged as an optimal candidate for being conjugated to proteins and polypeptides and modification thereof due to its uncommon properties, discussed hereinbelow, which are conferred on the protein-PEG conjugate. This promising technology for improving pharmaceutical and clinical properties of therapeutic proteins is known as PEGylation. Thus, the term PEGylation defines the modification of a protein, peptide or non-peptide molecule by the chemical linking, via covalent attachment, of one or more poly(ethylene glycol) (PEG) chains thereto.

The optimization of both the polymerization procedure and purification process allowed the development of PEGs with low polydispersity, spanning from 1.01, for PEG below 5 kDa molecular weight, to 1.1 for PEG with molecular weight as high as 50 kDa.

Alcohol dehydrogenase can degrade low molecular weight PEGs, and chain cleavage can be catalyzed by P450 microsomal enzymes. PEG has been used for several years as an excipient in foods, cosmetics and pharmaceuticals and is considered non-toxic and therefore FDA-approved for human use. The first common positive effect of PEGylation is an extended half-life in the bloodstream due to reduced kidney filtration and clearance. Consequently, a PEGylated therapeutic protein requires reduced frequency of administration with respect to a non-PEGylated protein. Moreover, PEG is a highly suitable polymer for protein conjugation in the context of therapeutic purposes due to the lack of immunogenicity and antigenicity on its part. PEG has been shown to decrease immunogenicity of the protein presumably by protecting the protein from being recognized as foreign antigen by the immune system owing to coverage or blockage of critical protein's sites which are recognized epitopes, and by masking specific sequence regions which are degraded by proteolytic enzymes.

Site-preferential PEGylation of proteins and polypeptides can be achieved by exploiting the different surface accessibility of the protein's amino groups, as demonstrated for a truncated form of growth hormone-releasing hormone (hGRF1-29), thiol groups, as demonstrated for interferon beta (IFN-beta), carboxyl groups as demonstrated with truncated thrombomodulin mutant, hydroxyl groups as demonstrated with epidermal growth factor, and also demonstrated with some protein's guanidine groups. However, site-preferred method is often inapplicable, or limited by low yield, in cases where, for example, the PEGylation is directed at a buried or less accessible amino acid, and particularly when a high molecular weight PEG is required. Thus, other approaches are desired, and some have been designed.

While PEGylation chemistry of proteins through reactions with a variety of reactive amino acids has been thoroughly investigated, none of the presently known methodologies for protein PEGylation teach the attachment of PEG to the sulfur atom in the side chain of the amino acid residue methionine, albeit chemical modification thereof is known for many years [Glazer, A N, Annu. Rev. Biochem., 39, 101, 1970]. This particular amino acid is the second least frequent amino acid in expressed genes having the single tri-nucleotide codon AUG, and an observed frequency of 1.8% in vertebrates. Having a unique chemical feature among all amino acid side chains, thiomethoxy, and being relatively infrequent, this amino acid residue presents a lucrative opportunity for site-directed and discriminating PEGylation of proteins.

An example of a naturally occurring modification of methionine side-chain is S-methylmethionine, also known as “vitamin U”. This compound is produced in plants by the enzyme methionine S-methyltransferase and was identified as an anti-ulcer agent [McRolly, R A. et al., J. Am. Chem. Soc., 76, 115, 1954] and as potential agent against nephritic hyperlipidemia [Seri, K. et al., Arzneim. Forsch., 29, 1517, 1979]. In addition, nature also makes extensive use of the S-adenosylmethionine sulfonium salt as a biological methylating agent [Grillo, M A and Colombatto, S, Amino Acids, 28, 357, 2005].

Introduction of a carboxymethyl group (carboxymethylation) into various side chains of amino acids such as cysteine, lysine, histidine and methionine is currently used in chemical modification reactions of proteins [Gurd, F R N, Methods Enzymol., 1, 532, 1967]. The alkylating agents used in this type of reaction are typically haloacetates or haloacetamides. This modification reaction is typically most effective near or above the pH corresponding to the pKa of the individual amino acid, namely for histidine at or above pH=5 and for lysine at or above pH=7. Only in the case of methionine, the thioether is available for reaction over the entire pH range unless the side chain is in a masked state. Therefore, accessible methionine side-chain residues may be selectively alkylated at an acidic pH, namely pH=4. This reaction has been used for selective chemical modification of methionines in several proteins [see, for example, Gundlach, H G. et al., J. Biol. Chem., 234, 1754, 1959; Holeysovsky, V. and Lazdunski, M., Biochim. Biophys. Acta, 154, 457, 1968; Colma, R F., J. Biol. Chem., 243, 2454, 1968; Naider, F. et al., Biochemistry, 11, 3202, 1972; Cheng, K W., Biochem. J., 159, 79, 1976; Czupryn, M J. et al., J. Biol. Chem., 270, 978, 1995; and Blouin, C. et al., Biochem. Cell Biol., 80, 197, 2002].

The carboxymethylation reaction has also been used for selective binding of methionyl-containing peptides and proteins to polymeric resins having a haloacetamide linker [Shechter, Y. et al., Biochemistry, 16, 1424, 1977]. Other alkylation agents, such as benzyl bromide, have also been used for selective modification of methionines in proteins such as fumarase [Rogers, G A. et al., J. Biol. Chem., 251, 5711, 1976], CAMP factor and calmodulin [Lang, S. et al., Anal. Biochem., 359, 253, 2006].

Methionine residues have also been labeled by alkylation with dansylaziridine, as in the case of the calcium-binding component of troponin [Grabarek, Z. et al., J. Biol. Chem., 258, 14098, 1983] and with epoxides under acidic conditions [Alferiev, I S et al., Biomaterials, 22, 2501, 2001].

### SUMMARY OF THE INVENTION

While chemical modification of some proteins at the side-chain of methionine has been reported to some extent, proteins which have been modified, at the side-chain of a methionine residue found in their amino-acid sequence, by moieties such as PEG, which improve the pharmacokinetic profile and hence the therapeutic activities of the proteins, have not been reported hitherto.

The present inventors have now designed and successfully practiced a novel method of PEGylating proteins via a side chain of a methionine residue within the protein.

Thus, according to one aspect of embodiments of the present invention there is provided a conjugate which includes (a) a polypeptide having one or more methionine residue(s), each methionine residue having a methylsulfanyl-ethyl side-chain; and (b) one or more polymer moieties being covalently attached to a sulfur atom of the methylsulfanyl-ethyl side-chain of one or more the methionine residue(s).

According to some embodiments of the invention the polymer moiety is covalently attached to the sulfur atom via a linking moiety.

According to some embodiments of the invention the linking moiety comprises one or more residues of a reactive moiety, the reactive moiety being selected capable of reacting with the sulfur atom of the methylsulfanyl-ethyl side-chain.

According to some embodiments of the invention the reactive moiety is selected from the group consisting of amine, carboxyl, amide, acetamide, 2-halo-acetamide, (4-halomethyl)-benzamide, benzyl-halide, hydrazine, hydrazide, acetohydrazide, alkyl, haloalkyl, alkyl sulfonylhalide, alkyl tosylate, alkyl triflate, allyl, haloallyl, allyl sulfonylhalide, allyl tosylate, allyl triflate, aryl, haloaryl heteroaryl, 4-(halomethyl)benzyl, benzyl and halobenzyl, and any combination thereof.

According to some embodiments of the invention the reactive moiety comprises a leaving group such as, but not limited to, halide, acetate, tosylate, triflate, sulfonate, azide, hydroxy, thiohydroxy, alkoxy, cyanate, thiocyanate, nitro and cyano.

According to some embodiments of the invention the linking moiety further comprises a spacer. According to some embodiments of the invention the spacer is selected from the group consisting of a linear or branched, saturated or unsaturated, substituted or unsubstituted alkylene chain having 1-30 carbon atoms, and a linear or branched, saturated or unsaturated, substituted or unsubstituted alkylene chain having 1-30 carbon atoms interrupted by one or more heteroatom, whereby the one or more heteroatom is selected from the group consisting of oxygen, sulfur, nitrogen, phosphor and/or silicon.

According to some embodiments of the invention the polypeptide is selected from the group consisting of an interferon, a cytokine, a hormone, a growth factor, an enzyme, a blood protein (factor), an antibody, an antigen, a viral protein, a fusion protein, and any part or segment thereof.

Representative examples include adalimumab, adenosine deaminase, agalsidase-beta, alglucosidase-alpha, alpha-galactosidase, asparaginase, B-deleted domainFactor VIII, bone morphogenetic protein-2 (BMP-2), bone morphogenetic protein-7 (BMP-7), brain-derived neurotrophic factor (BDNF), cetuximab, chorionic gonadotropin (CG), dornase-alpha, erythropoietin (EPO), etanercept, Factor IX, Factor VIIa, Factor VIII, follicle stimulating hormone (FSH), galsulfase, glial cell line derived neurotrophic factor (GDNF), glucagon, granulocyte colony stimulating factor (G-CSF), granulocyte-macrophage colony stimulating factor (GM-CSF), growth hormone (GH), hemoglobin, heparanase, hyaluronidase, imiglucerase, infliximab, insulin-like growth factor-1 (IGF-1), interferon-alpha-2a (IFN alpha-2a), interferon-alpha-2b (IFN alpha-2b), interferon-beta-1a (IFN beta-1a), interferon-beta-1b (IFN beta-1b), interferon-gamma-1b (IFN gamma-1b), interleukin (IL-11), interleukin (IL-2), interleukin-1 (IL-1) receptor antagonist, interleukin-1 receptor antagonist (IL-1ra), keratinocyte growth factor (KGF), laronidase, luteinizing hormone (LH), megakaryocyte growth differentiation factor (MGDF), obesity protein (OB protein or leptin), osteoprotegerin (OPG), parathyroid hormone (PTH or 1-34 segment or PTH 1-34), palivizumab, platelet-derived growth factor (PDGF), Protein C, rituximab, stem cell factor (SCF), streptokinase, thrombin, thrombopoietin (TPO), thyrotropin (TSH), tissue plasminogen activator (tPA), trastuzumab, tumor necrosis factor binding protein (TNFbp), tumor necrosis factor-alpha (TNF-alpha) and urate-oxidase.

According to some embodiments of the invention the polymer moiety is selected from the group consisting of a polyalkylene glycol, a polyethylene glycol (PEG), a poly(lactic acid) (PLA), a polyester, a polyglycolide (PGA), a polycaprolactone (PCL), a polyamide, a polymethacrylamide, a polyvinyl alcohol, a polycarboxylate, a polyvinyl pyrrolidinone, a dextran, a cellulose, a chitosan, a hydroxyethyl starch (HES), polyamino acids such as polyglutamic acid and polyglycine and any copolymer thereof.

According to some embodiments of the invention the polymer moiety has an average molecular weight that ranges from about 1 kDa to about 100 kDa.

According to some embodiments of the invention the polymer moiety is a polyethylene glycol (PEG). According to some embodiments of the invention the polyethylene glycol has an average molecular weight that ranges from 4 kDa to 40 kDa.

According to some embodiments of the invention the polypeptide has a characterizing biological activity and the conjugate has a biological activity of a kind characterizing the polypeptide.

According to some embodiments of the invention the conjugate is soluble in saline.

According to some embodiments of the invention the polypeptide has a characterizing three dimensional structure and the conjugate has a three dimensional structure of a kind characterizing the polypeptide.

According to some embodiments of the invention the polypeptide has a characterizing three dimensional structure in which one or more methionine residues is positioned at an outer surface of the three dimensional structure.

According to some embodiments of the invention the polypeptide has a characterizing three dimensional structure, in which one or more methionine residues is positioned at an outer surface of the three dimensional structure, and the polymer forms a stable chemical interaction with the outer surface at physiological conditions.

According to some embodiments of the invention the polypeptide has a characterizing half life under physiological conditions and the conjugate has a greater half life under the physiological conditions.

According to another aspect of embodiments of the present invention there is provided a compound which comprises (a) a polypeptide having one or more methionine residues, each methionine residue having a methylsulfanyl-ethyl side-chain; and (b) one or more modifying moieties, each comprising a residue of a first reactive moiety and a second reactive moiety. The modifying moiety is covalently attached to a sulfur atom of the methylsulfanyl-ethyl side-chain of a methionine residue via the residue of the first reactive moiety, and the polypeptide is selected from the group consisting of adalimumab, adenosine deaminase, agalsidase-beta, alglucosidase-alpha, alpha-galactosidase, asparaginase, B-deleted domain Factor VIII, bone morphogenetic protein-2 (BMP-2), bone morphogenetic protein-7 (BMP-7), brain-derived neurotrophic factor (BDNF), cetuximab, chorionic gonadotropin (CG), dornase-alpha, erythropoietin (EPO), etanercept, Factor IX, Factor VIIa, Factor VIII, follicle stimulating hormone (FSH), galsulfase, glial cell line derived neurotrophic factor (GDNF), glucagon, granulocyte colony stimulating factor (G-CSF), granulocyte-macrophage colony stimulating factor (GM-CSF), growth hormone (GH), hemoglobin, heparanase, hyaluronidase, imiglucerase, infliximab, insulin-like growth factor-1 (IGF-1), interferon-alpha-2a (IFN alpha-2a), interferon-alpha-2b (IFN alpha-2b), interferon-beta-1a (IFN beta-1a), interferon-beta-1b (IFN beta-1b), interferon-gamma-1b (IFN gamma-1b), interleukin (IL-11), interleukin (IL-2), interleukin-1 (IL-1) receptor antagonist, interleukin-1 receptor antagonist (IL-1ra), keratinocyte growth factor (KGF), laronidase, luteinizing hormone (LH), megakaryocyte growth differentiation factor (MGDF), obesity protein (OB protein or leptin), osteoprotegerin (OPG), parathyroid hormone (PTH or 1-34 segment or PTH 1-34), palivizumab, platelet-derived growth factor (PDGF), Protein C, rituximab, stem cell factor (SCF), streptokinase, thrombin, thrombopoietin (TPO), thyrotropin (TSH), tissue plasminogen activator (tPA), trastuzumab, tumor necrosis factor binding protein (TNFbp), tumor necrosis factor-alpha (TNF-alpha) and urate-oxidase.

According to some embodiments of the invention the first reactive moiety is selected capable of reacting with the sulfur atom, and can be selected from the group consisting of amine, carboxyl, amide, acetamide, 2-halo-acetamide, (4-halomethyl)-benzamide, benzyl-halide, hydrazine, hydrazide, acetohydrazide, alkyl, haloalkyl, alkyl sulfonylhalide, alkyl tosylate, alkyl triflate, allyl, haloallyl, allyl sulfonylhalide, allyl tosylate, allyl triflate, aryl, haloaryl heteroaryl, 4-(halomethyl)benzyl, benzyl and halobenzyl, and any combination thereof.

According to some embodiments of the invention the first reactive moiety comprises one or more leaving groups such as, but not limited to, halide, acetate, tosylate, triflate, sulfonate, azide, hydroxy, thiohydroxy, alkoxy, cyanate, thiocyanate, nitro and cyano.

According to some embodiments of the invention the second reactive moiety is selected from the group consisting of amine, carboxyl, amide, hydrazine, hydrazide, thiol, hydroxyl and hydroxylamine, and any combination thereof.

According to some embodiments of the invention the modifying moiety further comprises a spacer connecting the residue of the first reactive moiety and the second reactive moiety.

According to some embodiments of the invention the spacer is selected from the group consisting of methane-di-yl, ethane 1-yl-2-yl, propane 1-yl-3-yl, butane 1-yl-4-yl, 1,4-benzene-diyl and 1,10-biphenyl-diyl.

According to some embodiments of the invention the compound further comprises a labeling moiety being covalently attached to the modifying moiety.

According to some embodiments of the invention the labeling moiety is selected from the group consisting of a fluorescent moiety, a radioactive moiety, a magnetic moiety, a chromophore, a phosphorescent moiety and a heavy metal cluster, and any combination thereof.

According to another aspect of embodiments of the present invention there is provided a conjugate comprising interferon-beta-1b and 30 kDa methoxy polyethylene glycol N-ethyl-acetamide, being attached therebetween via a sulfur atom of a methionine residue of the interferon-beta-1b, and having a formula:

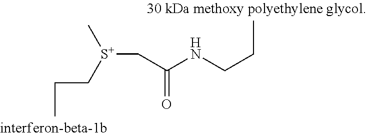

According to another aspect of embodiments of the present invention there is provided a conjugate comprising interferon-beta-1b and 30 kDa methoxy polyethylene glycol N-ethyl-(4-bromomethyl)-benzamide, being attached therebetween via a sulfur atom of a methionine residue of the interferon-beta-1b, and having a formula:

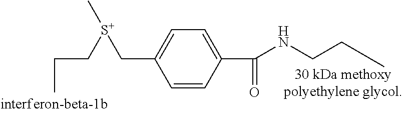

According to another aspect of embodiments of the present invention there is provided a conjugate comprising interferon-beta-1a and 30 kDa methoxy polyethylene glycol N-ethyl-acetamide, being attached therebetween via a sulfur atom of a methionine residue of the interferon-beta-1a, and having a formula:

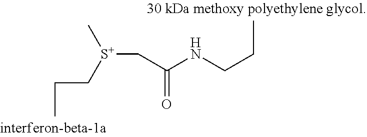

According to another aspect of embodiments of the present invention there is provided a conjugate comprising interferon-alpha-2a and 30 kDa methoxy polyethylene glycol N-ethyl-acetamide, being attached therebetween via a sulfur atom of a methionine residue of the interferon-alpha-2a, and having a formula:

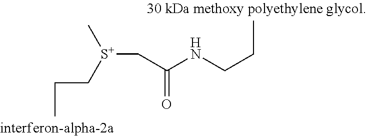

According to another aspect of embodiments of the present invention there is provided a conjugate comprising interferon-alpha-2a and 30 kDa methoxy polyethylene glycol N-ethyl-(4-bromomethyl)-benzamide, being attached therebetween via a sulfur atom of a methionine residue of the interferon-alpha-2a, and having a formula:

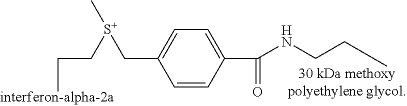

According to another aspect of embodiments of the present invention there is provided a conjugate comprising erythropoietin and 30 kDa methoxy polyethylene glycol N-ethyl-(4-bromomethyl)-benzamide, being attached therebetween via a sulfur atom of a methionine residue of the erythropoietin, and having a formula:

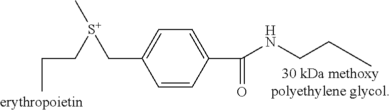

According to another aspect of embodiments of the present invention there is provided a conjugate comprising granulocyte colony-stimulating factor (G-CSF) and 30 kDa methoxy polyethylene glycol N-ethyl-(4-bromomethyl)-benzamide, being attached therebetween via a sulfur atom of a methionine residue of the G-CSF, and having a formula:

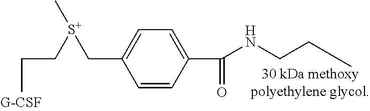

According to another aspect of embodiments of the present invention there is provided a conjugate comprising human growth hormone (h-GH) and 30 kDa methoxy polyethylene glycol N-ethyl-(4-bromomethyl)-benzamide, being attached therebetween via a sulfur atom of a methionine residue of the h-GH, and having a formula:

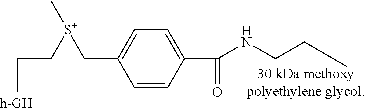

According to another aspect of embodiments of the present invention there is provided a conjugate comprising human follicle stimulating hormone (h-FSH) and 30 kDa methoxy polyethylene glycol N-ethyl-(4-bromomethyl)-benzamide, being attached therebetween via a sulfur atom of a methionine residue of the h-FSH, and having a formula:

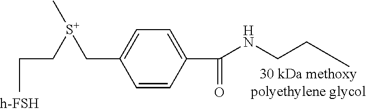

According to yet another aspect of embodiments of the present invention there is provided a process of preparing a conjugate as described herein, which comprises a polypeptide having one or more methionine residues and one or more polymer moieties attached to a sulfur atom of a methylsulfanyl-ethyl side-chain of the methionine residues, the process is effected by reacting the polypeptide with a polymer having one or more reactive moieties attached thereto under acidic conditions ranging from about pH of 2 to pH of 5.

According to still another aspect of embodiments of the present invention there is provided a process of preparing the modified polypeptide compound described herein, the process is effected by reacting the polypeptide with a modifying moiety having a first reactive moiety and a second reactive moiety under acidic conditions ranging from pH 2 to pH 5, whereby the first and second reactive moieties are selected such that a covalent bond is formed between the first reactive group and the sulfur atom.

According to still another aspect of embodiments of the present invention there is provided a process of preparing a conjugate which includes (a) a polypeptide having one or more methionine residues; and (b) one or more polymer moieties attached to a sulfur atom of a methylsulfanyl-ethyl side-chain of the methionine residues; the process is effected by reacting the polypeptide with a modifying moiety having a first reactive moiety and a second reactive moiety under acidic conditions ranging from pH 2 to pH 5, the first and second reactive moieties are selected such that a covalent bond is formed between the first reactive moiety and the sulfur atom, to thereby obtain a polypeptide having the one or more modifying moiety attached thereto; and reacting the polypeptide having the one or more modifying moieties attached thereto with a polymer having a third reactive moiety, the third reactive moiety is selected capable of reacting with the second reactive moiety in the modifying moiety.

According to some embodiments of the invention the second and the third reactive moieties are each independently selected from the group consisting of amine, carboxyl, amide, hydrazine, hydrazide, thiol, hydroxyl and hydroxylamine, and any combination thereof.

According to an additional aspect of embodiments of the present invention there is provided a pharmaceutical composition which comprises, as an active ingredient, a conjugate as described herein.

According to still an additional aspect of embodiments of the present invention there is provided a use of a conjugate as described herein in the manufacture of a medicament.

According to still an additional aspect of embodiments of the present invention there is provided a method of treating a medical condition treatable by a polypeptide having at least one methionine residue, the method is effected by administering to a subject in need thereof an therapeutically effective amount of a conjugate as described herein.

According to a further aspect of embodiments of the present invention there is provided a compound which comprises a polyalkylene glycol moiety and a benzyl halide moiety being covalently linked therebetween via a linking moiety.

According to some embodiments of the invention the polyalkylene glycol moiety is a polyethylene glycol (PEG).

According to some embodiments of the invention the polyethylene glycol has an average molecular weight that ranges from 4 kDa to 40 kDa.

According to some embodiments of the invention the linking moiety is selected from the group consisting of amine, alkyl, aryl, heteroaryl, carboxyl, amide, hydrazine, hydrazide and any combination thereof.

According to some embodiments of the invention the polyalkylene glycol-benzyl halide compound further includes a spacer linking the benzyl halide moiety and the linking moiety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents a reverse-phase HPLC (RP-HPLC) chromatogram of the PEGylation reaction products of recombinant human interferon-beta-1b and 30 kDa methoxy polyethylene glycol N-ethyl-2-iodo-acetamide, obtained using a photo-diode array set at 280 nm, an injection volume of 40 μl, total run-time of 70 minutes and 0.2% TFA in water/acetonitrile as a mobile phase, showing a peak having a retention time of 29.17 minutes and a peak corresponding to interferon-beta-1b having a retention time of 33.84 minutes;

FIGS. 2A-B present color images of a non-reducing SDS-PAGE gel slab in which the PEGylation reaction product of recombinant human interferon-beta-1b and 30 kDa methoxy polyethylene glycol N-ethyl-2-iodo-acetamide (as isolated by RP-HPLC) and the starting materials of the PEGylation reaction were run and stained with Coomassie Blue (FIG. 2A) and subsequently with iodine (FIG. 2B), wherein an un-PEGylated sample of interferon-beta-1b was run in lane 1; the isolated peak by RP-HPLC was run in lane 2; molecular weight markers were run in lane 3; and a series of samples of 30 kDa methoxy polyethylene glycol N-ethyl-2-iodo-acetamide (the PEGylation reagent) at various concentrations (0.078 μg, 0.3125 μg, 3.125 μg, 6.25 μg, 12.5 μg and 25 μg) which were run in lanes 4, 5, 6, 7, 8 and 9 respectively;

FIG. 3 presents a MALDI-TOF mass spectrogram of the PEGylation reaction product of recombinant human interferon-beta-1b with 30 kDa methoxy polyethylene glycol N-ethyl-2-iodo-acetamide, using a 2,4-dihydroxybenzoic acid matrix, showing a small peak with an average molecular weight of about 50,000 Da corresponding to the molecular weight of a mono-PEGylated interferon-beta-1b conjugate;

FIG. 4 presents a RP-HPLC chromatogram of the PEGylation reaction product of recombinant human interferon-beta-1b and 30 kDa methoxy polyethylene glycol N-ethyl-(4-bromomethyl)-benzamide, showing a peak having a retention time of 30.27 minutes corresponding to methoxy polyethylene glycol N-ethyl-(4-bromomethyl)-benzamide, a second peak having a retention time of 38 minutes corresponding to the PEGylated protein, and a peak having a retention time of 48.44 minutes corresponding to recombinant human interferon-beta-1b;

FIGS. 5A-B present color images of a non-reducing SDS-PAGE separation gel in which the PEGylation reaction products of recombinant human interferon-beta-1b and 30 kDa methoxy polyethylene glycol N-ethyl-(4-bromomethyl)-benzamide, after collection by RP-HPLC, were run and stained with Coomassie Blue (FIG. 5A) and subsequently with iodine (FIG. 5B), wherein the collected fractions were run in lanes 1, 2 and 3; an un-PEGylated sample of recombinant human interferon-beta-1b was run in lane 4, a 30 kDa methoxy polyethylene glycol N-ethyl-(4-bromomethyl)-benzamide sample was run in lane 5 and molecular weight markers were run in lane 6;

FIG. 6 presents a MALDI-TOF mass spectrogram of the purified PEGylation product of recombinant human interferon-beta-1b with 30 kDa methoxy polyethylene glycol N-ethyl-(4-bromomethyl)-benzamide, using a 2,4-dihydroxybenzoic acid matrix;

FIGS. 7A-B present ESI mass spectrograms of the purified PEGylation product of recombinant human interferon-beta-1b with 30 kDa methoxy polyethylene glycol N-ethyl-(4-bromomethyl)-benzamide (FIG. 7A) and recombinant human interferon-beta-1b (FIG. 7B);

FIGS. 8A-B present RP-HPLC chromatograms showing the peptide maps of purified PEGylation product of recombinant human interferon-beta-1b with 30 kDa methoxy polyethylene glycol N-ethyl-(4-bromomethyl)-benzamide (FIG. 8A), and of recombinant human interferon-beta-1b (FIG. 8B), as obtained by applying peptide digestion using Lys-C;

FIGS. 9A-B present color images of a non-reducing SDS-PAGE separation gel in which the peptides which were formed after peptide Lys-C digestion of the purified PEGylation product of recombinant human interferon-beta-1b with 30 kDa methoxy polyethylene glycol N-ethyl-(4-bromomethyl)-benzamide were run and stained with Coomassie Blue (FIG. 9A) and subsequently with iodine (FIG. 9B), wherein the collected fractions of peptide K3 and the new formed peptide with a retention time of 47.5 minutes were run in lanes 1 and 2 respectively; a 30 kDa methoxy polyethylene glycol N-ethyl-(4-bromomethyl)-benzamide sample was run in lane 3 and molecular weight markers were run in lane 4;

FIG. 10 presents a comparative plot of the concentration of interferon-beta-1b (INF-β1b) in plasma of rats as a function of time, following intravenous administration of 0.4 mg/kg of 30 kDa PEG-Interferon beta-1b (in red) and BETAFERON® (in green), showing the pharmacokinetic profile of the two drugs and the remarkable longer pharmacologic range of the PEGylated interferon according to some embodiments of the present invention;

FIGS. 11A-B present color images of a non-reducing SDS-PAGE gel slab in which the PEGylation reaction products of recombinant human interferon-beta-1a and 30 kDa methoxy polyethylene glycol N-ethyl-2-iodo-acetamide, eluted on a Fractogel COO⁻ column, were run and stained with Coomassie Blue (FIG. 11A) and subsequently with iodine (FIG. 11B), wherein molecular weight markers were run in lane 1; an un-PEGylated sample of interferon-beta-1a was run in lane 2; the eluted fraction was run in lane 3; the unbound fraction washed with sodium acetate buffer solutions was run in lane 4; and the crude reaction mixture which was loaded on the column was run in lane 5; showing the PEGylated protein (marked with an arrow) and an increase of about 50 kDa in the apparent molecular weight of the PEGylated protein as compared to the un-PEGylated protein;

FIGS. 12A-B present color images of a non-reducing SDS-PAGE separation gel in which the PEGylation reaction products of recombinant human interferon-alpha-2a and 30 kDa methoxy polyethylene glycol N-ethyl-2-iodo-acetamide, which were eluted on a Source 15-S column, were run and stained with Coomassie Blue (FIG. 12A) and iodine (FIG. 12B), wherein the fraction eluted with NaC1 solution was run in lane 1; an un-PEGylated sample of interferon-alpha-2a was run in lane 2, and molecular weight markers were run in lane 3; showing the PEGylated protein (marked with an arrow), and an increase of about 50 kDa in the apparent molecular weight of the PEGylated protein as compared to the un-PEGylated protein;

FIG. 13 presents a RP-HPLC chromatogram of the PEGylation reaction products of recombinant human interferon-alpha-2a and 30 kDa methoxy polyethylene glycol N-ethyl-(4-bromomethyl)-benzamide wherein several formed peaks are observed (retention times of 21.77, 23.78, 25.05, 25.57 and 27.42 minutes) and recombinant human interferon-alpha-2a has a retention time of 28.42 minutes;

FIGS. 14A-B present color images of a non-reducing SDS-PAGE gel slab in which the PEGylation reaction products of recombinant human interferon-alpha-2a and 30 kDa methoxy polyethylene glycol N-ethyl-(4-bromomethyl)-benzamide, after collection by RP-HPLC, were run and stained with Coomassie Blue (FIG. 14A) and subsequently with iodine (FIG. 14B), wherein the collected fractions were run in lanes 1, 2 , 3 and 4; an un-PEGylated sample of recombinant human interferon-alpha-2a was run in lane 5, a 30 kDa methoxy polyethylene glycol N-ethyl-(4-bromomethyl)-benzamide sample was run in lane 6 and molecular weight markers were run in lane 7;

FIG. 15 presents a RP-HPLC chromatogram of the PEGylation reaction products of recombinant human erythropoietin and 30 kDa methoxy polyethylene glycol N-ethyl-(4-bromomethyl)-benzamide wherein a new peak is observed with a retention time of 25.62 minutes and recombinant human erythropoietin has a retention time of 28.35 minutes;

FIGS. 16A-B present color images of a non-reducing SDS-PAGE separation gel in which the PEGylation reaction products of recombinant human erythropoietin and 30 kDa methoxy polyethylene glycol N-ethyl-(4-bromomethyl)-benzamide, after collection by RP-HPLC, were run and stained with Coomassie Blue (FIG. 16A) and subsequently with iodine (FIG. 16B), wherein the collected fractions were run in lanes 1 and 2; an un-PEGylated sample of recombinant human erythropoietin was run in lane 3, a 30 kDa methoxy polyethylene glycol N-ethyl-(4-bromomethyl)-benzamide sample was run in lane 4 and molecular weight markers were run in lane 5;

FIGS. 17A-B present RP-HPLC chromatograms, comparing the peptide maps of purified PEGylation product of recombinant human erythropoietin (rh-EPO) with 30 kDa methoxy polyethylene glycol N-ethyl-(4-bromomethyl)-benzamide (FIG. 17A), and of a un-PEGylated hr-EPO (FIG. 17B), as obtained by applying peptide digestion using trypsin;

FIG. 18 presents a RP-HPLC chromatogram of the PEGylation reaction products of recombinant human granulocyte colony stimulating factor and 30 kDa methoxy polyethylene glycol N-ethyl-(4-bromomethyl)-benzamide wherein PEGylated protein peaks are observed with a retention time of 36.9, 48.2 and 49.4 minutes and recombinant human granulocyte colony stimulating factor has a retention time of 53.5 minutes;

FIGS. 19A-B present color images of a non-reducing SDS-PAGE separation gel in which the PEGylation reaction products of recombinant human granulocyte colony stimulating factor and 30 kDa methoxy polyethylene glycol N-ethyl-(4-bromomethyl)-benzamide, after collection by RP-HPLC, were run and stained with Coomassie Blue (FIG. 19A) and subsequently with iodine (FIG. 19B), wherein the collected fractions were run in lanes 1, 2, 3 and 4; an un-PEGylated sample of recombinant human granulocyte colony stimulating factor was run in lane 5, a 30 kDa methoxy polyethylene glycol N-ethyl-(4-bromomethyl)-benzamide sample was run in lane 6 and molecular weight markers were run in lane 7;

FIGS. 20A-B present ESI mass spectrograms of the purified PEGylation product of recombinant human granulocyte colony stimulating factor with 30 kDa methoxy polyethylene glycol N-ethyl-(4-bromomethyl)-benzamide (FIG. 20A) and recombinant human granulocyte colony stimulating factor (FIG. 20B);

FIG. 21 presents a RP-HPLC chromatogram of the PEGylation reaction products of recombinant human growth hormone and 30 kDa methoxy polyethylene glycol N-ethyl-(4-bromomethyl)-benzamide wherein a new formed peak is observed with a retention time of 46.65 minutes and recombinant human growth hormone has a retention time of 52 minutes;

FIGS. 22A-B present color images of a non-reducing SDS-PAGE separation gel in which the PEGylation reaction products of recombinant human growth hormone and 30 kDa methoxy polyethylene glycol N-ethyl-(4-bromomethyl)-benzamide, after collection by RP-HPLC, were run and stained with Coomassie Blue (FIG. 22A) and subsequently with iodine (FIG. 22B), wherein the collected fractions were run in lanes 1, 2, 3; an un-PEGylated sample of recombinant human growth hormone was run in lane 4, a 30 kDa methoxy polyethylene glycol N-ethyl-(4-bromomethyl)-benzamide sample was run in lane 5 and molecular weight markers were run in lane 6; and

FIGS. 23A-B present color images of a non-reducing SDS-PAGE separation gel in which the PEGylation reaction products of recombinant follicular stimulating hormone (rh-FSH) and 30 kDa methoxy polyethylene glycol N-ethyl-(4-bromomethyl)-benzamide without purification were run and stained with Coomassie Blue (FIG. 23A) and subsequently with iodine (FIG. 23B), wherein the reaction mixture after 17 hours was run in lane 1;, the reaction mixture at time zero was run in lane 2;, an un-PEGylated sample of recombinant human follicle stimulating hormone was run in lane 3, a 30 kDa methoxy polyethylene glycol N-ethyl-(4-bromomethyl)-benzamide sample was run in lane 4 and molecular weight markers were run in lane 5.

### DESCRIPTION OF THE PREFERRED EMBODIMENTS

The present invention, in some embodiments thereof, relates to novel conjugates of proteins and of processes of preparing same, and, more specifically, but not exclusively, to conjugates of a protein and one or more polymers which are attached to a side chain of one or more methionine residues within the protein. Embodiments of the present invention also relate to pharmaceutical compositions containing these conjugates and to uses thereof as therapeutic agents for treating various medical conditions. The conjugated proteins of the present embodiments are characterized by improved pharmacokinetic profile, which renders them highly suitable for use in therapeutic applications.

The principles and operation of the present invention may be better understood with reference to the figures and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed hereinabove, therapeutic proteins are prone to evoke an immunogenic response, are relatively water insoluble, and generally suffer from a short and insufficient in vivo half-life. The pharmacokinetics of the particular protein will govern both the efficacy and duration of therapeutic effect of the drug, and therefore it is important to reduce the rate of clearance of the protein so that prolonged action can be achieved.

One of the most effective approaches for accomplishing a better to pharmacokinetic profile for pharmaceutical proteins is effected by attaching a polymer, such as polyethylene glycol (PEG), to the protein. The polymer improves the aqueous solubility of the protein, masks potential epitope and proteolysis sites on the protein, and increases the molecular weight and volume thereof. The attachment of PEG to a protein, known as PEGylation, has been shown to be efficacious in reducing the rate of clearance of the therapeutic protein drug from the physiological system, reducing renal clearance, reducing proteolysis, reducing antigenicity, and increasing water solubility, while retaining a substantial proportion of the biological activity of the therapeutic protein.

As further discussed hereinabove, proteins may be PEGylated in a variety of methods, depending on the type, number and availability of particular functional groups on its surface which are suitable for PEGylation.

While conceiving the present invention, the present inventor have recognized that methionine may serve as a favorable PEGylation site, particularly in cases wherein site-specific or otherwise preferential PEGylation is required due to its relative scarcity and to its unique chemical reactivity.

Table 1 below presents the observed frequency of all naturally occurring amino acids in vertebrates and clearly show the relatively low abundance of methionine. As shown in the structural formula of methionine presented below, this amino acid has a unique thioether group in its side-chain, which is characterized by a unique chemical reactivity with respect to the reactivity of other available functional groups found in other amino acids in proteins.

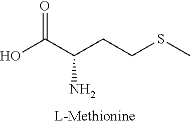

TABLE 1

| | | Observed |
|---|---|---|
| Amino Acids | Codons | Frequency |
| Tryptophan | UGG | 1.3% |
| Methionine | AUG | 1.8% |
| Histidine | CAU, CAC | 2.9% |
| Cysteine | UGU, UGC | 3.3% |
| Tyrosine | UAU, UAC | 3.3% |
| Glutamine | CAA, CAG | 3.7% |
| Isoleucine | AUU, AUA, AUC | 3.8% |
| Phenylalanine | UUU, UUC | 4.0% |
| Arginine | CGU, CGA, CGC, CGG, AGA, | 4.2% |
| Asparagine | AAU, AAC | 4.4% |
| Proline | CCU, CCA, CCC, CCG | 5.0% |
| Glutamic Acid | GAA, GAG | 5.8% |
| Aspartic Acid | GAU, GAC | 5.9% |
| Threonine | ACU, ACA, ACC, ACG | 6.2% |
| Valine | GUU, GUA, GUC, GUG | 6.8% |
| Lysine | AAA, AAG | 7.2% |
| Alanine | GCU, GCA, GCC, GCG | 7.4% |
| Glycine | GGU, GGA, GGC, GGG | 7.4% |
| Leucine | CUU, CUA, CUC, CUG, UUA, | 7.6% |
| Serine | UCU, UCA, UCC, UCG, AGU, | 8.1% |
| Stop Codons | UAA, UAG, UGA | — |

The relatively low abundance and the unique chemistry of its side chain renders methionine a highly suitable target for polymer attachment since it increases the probability of a single and site-directed modification event versus a multiple and/or random attachment points.

PEGylation of proteins via the side-chain of methionine has not been described in the art hitherto.

Another example of a rare amino acid relevant in the context of the present embodiments is selenomethionine. Selenomethionine (Sel-met or Sem) is an amino acid containing selenium instead of the sulfur atom of methionine. The L-isomer of selenomethionine is a naturally occurring amino acid. In vivo, Sel-met is randomly incorporated instead of methionine in lower organisms, and its redox activity stems from its ability to deplete reactive species. As methionine, Sel-met can undergo alkylation (Lang, S. et al., Anal. Biochem., 342, 271-279, 2005; and Lang, S. et al., Anal. Biochem., 359, 253-258, 2006) and may therefore undergo PEGylation according to the present embodiments.

Hence, the present embodiments encompass peptides and proteins which have one or more methionine and/or selenomethionine residues which are either naturally, intentionally, synthetically or genetically incorporated thereto.

The term “methionine”, as used herein, therefore encompasses both methionine and selenomethionine.

Thus, according to one aspect of the present invention there is provided a conjugate which comprises:

(a) a polypeptide having one or more methionine residues in its amino acid sequence, each methionine residue is characterized by a methylsulfanyl-ethyl side-chain; and

(b) one or more polymer moieties being covalently attached to the polypeptide via the sulfur atom of the methylsulfanyl-ethyl side-chain of the methionine residue(s).

The terms “polypeptide” and “protein”, which are used herein interchangeably, refer to a polymeric form of amino acids of any length (e.g., of two or more, or 10 or more and more, or 100 or more amino acids), which can include coded and non-coded amino acids, chemically or biochemically modified or derivatized amino acids, and polypeptides having modified peptide backbones. Polypeptides may be polymers of naturally occurring amino acid residues; non-naturally occurring amino acid residues, such as, for example N-substituted glycine residues, amino acid substitutes, and the like; and both naturally occurring and non-naturally occurring amino acid residues/substitutes. This term does not refer to or exclude post-translational modifications of the polypeptide, for example, glycosylations, acetylations, phosphorylations and the like. The term includes ribosomally or synthetically made polypeptides, fusion proteins, including, but not limited to, fusion proteins with a heterologous amino acid sequence, fusions with heterologous and homologous leader sequences, with or without N-terminal methionine residues; immunologically tagged proteins; and the like. In the context of the present invention, these terms refer to polypeptides and proteins which comprise at least one methionine in their amino acid sequence.

In general, the term “polypeptide”, as used herein, refers to all the polypeptides which can be used in any of the embodiments of the present invention, and encompasses a naturally occurring, a mutated, an altered, a genetically engineered, a synthetic, an extracted and/or a recombinant polypeptide.

As used herein throughout, the term “amino acid” or “amino acids” is understood to include the 20 genetically coded or naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids and other non-naturally occurring amino acids.

Tables 2 and 3 below list the genetically encoded or naturally occurring amino acids (Table 2) and non-limiting examples of non-conventional/modified amino acids (Table 3).

TABLE 2

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 3

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane-carboxylate | Cpro | L-N-methylasparagine | Nmasn |
| aminoisobutyric acid | Aib | L-N-methylaspartic acid | Nmasp |
| aminonorbornyl-carboxylate | Norb | L-N-methylcysteine | Nmcys |
| Cyclohexylalanine | Chexa | L-N-methylglutamine | Nmgin |
| Cyclopentylalanine | Cpen | L-N-methylglutamic acid | Nmglu |
| D-alanine | Dal | L-N-methylhistidine | Nmhis |
| D-arginine | Darg | L-N-methylisolleucine | Nmile |
| D-aspartic acid | Dasp | L-N-methylleucine | Nmleu |
| D-cysteine | Dcys | L-N-methyllysine | Nmlys |
| D-glutamine | Dgln | L-N-methylmethionine | Nmmet |
| D-glutamic acid | Dglu | L-N-methylnorleucine | Nmnle |
| D-histidine | Dhis | L-N-methylnorvaline | Nmnva |
| D-isoleucine | Dile | L-N-methylornithine | Nmorn |
| D-leucine | Dleu | L-N-methylphenylalanine | Nmphe |
| D-lysine | Dlys | L-N-methylproline | Nmpro |
| D-methionine | Dmet | L-N-methylserine | Nmser |
| D/L-ornithine | D/Lorn | L-N-methylthreonine | Nmthr |
| D-phenylalanine | Dphe | L-N-methyltryptophan | Nmtrp |
| D-proline | Dpro | L-N-methyltyrosine | Nmtyr |
| D-serine | Dser | L-N-methylvaline | Nmval |
| D-threonine | Dthr | L-N-methylethylglycine | Nmetg |
| D-tryptophan | Dtrp | L-N-methyl-t-butylglycine | Nmtbug |
| D-tyrosine | Dtyr | L-norleucine | Nle |
| D-valine | Dval | L-norvaline | Nva |
| D-α-methylalanine | Dmala | α-methyl-aminoisobutyrate | Maib |
| D-α-methylarginine | Dmarg | α-methyl-γ-aminobutyrate | Mgabu |
| D-α-methylasparagine | Dmasn | α-methylcyclohexylalanine | Mchexa |
| D-α-methylaspartate | Dmasp | α-methylcyclopentylalanine | Mcpen |
| D-α-methylcysteine | Dmcys | α-methyl-α-napthylalanine | Manap |
| D-α-methylglutamine | Dmgln | α-methylpenicillamine | Mpen |
| D-α-methylhistidine | Dmhis | N-(4-aminobutyl)glycine | Nglu |
| D-α-methylisoleucine | Dmile | N-(2-aminoethyl)glycine | Naeg |
| D-α-methylleucine | Dmleu | N-(3-aminopropyl)glycine | Norn |
| D-α-methyllysine | Dmlys | N-amino-α-methylbutyrate | Nmaabu |
| D-α-methylmethionine | Dmmet | α-napthylalanine | Anap |
| D-α-methylornithine | Dmorn | N-benzylglycine | Nphe |
| D-α-methylphenylalanine | Dmphe | N-(2-carbamylethyl)glycine | Ngln |
| D-α-methylproline | Dmpro | N-(carbamylmethyl)glycine | Nasn |
| D-α-methylserine | Dmser | N-(2-carboxyethyl)glycine | Nglu |
| D-α-methylthreonine | Dmthr | N-(carboxymethyl)glycine | Nasp |
| D-α-methyltryptophan | Dmtrp | N-cyclobutylglycine | Ncbut |
| D-α-methyltyrosine | Dmty | N-cycloheptylglycine | Nchep |
| D-α-methylvaline | Dmval | N-cyclohexylglycine | Nchex |
| D-α-methylalnine | Dnmala | N-cyclodecylglycine | Ncdec |
| D-α-methylarginine | Dnmarg | N-cyclododeclglycine | Ncdod |
| D-α-methylasparagine | Dnmasn | N-cyclooctylglycine | Ncoct |
| D-α-methylasparatate | Dnmasp | N-cyclopropylglycine | Ncpro |
| D-α-methylcysteine | Dnmcys | N-cycloundecylglycine | Ncund |
| D-N-methylleucine | Dnmleu | N-(2,2-diphenylethyl)glycine | Nbhm |
| D-N-methyllysine | Dnmlys | N-(3,3-diphenylpropyl)glycine | Nbhe |
| N-methylcyclohexylalanine | Nmchexa | N-(3-indolylyethyl) glycine | Nhtrp |
| D-N-methylornithine | Dnmorn | N-methyl-γ-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nile | D-N-methylproline | Dnmpro |
| N-(2-methylpropyl)glycine | Nleu | D-N-methylserine | Dnmser |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | Nva |
| γ-aminobutyric acid | Gabu | N-methyla-napthylalanine | Nmanap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| D-N-methylglutamine | Dnmgln | L-α-methylhomo phenylalanine | Mhphe |
| D-N-methylglutamate | Dnmglu | N-(2-methylthioethyl)glycine | Nmet |
| D-N-methylhistidine | Dnmhis | N-(3-guanidinopropyl)glycine | Narg |
| D-N-methylisoleucine | Dnmile | N-(1-hydroxyethyl)glycine | Nthr |
| D-N-methylleucine | Dnmleu | N-(hydroxyethyl)glycine | Nser |
| D-N-methyllysine | Dnmlys | N-(imidazolylethyl)glycine | Nhis |
| N-methylcyclohexylalanine | Nmchexa | N-(3-indolylyethyl)glycine | Nhtrp |
| D-N-methylornithine | Dnmorn | N-methyl-γ-aminobutyrate | Nmgabu |
| N-methylglycine | Nala | D-N-methylmethionine | Dnmmet |
| N-methylaminoisobutyrate | Nmaib | N-methylcyclopentylalanine | Nmcpen |
| N-(1-methylpropyl)glycine | Nile | D-N-methylphenylalanine | Dnmphe |
| N-(2-methylpropyl)glycine | Nleu | D-N-methylproline | Dnmpro |
| D-N-methyltryptophan | Dnmtrp | D-N-methylserine | Dnmser |
| D-N-methyltyrosine | Dnmtyr | D-N-methylthreonine | Dnmthr |
| D-N-methylvaline | Dnmval | N-(1-methylethyl)glycine | Nval |
| γ-aminobutyric acid | Gabu | N-methyla-napthylalanine | Nmanap |
| L-t-butylglycine | Tbug | N-methylpenicillamine | Nmpen |
| L-ethylglycine | Etg | N-(p-hydroxyphenyl)glycine | Nhtyr |
| L-homophenylalanine | Hphe | N-(thiomethyl)glycine | Ncys |
| L-α-methylarginine | Marg | penicillamine | Pen |
| L-α-methylaspartate | Masp | L-α-methylalanine | Mala |
| L-α-methylcysteine | Mcys | L-α-methylasparagine | Masn |
| L-α-methylglutamine | Mgln | L-α-methyl-t-butylglycine | Mtbug |
| L-α-methylhistidine | Mhis | L-methylethylglycine | Metg |
| L-α-methylisoleucine | Mile | L-α-methylglutamate | Mglu |
| L-α-methylleucine | Mleu | L-α-methylhomophenylalanine | Mhphe |
| L-α-methylmethionine | Mmet | N-(2-methylthioethyl)glycine | Nmet |
| L-α-methylnorvaline | Mnva | L-α-methyllysine | Mlys |
| L-α-methylphenylalanine | Mphe | L-α-methylnorleucine | Mnle |
| L-α-methylserine | mser | L-α-methylornithine | Morn |
| L-α-methylvaline | Mtrp | L-α-methylproline | Mpro |
| L-α-methylleucine | Mval Nnbhm | L-α-methylthreonine | Mthr |
| N-(N-(2,2-diphenylethyl)carbamylmethyl-glycine | Nnbhm | L-α-methyltyrosine | Mtyr |
| 1-carboxy-1-(2,2-diphenyl ethylamino)cyclopropane | Nmbc | L-N-methylhomophenylalanine | Nmhphe |
| N-(N-(3,3-diphenylpropyl)carbamylmethyl(1)glycine | Nnbhe | D/L-citrulline | D/Lctr |

As is well accepted in the art, the term “residue”, as used herein, describes a portion, and typically a major portion, of a molecular entity, such as molecule or a part of a molecule such as a group, which has underwent a chemical reaction and is now covalently linked to another molecular entity. For example, the molecular entity can be an amino acid molecule, and the portion of the amino acid which forms a part of a polypeptide chain after the formation of the polypeptide chain, is an amino acid residue. An amino acid residue is therefore that part of an amino acid which is present in a peptide sequence upon reaction of, for example, an alpha-amine group thereof with an alpha-carboxylic group of an adjacent amino acid in the peptide sequence, to form a peptide amide bond and/or of an alpha-carboxylic acid group thereof with an alpha-amine group of an adjacent amino acid in the peptide sequence, to form a peptide amide bond.

For example, in the case of an alkyl halide, the term “residue” describes the alkyl part of the alkyl halide, which is present in a molecule upon being subjected to a nucleophilic substitution reaction, in which the halide serves as a leaving group.

The term “side-chain”, as used herein with reference to amino acids, refers to a chemical group which is attached to the a-carbon atom of an amino acid. The side-chain is unique for each type of amino acid and does not take part in forming the peptide bond which connects the amino acids in a polypeptide. For example, the side chain for glycine is hydrogen, for alanine it is methyl, for valine it is isopropyl and for methionine it is methylsulfanyl-ethyl. For the specific side chains of all amino acids reference is made to A. L. Lehninger's text on Biochemistry (see, chapter 4).

As used herein, the phrase “moiety” describes a part or a major part of a chemical entity or compound, which typically has certain functionality or distinguishing features.

Due to the beneficial features attributed to polypeptides by conjugating thereto a polymeric moiety that alters their pharmacokinetic profile, delineated hereinabove, the polypeptides in the conjugates presented herein can be therapeutic proteins or proteins which otherwise exhibit a beneficial pharmacological and/or diagnostic activity.

In the context of the present embodiments, the phrase “therapeutic protein” describes a protein of any source and origin, synthetic of naturally occurring, which has been identified as having a beneficial therapeutic effect when administered exogenously to a subject.

According to the present embodiments, the polypeptide has at least one methionine (or selenomethionine) residue in its sequence. The methionine (or selenomethionine) can be naturally found in the polypeptide (as a result of natural processes and evolution), and can be entered into the sequence by genetic engineering techniques, for example, having codons for methionine inserted into the corresponding genetic code which is expressed to yield a given polypeptide. According to the present embodiments, the methionine can be entered into the sequence of the polypeptide also by synthetic techniques which are known in the art for preparing relatively short polypeptides.

Hence, according to the some embodiments, the polypeptide may have a methionine (or selenomethionine) inserted into its amino-acid sequence artificially, either as an added amino-acid (insertion mutation), or as a replacement to another amino-acid (replacement mutation). These polypeptide or proteins are modified so as to have a methionine in their amino-acid sequence, and are sometimes referred to as genetically engineered mutant proteins and recombinant proteins. In the context of some embodiments of the present invention, some proteins and polypeptides which do not have a methionine coded for in their native amino-acid sequence are encompassed herein as being modified so as to have at least one methionine residue present therein, and are still referred to in their original name.

According to some embodiments, at least one of the methionine residues present in the polypeptide is accessible to chemical conjugation to a polymer, such as PEGylation, by being positioned at. or close to, the solvent-accessible surface of the fully formed and folded polypeptide. As discussed herein, buried and otherwise less accessible methionine residues side-chains may still undergo chemical conjugation to a polymer under certain conditions and/or by using polymer-conjugation reagents that are selected suitable to this effect.

The phrase “solvent-accessible surface”, as used herein, refers to the surface area of the polypeptide that is accessible to the molecules of the solvent it is dissolved in. The solvent-accessible surface is oftentimes referred to as the Lee-Richards molecular surface [Lee B. and Richards F M., 1971, “The interpretation of protein structures: estimation of static accessibility”, J. Mol. Biol., 55(3), pp. 379-400]. A functional group of an amino-acid residue which is positioned at or near the solvent-accessible surface of a protein is more likely to be available for chemical modifications and polymer conjugation, such as PEGylation.

The protein, according to some embodiments of the present invention, can be any protein which is administered exogenously into a subject, such as a human. Exemplary proteins which are relevant in the context of the present embodiments include, without limitation, therapeutic protein drugs and agents such as interferons, cytokines, hormones, growth factors, blood proteins (blood factors), plasma-derived proteins, urine-derived proteins, antibodies and antigens, enzymes, viral proteins and fusion proteins.

As mentioned above, the polypeptide can consist of a part of a larger protein having either a longer polypeptide chain or more than one polypeptide chain. Hence, according to the present embodiments, the polypeptide can be any part or segment of a protein, e.g. antibody fragments, such as Fab, Fv, and scFv, and therefore any part or segment of a therapeutic protein, provided that it has at least one methionine residue in its sequence.

Depending on their specific therapeutic purpose, most therapeutic proteins for human consumption are of human origins; hence, according to some embodiments, the polypeptides described herein are recombinant human proteins. Other therapeutic proteins, particularly for vaccination and other immunotherapeutic purposes consist or include proteins or segments thereof from pathogenic organisms and other sources.

Representative examples of interferons which can be utilized in the context of the present embodiments include, without limitation, interferon-alpha such as IFNA1, IFNA2, IFNA4, IFNA5, IFNA6, IFNA7, IFNA8, IFNA10, IFNA13, IFNA14, IFNA16, IFNA17 and IFNA21, interferon-beta such as IFNB1 and IFNB3, interferon-lambda such as IFN-λ1, IFN-λ2 and IFN-λ3 also called IL29, IL28A and IL28B respectively, interferon-kappa, interferon-delta, interferon-epsilon, interferon-tau, interferon-omega and interferon-zeta (limitin),

Representative examples of viral proteins and other antigens which can be utilized in the context of the present embodiments include, without limitation, HW proteins, hepatitis B virus envelope protein, porcine transmissible gastroenteritis virus glycoprotein S, SIgA/G, scFv-bryodin 1, Norwalk virus capsid protein, rabies virus glycoprotein, rotavirus enterotoxin and enterotoxigenic, cholera toxin B or A2 subunit, diabetes autoantigen and Escherichia coli enterotoxin.

Representative examples of antibodies which can be utilized in the context of the present embodiments include, without limitation, herpes simplex virus IgG, herpes simplex virus LSC, rituximab, trastuzumab, cetuximab, palivizumab, infliximab and adalimumab.

Representative examples of blood factors which can be utilized in the context of the present embodiments include, without limitation, alpha 2-antiplasmin, antithrombin III, aprotinin, B-deleted domain Factor VIII, cancer procoagulant, Factor I (fibrinogen), Factor II (prothrombin), Factor Ha (activated Factor II), Factor IX (Christmas factor), Factor V (proaccelerin, labile factor), Factor VII (stable factor), Factor VIIa, Factor VIII (antihemophilic factor), Factor X (Stuart-Prower factor), Factor XI (plasma thromboplastin antecedent), Factor XII (Hageman factor), Factor XIII (fibrin-stabilizing factor), fibronectin, heparin cofactor II, high molecular weight kininogen (HMWK), plasminogen, plasminogen activator inhibitor-1 (PAI1), plasminogen activator inhibitor-2 (PAI2), prekallikrein, protein C, protein S, protein Z, protein Z-related protease inhibitor (ZPI), thrombin, Tissue factor (formerly known as Factor III), tissue plasminogen activator (tPA), urokinase and von Willebrand factor.

Representative examples of hormones which can be utilized in the context of the present embodiments include antimullerian hormone (AMH or mullerian inhibiting factor or hormone), adiponectin (Acrp30), adrenocorticotropic hormone (ACTH or corticotropin), angiotensinogen/angiotensin (AGT), antidiuretic hormone (ADH or vasopressin, arginine vasopressin), atrial-natriuretic peptide (ANP or atriopeptin), calcitonin (CT), cholecystokinin (CCK), chorionic gonadotropin (CG(, corticotropin-releasing hormone (CRH), erythropoietin (EPO), follicle-stimulating hormone (FSH), gastrin (GRP), ghrelin, glucagon, glucagons, gonadotropin-releasing hormone (GnRH), growth hormone (GH or hGH), growth hormone-releasing hormone (GHRH), human chorionic gonadotropin (hCG), human placental lactogen (HPL), inhibin, insulin (INS), insulin-like growth factor (IGF or somatomedin), leptin, luteinizing hormone (LH), melanocyte stimulating hormone (MSH or α-MSH), neuropeptide y, oxytocin, parathyroid hormone (PTH), prolactin (PRL), relaxin, secretin, somatostatin (SRIF), thrombopoietin, thyroid-stimulating hormone (TSH), thyrotropin (TSH) and thyrotropin-releasing hormone (TRH).

Representative examples of growth factors and cytokines which can be utilized in the context of the present embodiments include erythropoietin (EPO), thrombopoietin (TPO), granulocyte-macrophage colony stimulating factor (GM-CSF), granulocyte colony stimulating factor (G-CSF), insulin-like growth factor-1 (IGF-1), keratinocyte growth factor (KGF), platelet-derived growth factor (PDGF), bone morphogenetic protein-2 (BMP-2), bone morphogenetic protein-7 (BMP-7), tumor necrosis factor-alpha (TNF-alpha), interferon-alpha-2a (IFN-alpha-2a), interferon-alpha-2b (IFN_alpha-2b), interferon-beta-1a (IFN beta-1a), interferon-beta-1b (IFN beta-1b), interferon-gamma-1b (IFN-gamma-1b), interleukin-1 (IL-1) receptor antagonist, interleukin (IL-2) and interleukin (IL-11).

Representative examples of enzymes which can be utilized in the context of the present embodiments include human-secreted alkaline phosphatase, α1-antitrypsin, heparanase, alglucosidase-alpha, imiglucerase, laronidase, agalsidase-beta, galsulfase, hyaluronidase, alpha-galactosidase, urate oxidase and human dornase-alpha, urokinase, arginase, asparaginase, methioninase, histaminase, adenosine deaminase, catalase, superoxide dismutase and streptokinase.

Representative examples of fusion proteins which can be utilized in the context of the present embodiments include etanercept, alefacept and r-IL-2 diphteria toxin fusion protein.

Exemplary therapeutically active proteins which are suitable for use in the context of the present embodiments include, without limitation, adalimumab, adenosine deaminase, agalsidase-beta, alglucosidase-alpha, alpha-galactosidase, asparaginase, B-deleted domain Factor VIII, bone morphogenetic protein-2 (BMP-2), bone morphogenetic protein-7 (BMP-7), brain-derived neurotrophic factor (BDNF), cetuximab, chorionic gonadotropin (CG), dornase-alpha, erythropoietin (EPO), etanercept, Factor IX, Factor VIIa, Factor VIII, follicle stimulating hormone (FSH), galsulfase, glial cell line derived neurotrophic factor (GDNF), glucagon, granulocyte colony stimulating factor (G-CSF), granulocyte-macrophage colony stimulating factor (GM-CSF), growth hormone (GH), hemoglobin, heparanase, hyaluronidase, imiglucerase, infliximab, insulin-like growth factor-1 (IGF-1), interferon-alpha-2a (IFN alpha-2a), interferon-alpha-2b (IFN alpha-2b), interferon-beta-1a (IFN beta-1a), interferon-beta-1b (IFN beta-1b), interferon-gamma-1b (IFN gamma-1b), interleukin (IL-11), interleukin (IL-2), interleukin-1 (IL-1) receptor antagonist, interleukin-1 receptor antagonist (IL-1ra), keratinocyte growth factor (KGF), laronidase, luteinizing hormone (LH), megakaryocyte growth differentiation factor (MGDF), obesity protein (OB protein or leptin), osteoprotegerin (OPG), parathyroid hormone (PTH or 1-34 segment or PTH 1-34), palivizumab, platelet-derived growth factor (PDGF), Protein C, rituximab, stem cell factor (SCF), streptokinase, thrombin, thrombopoietin (TPO), thyrotropin (TSH), tissue plasminogen activator (tPA), trastuzumab, tumor necrosis factor binding protein (TNFbp), tumor necrosis factor-alpha (TNF-alpha) and urate-oxidase.

In some embodiments, the therapeutically active protein is interferon-alpha-2a (INF-α2a), interferon-beta-1a (INF-β1a), interferon-beta-1b (INF-β1b), erythropoietin (EPO), granulocyte colony-stimulating factor (G-CSF), human growth hormone (h-GH) or human follicle stimulating hormone (h-FSH).

The polymer moiety is attached to the protein in order to endow certain beneficial qualities to the conjugate protein-polymer, and thus allow or improve the pharmacokinetic characteristics of the protein.

The term “polymer” as used herein encompasses one or more of a polymer, a copolymer or a mixture thereof, as well as linear or branched form thereof when applicable.

Hence, the polymer, according to some embodiments of the invention, is:

i) capable of forming a covalent bond and a stable chemical interaction with the polypeptide either directly or via a linking moiety, as defined hereinbelow;

ii) non-toxic, namely, the polymer and/or its metabolites have no harmful effects to a biological system upon administration;

iii) highly soluble in aqueous solutions, so as to endow a less soluble moiety the capacity to dissolve in aqueous solutions;

iv) highly flexible, so as to have the capacity to assume a wide range of conformations so as to have low immunogenicity and low rate of bio-degradation; and

v) of suitable mass and size, in order to endow a protein sufficient mass and protection, as discussed hereinabove.

These characteristics constitute some of the requirements which render a polymer suitable for conjugation with a protein, and in particular a therapeutic protein. As discussed hereinabove, these requirements include endowing the protein with the necessary solubility and bioavailability, protection from proteolysis, masking from the immune system and extended half-life within the biological system it is administered to, and allowing the polymer to have favorable interaction with the protein.

Thus, in some embodiments, the polymer is selected such that when it is conjugated to a protein:

a) the protein-polymer conjugate substantially preserves the characterizing biological activity of the unconjugated protein in physiological conditions;

b) the protein-polymer conjugate is substantially soluble in aqueous and physiological solutions such as saline, even and particularly when the unconjugated protein is less soluble in the same solutions;

c) the characterizing three dimensional structure of the unconjugated protein is substantially preserved when conjugated to the polymer under physiological conditions; and

d) the characterizing half-life of the protein-polymer conjugate under physiological conditions is substantially greater than the half-life of the unconjugated protein under similar conditions.

As discussed hereinabove, one of the rudimentary objectives of PEGylation of polypeptides is to allow a polypeptide to exert its particular biologic activity when used, for example, as an exogenously administered drug. The conjugates described herein are therefore characterized by having a biological activity similar or identical to that of the unconjugated polypeptide, and by retaining the level of that activity at least to some significant extent, while being further advantageously characterized, according to some embodiments, by improved pharmacokinetic features such as solubility, stability and bioavailability, and the like, which are expressed by, for example, extended half-life.

For example, a polymer-conjugated therapeutic protein may exhibit a considerably lower level of bioactivity as compared to the level of bioactivity of the non-conjugated protein, e.g. retaining even less than 10% thereof (due to many possible factors such as hindrance the interaction with a receptor due to the polymer mass and motility), and still be beneficially used for therapeutic and diagnostic purposes. In such cases the contribution in the extension of a protein's half-life in a biologic system (e.g., the blood) may contribute to the efficacy and usefulness of a conjugated protein as a drug even more than preserving the original level of its bioactivity.

Exemplary polymers which are suitable for conjugation with a polypeptide according to the present embodiments include, without limitation, a polyalkylene glycol, a polyethylene glycol (PEG), a poly(lactic acid) (PLA), a polyester, a polyglycolide (PGA), a polycaprolactone (PCL), a polyamide, a polymethacrylamide, a polyvinyl alcohol, a polycarboxylate, a polyvinyl pyrrolidinone, a dextran, a cellulose, a chitosan, a hydroxyethyl starch (HES), polyamino acids such as polyglutamic acid and polyglycine and any copolymer thereof.

The molecular weight of a polymer determines many of its physical and biochemical properties. In the context of physical properties, the molecular weight of a polymer will determine properties such as, for example, the temperatures for transitions from liquids to waxes to rubbers to solids, and mechanical properties such as stiffness, strength, viscoelasticity, toughness and viscosity. In the context of biochemical properties, the molecular weight of a polymer will determine properties such as, for example, the hydration of a polymer and thus its aqueous solubility, cross-section size which influences the rate of clearance from a biological system, immunogenicity and rate of bio-degradation.

Due to the process of preparing polymers, it is impractical to attempt to achieve a uniform sample of a polymer in which all the molecules have the same length (which corresponds to the molecular weight thereof). Therefore, polymers are characterized by a molecular weight distribution and an average molecular weight.

In the context of the present embodiments, the polymer is selected so as to have an optimal average molecular weight which is suitable for the protein to be conjugated to, and for the particular use of the conjugate.

In some embodiments, the polymer moiety has an average molecular weight which ranges from about 1 kDa to about 100 kDa.

Polyalkylene glycol is a general name which refers to a family of polyether polymers which share the following general formula: HO—[(CH₂)n-O-]m-CH₂OH, wherein n represents the number of methylene groups present in each monomer unit, and m represents the number of repeating monomer units, and therefore represents the size of the polymer. For example, when n=2, the polymer is referred to as polyethylene glycol, and when n=3, the polymer is referred to as polypropylene glycol.

As discussed hereinabove, polyethylene glycol (PEG) is a highly suitable polymer for conjugation with proteins and, hence, according to some embodiments, the polymer is PEG. In some embodiments, the PEG moiety has an average molecular weight that ranges from 4 kDa to 40 kDa. In some embodiments, the PEG moiety is a 30 kDa methoxy polyethylene glycol moiety. A methoxy polyethylene glycol moiety is a polyalkylene glycol residue that terminates with a methoxy (—OCH₃) group (instead of hydroxy).

Derivatives of polyalkylene glycols are therefore also contemplated. These include, for example, polyalkylene glycols in which one of more of the methylene groups in the repeating monomer units is substituted by, for example, an alkyl, a alkenyl, a cycloalkyl, an alkoxy, a thioalkoxy, a halide, and more.

According to some embodiments, the polypeptide and the polymer moiety are covalently attached to each other at the sulfur atom of a methionine side-chain via a linking moiety.

As used herein, the phrase “linking moiety” describes a chemical moiety or a group, as defined herein, which links the polymeric moiety and the polypeptides. The linking moiety can thus be, for example, formed upon reacting a reactive moiety within the polymer with the thioether group at the side chain of a methionine.

Thus, according to some embodiments of the present invention, the linking moiety includes at least one residue of a reactive moiety, whereby the reactive moiety is selected capable of reacting with a sulfur atom of a methionine side-chain.

As discussed in further details hereinbelow, in some embodiments of the invention, the conjugation reaction of the polymer moiety to the sulfur atom of the methionine side-chain is effected via an alkylation, or a methylation, reaction, and according to some embodiments, it is a nucleophilic substitution of a leaving group on the reactive moiety of an alkylating agent by the sulfur atom. Hence, the linking moiety, according to some embodiments, comprises a residue, as defined herein, of a reactive moiety, whereby the residue is formed upon an interaction of the reactive moiety with the thioether group of the methionine residue

The phrase “reactive moiety”, as used herein, describes a chemical group that is capable of undergoing a chemical reaction that typically leads to a bond formation. The bond, according to some embodiments, is a covalent bond. Chemical reactions that lead to a bond formation include, for example, nucleophilic and electrophilic substitutions, nucleophilic and electrophilic addition reactions, alkylations, addition-elimination reactions, cycloaddition reactions, rearrangement reactions and any other known organic reactions that involve a reactive group.

For example, according to some embodiments, the reactive moiety which is capable of reacting with the sulfur atom of the side-chain of a methionine, comprises a leaving group, as defined hereinbelow, and can be selected capable of alkylating the sulfur atom by means of a nucleophilic substitution.

As discussed hereinabove, some cases wherein the methionine is not at, or close to, the solvent-accessible surface of the polypeptide, the conjugation of the polymer thereto can be effected by means of an “elongation arm”, or a spacer, which can penetrate through the amino-acid residues on the surface of the polypeptide and reach a buried or less accessible methionine, owing to a thin and flexible structure thereof. Thus, according to some embodiments of the present invention, the linking moiety further comprises a spacer.

For the purposes of this invention, the term “spacer” or “spacer moiety” is intended to encompass any chemical entity that covalently connects between two or more compounds, moieties or residues thereof, such as a polypeptide and a polymer and/or a linking moiety. In the context of the present embodiments, the spacer covalently connects between two moieties or residues thereof, one is the first reactive moiety which is attached to the sulfur atom in the side-chain of a methionine residue in the polypeptide, and the other is the second reactive moiety which is attached to the polymer moiety. Further in the context of the present embodiments, such spacer moieties can be designed to facilitate, modulate, regulate or otherwise influence the attachment of the polymer to a methionine side-chain on a polypeptide, particularly in cases where the methionine side-chain is substantially buried within the polypeptide chain or otherwise less accessible for chemical modifications.

Spacer groups, as described herein, include, but are not limited to, a linear or branched, saturated or unsaturated, substituted or unsubstituted alkylene —(CH₂)ₙ— chain having 1-30 carbon atoms (n is an integer ranging from 1 to 30), and a linear or branched, saturated or unsaturated, substituted or unsubstituted alkylene chain having 1-30 carbon atoms interrupted by at least one heteroatom, whereby the at least one heteroatom is selected from the group consisting of oxygen, sulfur, nitrogen, phosphor and/or silicon.

In some embodiments, the spacer moiety is a linear, unsubstituted alkylene chain wherein n is 1, as in methane-di-yl; n is 2, as in ethane-1-yl-2-yl; n is 3, as in propane-1-yl-3-yl; and n is 4, as in butane 1-yl-4-yl.

Alternatively, the spacer can comprise one or more aryl or heteroaryl groups. An example of a spacer moiety is a 1,4-benzene-diyl moiety. If more than one aryls or heteroaryls are present, they can be linked to one another such that a chain of aromatic rings forms the spacer moiety. An example of a spacer moiety made of two benzene rings is a 1,10-biphenyl-diyl moiety.

As discussed hereinabove, the conjugation of a polymer moiety to a polypeptide can be conducted under conditions that enable preferential conjugation at a sulfur atom of a methionine residue side-chain. Thus, according an additional aspect of the present invention there is provided a process of preparing the conjugate presented herein. The process is effected by providing a polymer which has, or is modified so as to have, at least one reactive moiety attached thereto, and reacting this polymer with the polypeptide under acidic conditions, to thereby obtain the conjugate. The acidic conditions of the conjugation reaction are set to range from pH of 2 to pH of 5, or from pH of 3 to pH of 5, such that the reactive moiety, which is selected capable of reacting with a sulfur atom of a methionine side-chain, forms a covalent bond with the sulfur.

It should be noted that while reacting the polymer with the polypeptide can be performed under various (e.g., acidic, basic or neutral) conditions, it can be conducted at a pH range of from pH of 2 to pH of 5, so as to render the reaction specific to methionine side-chains, while avoiding reactions of the polymer with other functional groups found in proteins, and particularly while avoiding conjugation of the polymer to functional groups of the N-terminal amino acid, and of histidine, lysine and cysteine residues, as discussed hereinabove.

Representative examples of a reactive moiety, as defined hereinabove, and is also referred to hereinbelow as the first reactive group, include, but are not limited to, amine, carboxyl, amide, acetamide, 2-halo-acetamide, (4-halomethyl)-benzamide, benzyl-halide, hydrazine, hydrazide, acetohydrazide, alkyl, haloalkyl, alkyl sulfonylhalide, alkyl tosylate, alkyl triflate, allyl, haloallyl, allyl sulfonylhalide, allyl tosylate, allyl triflate, aryl, haloaryl heteroaryl, 4-(halomethyl)benzyl, benzyl, halobenzyl, and any combination thereof.

In some embodiments, the reactive moiety is amide, 2-halo-acetamide, (4-halomethyl)-benzamide, benzyl-halide, haloalkyl, hydrazine, hydrazide or acetohydrazide.

As used herein, the term “amine” describes a —NR′R″ group where each of R′ and R″ is independently hydrogen, alkyl, cycloalkyl, heteroalicyclic, aryl or heteroaryl, as these terms are defined herein.

As used herein, the term “alkyl” describes an aliphatic hydrocarbon including straight chain and branched chain groups. According to some embodiments, the alkyl group has 1 to 20 carbon atoms, or 1-10 carbon atoms. Whenever a numerical range; e.g., “1-10”, is stated herein, it implies that the group, in this case the alkyl group, may contain 1 carbon atom, 2 carbon atoms, 3 carbon atoms, etc., up to and including 10 carbon atoms. The alkyl can be substituted or unsubstituted. When substituted, the substituent can be, for example, an alkyl, an alkenyl, an alkynyl, a cycloalkyl, an aryl, a heteroaryl, a halide, a hydroxy, an alkoxy and a hydroxyalkyl as these terms are defined hereinbelow. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “carboxyl”, as used herein, refers to a —C(═O)—O—R′, where R′ is as defined herein.

The term “amide” describes a —NR′—C(═O)— group, a —NR′—C(═O)—R″ group or a —C(═O)—NR′R″ group, wherein R′ is as defined herein and R″ is as defined for R′. For example, an acetamide refers to a —NR′—C(═O)—CR″— group.

As used herein, the term “hydrazine” describes a —NR′—NR″R′″ group, wherein R′ is as defined herein and R″ and R′″ are as defined for R′.

The term “hydrazide”, as used herein, refers to a —C(═O)—NR′—NR″R′″ group wherein R′, R″ and R′″ are each independently hydrogen, alkyl, cycloalkyl or aryl, as these terms are defined herein. For example, acetohydrazide refers to a —CH₂—C(═O)—NR′—NR″R″ group.

As used herein, the terms “halo” and “halide”, which are referred to herein interchangeably, describe an atom of a halogen, that is fluorine, chlorine, bromine or iodine, also referred to herein as fluoride, chloride, bromide and iodide.

The term “haloalkyl” describes an alkyl group as defined above, further substituted by one or more halide(s).

The term “alkoxy” refers to a —OR′ group, were R′ is as defined herein.

The term “thioalkoxy” refers to a —SR′ group, were R′ is as defined herein.

The term “sulfonylhalide” refers to a —S(═O)₂—X, wherein X is a halide. For example, an alkyl sulfonylhalide refers to a —R—S(═O)₂—X wherein X is a halide and R is an alkyl.

The term “allyl” refers to a —C—C═C group.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted by one or more substituents, as described hereinabove.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazole, pyridine, pyrrole, oxazole, indole, purine and the like.

In some embodiments of the present invention, the conjugation reaction is effected via an alkylating reaction, while utilizing the unique chemical activity of the thioether side chain of methionine. Without being bound by any particular theory, it is assumed that alkylation is effected via a nucleophilic substitution reaction, in which the nucleophilic sulfur atom in the thioether side chain of methionine participates. Hence, according to some embodiments, the reactive moiety comprises an alkyl group and a leaving group.

Such a nucleophilic alkylation reaction typically produces a sulfonium ion, namely, a positively charged sulfur atom carrying three alkyl groups as substituents (S⁺R₃). Thus, the product of such a sulfur-alkylation reaction is typically a sulfonium salt consisting of a sulfonium ion (cation) and a counter ion (a counter-anion). The counter anion can be the leaving group itself, as it is obtained upon its release during the nucleophilic reaction. Otherwise, the leaving group can be replaced by any other chemically compatible moiety. According to some embodiments, the counter ion in the obtained sulfonium salt forms a pharmaceutically acceptable sulfonium salt.

The phrase “pharmaceutically acceptable salt”, as used herein, refers to a salt form of the conjugates presented herein, having a positively charged sulfonium ion and a pharmaceutically acceptable counter ion, which possesses properties such as absorption, distribution, metabolism, excretion and toxicity that render it suitable for use in as a pharmaceutical agent.

Exemplary pharmaceutically acceptable counter-anions include, without limitation, acetate, ascorbate, aspartate, benzenesulfonate, benzoate, besylate, camphorsulfonate, citrate, decanoate, esylate, fumarate, glutamate, glycollate, halide (fluoride, chloride, bromide and iodide), hexanoate, isethionate, lactate, malate, maleate, methanesulfonate (mesylate), naphthalenesulfonate (napsylate), naphthylsulfonate, nitrate, octanoate, oleate, oxalate, pamoate, phosphate (orthophosphate), polystyrene sulfonate, propionate, salicylate, stearate, succinate, sulfate, tartrate and toluenesulfonate (tosylate). In some embodiments, the pharmaceutically acceptable counter-anion is a halide, namely fluoride, chloride, bromide or iodide.

As used herein, the phrase “leaving group” describes a labile atom, group or chemical moiety that readily undergoes detachment from an organic molecule during a chemical reaction, while the detachment is facilitated by the relative stability of the leaving atom, group or moiety thereupon. Typically, any group that is the conjugate base of a strong acid can act as a leaving group. Representative examples of suitable leaving groups according to the present embodiments therefore include, without limitation, halide, acetate, tosylate, triflate, sulfonate, azide, hydroxy, thiohydroxy, alkoxy, cyanate, thiocyanate, nitro and cyano.

The term “acetate” refers to acetic acid anion.

The term “tosylate” refers to toluene-4-sulfonic acid anion.

The term “triflate” refers to trifluoro-methanesulfonic acid anion.

The term “azide” refers to an N₃⁻.

The terms “hydroxy” and “thiohydroxy” refer to the OH⁻ and SH⁻ anions respectively.

The term “cyanate” and “thiocyanate” refer to [O═C═N]⁻ and [S═C═N]⁻ anions respectively.

The term “nitro” refers to NO₂⁻.

The term “cyano” refers to [C≡N]⁻.

In some embodiments, the leaving group is halide.

In order to further facilitate the alkylation reaction, according to some embodiments, the reactive moiety further comprises an electron withdrawing activating group. Therefore, according to some embodiments of the present invention, the reactive moiety can comprise, for example, a short alkyl (e.g., methylene) substituted by a halide as a leaving group and an activating electron withdrawing group, such as an amide, that renders the carbon atom more susceptible to a nucleophilic substitution by the sulfur atom of a methionine side-chain. In another example, the reactive moiety can comprise a short alkyl (e.g., methylene) which is substituted with a tosylate as a highly reactive leaving group, and an allyl as an activating electron withdrawing group. Alternatively, the reactive moiety can comprise a short alkyl (e.g., methylene) which is substituted with a triflate as a highly reactive leaving group, and a benzyl as an activating electron withdrawing group.

The reactive moiety can thus be a combination of several moieties, and according to some embodiments, of a short alkyl, substituted by a reactive leaving group, and further linked to an activating electron withdrawing group. The reactive moiety can thus be a combination of, for example, haloalkyl, haloalkyl-amide, (4-halomethyl)-benzamide, halo-benzyl, halosulfonyllalykl, halotosylalkyl and the like with amide, allyl, aryl and the like.

In some embodiments the reactive moiety is a haloalkyl, a haloalkyl-amide, (4-halomethyl)-benzamide and/or halo-benzyl.

In general, the conjugation reaction between the polypeptide and the polymer moiety can use commercially available PEG reagents, as demonstrated in the Examples section that follows. For example, the PEGylation of recombinant human interferon-beta-1b (rh-IFN-β1b) can be effected with a commercially available 30 kDa methoxy polyethylene glycol N-ethyl-2-iodo-acetamide, as demonstrated in Example 2 hereinbelow, and illustrated in Scheme 1 below. The same PEG reagent was used in Examples 8 and 9 in the PEGylation of rh-IFN-β1a and rh-IFN-α2a respectively.

The PEGylation reaction can further be achieved with other PEGylation reagents, such as, for example, 4-halobenzyl (also referred to herein as benzyl halide), and haloallyl.

Thus, in other embodiments, the PEG reagent, 30 kDa methoxy polyethylene glycol N-ethyl-(4-bromomethyl)-benzamide, is prepared (see, Example 3 hereinbelow) and used in the PEGylation of rh-IFN-β1b and rh-IFN-α2a, EPO, rh-G-CSF; rh-GH and rh-FSH (see, Examples 4, 10, 11, 13, 14 and 15 respectively), as illustrated in Scheme 2 below.

The general concept which is shared by PEGylation reagents, according to some embodiments of the invention, is the attachment of the PEG moiety to the polypeptide via a reactive moiety. The reactive moiety can be attached in one or more steps, namely be first attached to one of the members of the conjugate, and then to the other, or to both of the members of the conjugate in one reaction.

Hence, the exemplary reactive moieties in the PEGylation reagents methoxy polyethylene glycol N-ethyl-2-iodo-acetamide and methoxy polyethylene glycol N-ethyl-(4-bromomethyl)-benzamide, can be regarded as having two functional groups, wherein one functional group is already attached to the PEG, represented by the N-ethyl acetamide moiety in the case of methoxy polyethylene glycol N-ethyl-2-iodo-acetamide and in the case of methoxy polyethylene glycol N-ethyl-(4-bromomethyl)-benzamide, and another functional group used to react with the sulfur atom of the methionine residue in the polypeptide and forms a methionine sulfonium bond, such as the 2-iodo-acetamide or the (bromomethyl)benzene, respectively.

An exemplary conjugate according to the present embodiments is a conjugate of interferon-beta-1b and 30 kDa methoxy polyethylene glycol N-ethyl-acetamide, being attached therebetween via a sulfur atom of a methionine residue of the interferon-beta-1b (see, Example 2), having a formula:

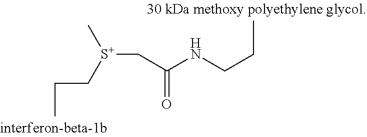

Similarly, another exemplary conjugate comprises interferon-beta-1b and 30 kDa methoxy polyethylene glycol N-ethyl-(4-bromomethyl)-benzamide, being attached therebetween via a sulfur atom of a methionine residue of the interferon-beta-1b (see, Example 4), and having a formula:

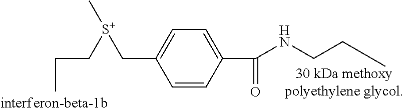

Another exemplary conjugate comprises interferon-beta-1a and 30 kDa methoxy polyethylene glycol N-ethyl-acetamide, being attached therebetween via a sulfur atom of a methionine residue of the interferon-beta-1a (see, Example 8), and having a formula:

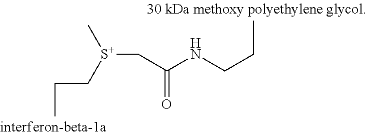

Another exemplary conjugate comprises interferon-alpha-2a and 30 kDa methoxy polyethylene glycol N-ethyl-acetamide, being attached therebetween via a sulfur atom of a methionine residue of the interferon-alpha-2a (see, Example 9), and having a formula:

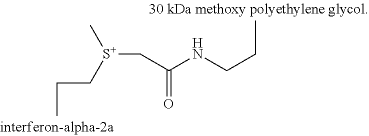

Another exemplary conjugate comprises interferon-alpha-2a and 30 kDa methoxy polyethylene glycol N-ethyl-(4-bromomethyl)-benzamide, being attached therebetween via a sulfur atom of a methionine residue of the interferon-alpha-2a (see, Example 10), and having a formula:

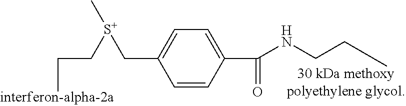

Another exemplary conjugate comprises erythropoietin and 30 kDa methoxy polyethylene glycol N-ethyl-(4-bromomethyl)-benzamide, being attached therebetween via a sulfur atom of a methionine residue of the erythropoietin (EPO, see, Example 11), and having a formula:

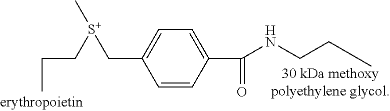

The PEGylated EPO conjugate demonstrates a unique feature of site-specific PEGylation, stemming from the fact that EPO contains a single (site-specific) and accessible (reactive) methionine residue in its sequence.

In the general case, the peptide may have more than one methionine with varying reactivities, allowing situations where the peptide is PEGylated with more than one PEG moiety in a non-homogeneous distribution, affording a population of more than one conjugate species which are distributed into groups according to the reactivity of the methionine residues in the peptide.

Another exemplary conjugate comprises granulocyte colony-stimulating factor (G-CSF) and 30 kDa methoxy polyethylene glycol N-ethyl-(4-bromomethyl)-benzamide, being attached therebetween via a sulfur atom of a methionine residue of the G-CSF (see, Example 13), and having a formula:

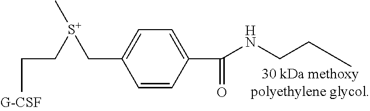

Another exemplary conjugate comprises human growth hormone (h-GH) and 30 kDa methoxy polyethylene glycol N-ethyl-(4-bromomethyl)-benzamide, being attached therebetween via a sulfur atom of a methionine residue of the h-GH (see, Example 14), and having a formula:

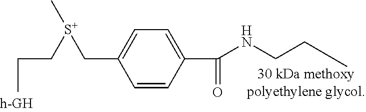

Another exemplary conjugate comprises human follicle stimulating hormone (h-FSH) and 30 kDa methoxy polyethylene glycol N-ethyl-(4-bromomethyl)-benzamide, being attached therebetween via a sulfur atom of a methionine residue of the h-FSH (see, Example 15), and having a formula:

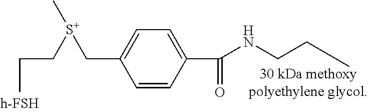

As demonstrated in the Examples section that follows, a novel PEGylation reagent has been designed, prepared and successfully practiced in methionine PEGylation. This reagent is based on a benzyl halide moiety, as a moiety that is highly susceptible to nucleophilic substitution reactions, linked to a polyethylene glycol moiety.

Hence, according to another aspect of embodiments of the invention, there is provided a compound, or a reactive polyalkylene glycol compound, which includes a polyalkylene glycol moiety and a benzyl halide moiety, being covalently linked therebetween via a linking moiety.

According to some embodiments, the polyalkylene glycol moiety is a polyethylene glycol (PEG), and more specifically, the reactive polyalkylene glycol compound comprises a polyethylene glycol which has an average molecular weight that ranges from 4 kDa to 40 kDa. According to some embodiments, the reactive polyalkylene glycol compound comprises a polyethylene glycol which has an average molecular weight that ranges from 20 kDa to 40 kDa. In an exemplary embodiment, the PEG has a molecular weight of about 30 kDa.

The linking moiety which covalently connects between the polyalkylene glycol moiety and the benzyl halide moiety can be, for example, amine, alkyl, aryl, heteroaryl, carboxyl, amide, hydrazine, hydrazide and any combination thereof.

The linking moiety can further comprise a spacer moiety, as defined hereinabove, to facilitate the conjugation of the polyalkylene glycol moiety to the side-chains of less accessible methionine residues of a polypeptide. The spacer therefore links between the benzyl halide moiety and the polyalkylene glycol moiety.

According to some embodiments, the spacer is a linear, saturated, unsubstituted alkylene chain having 1-10 carbon atoms, and in some embodiments, the alkylene chain has 2-4 carbon atoms. In an exemplary embodiment, the spacer is an ethylene (n=2).

It should be noted in this regard that similar PEGylation reagents, having other leaving groups instead of halides, are also contemplated. Hence, the compound described herein can include, for example, a benzyl sulfonyl halide or a benzyl triflate instead of benzyl halide.

As demonstrated in the Examples section that follows, 30 kDa methoxy polyethylene glycol N-ethyl-(4-bromomethyl)-benzamide, a novel PEGylation reagent was successfully prepared (see, Example 3) and utilized (see, Examples 4, 10, 11, 13, 14 and 15 respectively). Thus, an efficient PEGylation of interferon-beta-1b, interferon-alpha-2a, EPO, GCSF, h-GH (human growth hormone) and h-FSH was afforded by using a 4-bromomethyl-N-(PEG)-benzamide reagent at acidic conditions.

As discussed herein, in cases where the methionine is not at, or close to, the solvent-accessible surface of the polypeptide, or in cases where the polymer moiety is bulky such that the conjugation process thereof to the polypeptide becomes inextricable, the conjugation process can be effected via an intermediate compound wherein a modifying moiety is attached to the sulfur atom of a methionine side-chain under favorable conditions, and thereafter the polymer moiety is attached to the modifying moiety. Thus, according to another aspect of embodiments of the invention, there is provided a compound, or a modified polypeptide compound, which includes:

(a) a polypeptide having at least one methionine residue; and

(b) at least one modifying moiety which comprises a residue of a first reactive moiety and a functional moiety, as these are defined hereinabove.

In this modified polypeptide compound, the modifying moiety is covalently attached to the sulfur atom of the methionine side-chain via a residue of a first reactive moiety, which is selected capable of reacting with the sulfur atom.

According to some embodiments of this aspect, the polypeptide is a therapeutically active polypeptide such as, for example, agalsidase-beta, alglucosidase-alpha, alpha-galactosidase, B-deleted domain Factor VIII, bone morphogenetic protein-2 (BMP-2), bone morphogenetic protein-7 (BMP-7), brain-derived neurotrophic factor (BDNF), cetuximab, chorionic gonadotropin (CG), dornase-alpha, erythropoietin (EPO), etanercept, Factor IX, Factor VIIa, Factor VIII, follicle stimulating hormone (FSH), galsulfase, glial cell line derived neurotrophic factor (GDNF), glucagon, granulocyte colony stimulating factor (G-CSF), granulocyte-macrophage colony stimulating factor (GM-CSF), growth hormone (GH), hemoglobin, heparanase, hyaluronidase, imiglucerase, insulin-like growth factor-1 (IGF-1), interferon-alpha-2a (IFN alpha-2a), interferon-alpha-2b (IFN alpha-2b), interferon-beta-1a (IFN beta-1a), interferon-beta-1b (IFN beta-1b), interferon-gamma-1b (IFN gamma-1b), interleukin (IL-11), interleukin (IL-2), interleukin-1 (IL-1) receptor antagonist, interleukin-1 receptor antagonist (IL-1ra), keratinocyte growth factor (KGF), laronidase, luteinizing hormone (LH), megakaryocyte growth differentiation factor (MGDF), obesity protein (OB protein or leptin), osteoprotegerin (OPG), parathyroid hormone (PTH or 1-34 segment or PTH 1-34), platelet-derived growth factor (PDGF), Protein C, rituximab, stem cell factor (SCF), thrombin, thrombopoietin (TPO), thyrotropin (TSH), tissue plasminogen activator (tPA), trastuzumab, tumor necrosis factor binding protein (TNFbp), tumor necrosis factor-alpha (TNF-alpha) and urate-oxidase.

The functional moiety, according to some embodiments, is selected so as to allow conjugation of other moieties to the modified polypeptide, via the modifying moiety. According to some embodiments, the functional moiety is a second reactive moiety. The second reactive moiety can be, for example, amine, carboxyl, amide, hydrazine, hydrazide, thiol, hydroxyl and hydroxylamine, and any combination thereof, including any moiety that enables covalent attachment of a desired moiety.

The term “hydroxyl”, as used herein, refers to an —OH group.

The term “thiohydroxyl” or “thiol”, as used herein, refers to an —SH group.

The term “hydroxylamine”, as used herein, refers to a —NR′—OH group, wherein R′ is as defined herein.

According to some embodiments, the modifying moiety includes a hydrazine or a hydrazide as a second reactive moiety. These second reactive moieties are useful due to their reactivity towards amines under mild conditions, and the widely available chemistry by which an amine can be introduced to a polymer moiety.

As discussed hereinabove, such a modified polypeptide compound can be used in cases where the methionine side-chain is less accessible and hence, the modifying moiety, according to some embodiments, further comprises a spacer which connects the residue of the first reactive moiety and the second reactive moiety. By means of this spacer, a polypeptide which has a buried methionine side-chain (meaning not at or close to the solvent-accessible surface of the polypeptide) can be conjugated to a polymer moiety, and even to a bulky polymer moiety.

Apart from being useful for conjugation with polymer moieties, a modified polypeptide, having a modifying moiety attached to the sulfur atom thereof, may be utilized for covalently attaching to the polypeptide, via its methionine side chain, an additional moiety such as, for example, a labeling moiety. Such modified and labeled polypeptide can be used for various medical, analytical, imaging and diagnostic purposes.

As used herein, the phrase “labeling moiety” refers to a detectable moiety or a probe and includes, for example, fluorescent moieties, phosphorescent moieties, chromophores, phosphorescent moieties, heavy metal clusters, magnetic moieties and radioactive labeling moieties, as well as any other known detectable moieties and any combination thereof.

As used herein, the term “chromophore” refers to a chemical moiety that, when attached to another molecule, renders the latter colored and thus visible when various spectrophotometric measurements are applied.

The phrase “fluorescent moiety” refers to a moiety that emits light at a specific wavelength during exposure to radiation from an external source.

The phrase “phosphorescent moiety” describes a moiety emitting light without appreciable heat or external excitation as by slow oxidation of phosphorous.

A heavy metal cluster can be for example a cluster of gold atoms used, for example, for labeling in electron microscopy techniques.

For example, by attempting to label a given therapeutically active polypeptide with a detectible moiety, according to embodiments presented herein, one can verify whether a methionine side-chain is accessible for a beneficial conjugation with a polymer moiety. As presented in the Examples section that follows, a therapeutically active polypeptide labeled with Lucifer Yellow (a well-known polar tracer for neurons) as labeling moiety can be used to verify whether a PEG moiety could be attached to a given polypeptide (see, Example 16).

The modified polypeptide compound presented herein can be prepared using conditions similar to those used for the preparation of the polymer-polypeptide conjugate presented hereinabove. Thus, according to another aspect of the present invention there is provided a process of preparing the modified polypeptide compound described herein, which is effected by reacting a polypeptide with a modifying moiety which has a first reactive moiety and a second reactive moiety under acidic conditions ranging from pH 2 to pH 5.

The first reactive moiety and the second reactive moiety are selected such that a covalent bond is selectively formed between the first reactive moiety and the sulfur atom, thereby obtaining the modified polypeptide compound. The particular selection of the first reactive moiety and the second reactive moiety should be considered since the desired effect is a reaction between the first reactive group and the protein, leaving the second reactive moiety unaffected and possibly free for another reaction at different condition with, for example, a polymer moiety or a labeling moiety. This discriminating reactivity with respect to the first reactive moiety versus the second reactive moiety can be achieved by selecting two moieties which are reactive under different conditions, or by protecting the second reactive group with an acid-proof protecting group such that it will not be removed during the reaction in which the reactive moiety forms a covalent bond with the sulfur.

An example of such selective reactivity can be achieved with an alkyl halide for one reactive group and an amide for the other reactive group. Similarly, a hydrazine and an acyl-halide can be used having discriminating reactivity.

Hence, according to another aspect of embodiments of the invention there is provided a process of preparing a conjugate which comprises:

a) a polypeptide having at least one methionine residue; and

b) at least one polymer moiety attached to a sulfur atom of a methylsulfanyl-ethyl side-chain of said at least one methionine residue, which is effected by reacting a polypeptide with a modifying moiety as presented hereinabove, under acidic conditions ranging from pH 2 to pH 5, and thereafter reacting this modified polypeptide having at least one modifying moiety attached thereto with a polymer having a third reactive moiety which is selected capable of reacting with the second reactive moiety of the modifying moiety, thereby obtaining the conjugate.

In other words, this two-step process is based on a first step wherein a hetero-bifunctional moiety (a moiety having at least two different reactive moieties) is attached to a polypeptide, thereby affording a modified polypeptide, and a second step wherein a polymer is attached to that moiety of the modified polypeptide, thereby affording a polypeptide-polymer conjugate.

According to some embodiments of the present invention, the third reactive moiety, which forms a part of the polymer moiety, is selected capable of interacting with the second reactive moiety, which forms a part of the modifying moiety, and covalently attach the polymer moiety thereto. Exemplary such (third) reactive groups include, but are not limited to, amine, carboxyl, amide, hydrazine, hydrazide, thiol, hydroxyl and hydroxylamine, and any combination thereof.

As discussed hereinabove, the conjugates presented herein can be used in a variety of medical, diagnostic and other pharmaceutical and therapeutic purposes. In any of the purposes mentioned herein, the conjugates of the present embodiments can be utilized either per se or, alternatively, as a part of a pharmaceutical composition that further comprises a pharmaceutically acceptable carrier.

Thus, according to another aspect of the present invention, there is provided a pharmaceutical composition which comprises a pharmaceutically acceptable carrier and, as an active ingredient, a conjugate as presented herein, which includes:
(a) a polypeptide, as defined and exemplified hereinabove, which has at least one methionine residue in its amino-acid sequence; and
(b) at least one polymer moiety being covalently attached to the sulfur atom a methionine side-chains of at least one the methionine residues.

Accordingly, there is provided a use of a conjugate as presented herein in the manufacture of a medicament.

As used herein a “pharmaceutical composition” refers to a preparation of the conjugates presented herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions for use in accordance with the present embodiments thus may be formulated in conventional manner using one or more pharmaceutically acceptable carriers comprising excipients and auxiliaries, which facilitate processing of the present conjugates into preparations which can be used pharmaceutically. Proper formulation is dependent upon the route of administration chosen. The dosage may vary depending upon the dosage form employed and the route of administration utilized. The exact formulation, route of administration and dosage can be chosen by the individual physician in view of the patient's condition (see e.g., Fingl et al., 1975, in “The Pharmacological Basis of Therapeutics”, Ch. 1 p. 1).

The pharmaceutical composition may be formulated for administration in either one or more of routes depending on whether local or systemic treatment or administration is of choice, and on the area to be treated. Administration may be done orally, by inhalation, or parenterally, for example by intravenous drip or intraperitoneal, subcutaneous, intramuscular or intravenous injection, or topically (including ophtalmically, vaginally, rectally, intranasally).

Formulations for topical administration may include but are not limited to lotions, ointments, gels, creams, suppositories, drops, liquids, sprays and powders. Conventional pharmaceutical carriers, aqueous, powder or oily bases, thickeners and the like may be necessary or desirable.

Compositions for oral administration include powders or granules, suspensions or solutions in water or non-aqueous media, sachets, pills, caplets, capsules or tablets. Thickeners, diluents, flavorings, dispersing aids, emulsifiers or binders may be desirable.

Formulations for parenteral administration may include, but are not limited to, sterile solutions which may also contain buffers, diluents and other suitable additives. Slow release compositions are envisaged for treatment.

The amount of a composition to be administered will, of course, be dependent on the subject being treated, the severity of the affliction, the manner of administration, the judgment of the prescribing physician, etc.

Compositions of the present embodiments may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a conjugate of the invention formulated in a compatible pharmaceutical carrier may also be prepared, placed in an appropriate container, and labeled for treatment of a medical condition which is associated with the polypeptide which forms a part of the conjugate.

According to further embodiments of the any of the compositions, methods and uses presented herein, the conjugates of the present invention can be combined with other active ingredients.

As discussed hereinabove, PEGylation of polypeptide drugs can impart several significant pharmacological advantages over the unmodified form thereof, by increasing the molecular weight of the polypeptide and providing some degree of protection thereto, which also improves the drug solubility, reduces dosage frequency without diminished efficacy with potentially reduced toxicity, extends circulating life, increases drug stability and enhances protection from proteolytic degradation. The PEGylation process also opens other avenues, such as new delivery formats and dosing and administration regimens.

For example, interferons (e.g., as interferon beta-1a and interferon beta-1b) which are PEGylated via a methionine side chain, can be used to treat many diseases which are treatable by these interferons, such as multiple sclerosis and the relapsing-remitting form of multiple sclerosis, respectively.

Interferons belonging to the interferon alpha-2 family, which are PEGylated via a methionine side chain, can be used to treat many diseases which are treatable by these interferons, such as Kaposi's sarcoma, anogenital warts, hepatitis B and C and anti-HIV.

Erythropoietin (EPO), PEGylated via a methionine side chain, can be used to treat many diseases such as anaemia, neurodegenerative diseases and chronic kidney diseases, and as adjuvant therapy in the treatment of cancer.

Granulocyte colony-stimulating factor (G-CSF), PEGylated according to the present embodiments, can be used to accelerate recovery from neutropenia after chemotherapy; to increase the number of hematopoietic stem cells in the blood of the donor before collection by leukapheresis for use in hematopoietic stem cell transplantation; and to treat heart degeneration.

Human growth hormones (GH) are known to have a beneficial effect on many human diseases. A human GH PEGylated according to the present embodiments, can therefore be used to treat diseases such as, for example, Turner syndrome, chronic renal failure, Prader-Willi syndrome, intrauterine growth retardation, severe idiopathic short stature, AIDS, short bowel syndrome, remission of multiple sclerosis, aging in older adults, obesity, fibromyalgia, Crohn's disease and ulcerative colitis, and can also be used for other purposes such as bodybuilding or athletic enhancement. Non-human growth hormones, modified according to the present embodiments, can be used to treat farm animals or to modify their productivity, such as to increase milk production in cattle.

Follicle-Stimulating Hormone (FSH) is involved in controlling the menstrual cycle and the production of eggs by the ovaries. The amount of FSH varies throughout a woman's menstrual cycle and peaks just before ovulation. In men, FSH is involved in controlling the production of sperm, and its level typically remains constant. Abnormally low level of FSH can result in failure of gonadal function (hypogonadism), which is typically manifested in males as failure in production of normal numbers of sperm, and cessation of reproductive cycles in females. Conditions which are associated with abnormally low level of FSH include infertility, polycystic ovarian syndrome (POS), POS combined with obesity hirsutism and infertility, Kallmann syndrome, hypothalamic suppression, hypopituitarism, hyperprolactinemia, gonadotropin deficiency, and gonadal suppression.

Hence, according to another aspect of embodiments of the invention, there is provided a method of treating a medical condition which is treatable by a polypeptide that at least one methionine residue. According to some embodiments of the present invention, the method is effected by administering to a subject in need thereof a therapeutically effective amount of one or more of the PEGylated conjugates, as described hereinabove.

As used herein, the terms “treating” and “treatment” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the phrase “therapeutically effective amount” describes an amount of the composite being administered which will relieve to some extent one or more of the symptoms of the condition being treated.

The method of treatment, according to some embodiments of the invention, may include the administration of an additional therapeutically active agent.

Medical conditions which are treatable by polypeptides that have at least one methionine residue include, for some non-limiting examples, Kaposi's sarcoma, anogenital warts, hepatitis B and C, AIDS, anaemia, neurodegenerative diseases and chronic kidney diseases, cancer as adjuvant therapy, neutropenia after chemotherapy; heart degeneration, Turner syndrome, chronic renal failure, Prader-Willi syndrome, intrauterine growth retardation, severe idiopathic short stature, short bowel syndrome, remission of multiple sclerosis, aging in older adults, obesity, fibromyalgia, Crohn's disease and ulcerative colitis, infertility, polycystic ovarian syndrome (POS), POS combined with obesity hirsutism and infertility, Kallmann syndrome, hypothalamic suppression, hypopituitarism, hyperprolactinemia, gonadotropin deficiency and gonadal suppression.

Since the aforementioned polypeptides are in a form of a PEGylated conjugate, the administration can be effected, for example, orally, by intravenous injection, by subcutaneous injection or topically.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions; illustrate the invention in a non limiting fashion.

### Materials and Methods

The human recombinant protein samples interferon-beta-1b, interferon-beta-1a, G-CSF and FSH were obtained from InSight Biopharmaceuticals, Israel.

Recombinant human erythropoietin (rh-EPO), marketed as NeoRecormon® was purchased from Hoffman-LaRoche (lot #MH69265)

Recombinant human interferon-alpha-2a (rh-INF-α2a) was purchased from Amoytop Biotech, China.

Recombinant human growth hormone (rh-GH) (cat#CYT-202) was purchased from Prospec-Tany, Israel

30 kDa Methoxy polyethylene glycol N-ethyl-2-iodo-acetamide (cat#6293) was purchased from Biovectra DCL, Canada.

30 kDa Methoxy polyethylene glycol ethylamine (cat#6234) was purchased from Biovectra DCL, Canada

Endoproteinase Lys-C (cat #P3428) was purchased from Sigma-Aldrich

Empigen BB detergent (cat #45165) was purchased from Sigma-Aldrich

Tosyl phenylalanyl chloromethyl ketone (TPCK) treated trypsin (cat #3740) was purchased from Worthington Biochemical Corporation.

HPLC was performed on an Agilent 1200 and a Waters 2695 instrument.

Determination of protein concentration was performed on a Nanodrop ND1000 spectrophotometer

MALDI-TOF and ESI mass spectrometry was performed at the Weizmann Institute, Israel

### Example 1

### PEGylation of Methionine Containing Proteins—General Procedure

PEGylation of recombinant and/or native proteins which exhibit at least one unmodified methionine side chain in their structure is performed with an exemplary PEG moiety such as 30 kDa methoxy polyethylene glycol reagents as follows:

A solution of 2.5 μmol of a methoxy polyethylene glycol reagent in acetate buffer (pH 4) is added to a vial containing a solution of 0.055 μmol protein dissolved in acetate buffer (pH 4). The reaction is stirred in the dark at 25° C. for a time period ranging from about 24 hours to about 200 hours.

The reaction mixture is thereafter diluted with the reaction buffer and loaded onto a chromatographic column which is pre-equilibrated with the reaction buffer, utilizing a FPLC system. The loaded column is washed with the reaction buffer and the unbound fraction is collected. The sample is eluded with an acidic, neutral or alkali solution or saline in the reaction buffer solution and the fractions are collected into tubes.

Alternatively, the reaction mixture is purified by preparative reverse-phase chromatography (RP-HPLC) by injecting the mixture into a Jupiter C4 column with acetonitrile-water and 0.2% TFA as mobile phase. The fraction containing the PEGylated protein is collected and concentrated by centrifugation of the media through a filter and by ultrafiltration on Nanosep 3K (Pall).

An SDS-PAGE is run under non-reducing conditions in order to avoid cleavage of the methionine sulfonium bonds by mercaptoethanol and other harsh reagents as shown previously [Naider, F and Bohak, Z, Biochemistry, 11, 3208, 1972]. The samples are subjected to non-reducing SDS-PAGE 4-12% bis-tris gel with MOPS buffer. The separation gel is run for 50 minutes at 200 V, and thereafter the gel is exposed for 15 minutes with fixing solution and stained, after washing the gel for 1 hour with GelCode Blue. The separation gel is thereafter destained for 4 hours, and photographed. The separation gel is incubated in a solution of barium chloride in water (5%), according to the procedure described in Basu et al., Bioconjug. Chem., 17, 618, 2006. After several rinses with water, the gel is stained with iodine solution (0.1 N Titrisol, Merck) for 5 minutes followed by destaining with several replacements of the water.

### Example 2

### PEGylation of Recombinant Human Interferon-beta-1b (rh-IFN-β1b) with 30 kDa Methoxy Polyethylene Glycol N-ethyl-2-iodo-acetamide

Interferon beta-1b, marketed as BETASERON® by Berlex Corporation, is produced in modified E. coli strands and used to treat multiple sclerosis typically by subcutaneous injection, and has been shown to slow the advance of the affliction as well as reduce the frequency of attacks.

The PEGylation reaction was performed using PEG-iodoacetamide, as depicted in Scheme 1 below.

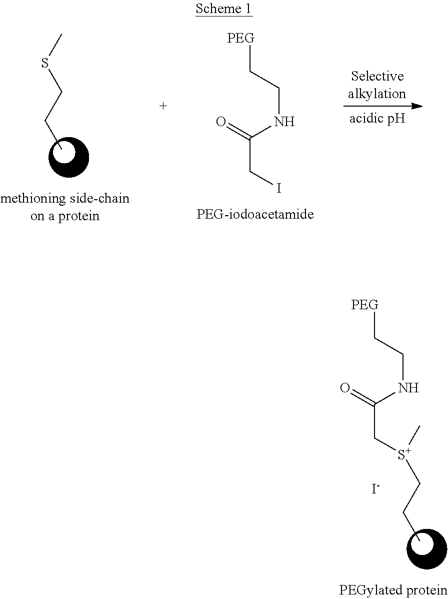

A solution of 75 mg (2.5 μmol) 30 kDa methoxy polyethylene glycol N-ethyl-2-iodo-acetamide in 0.15 ml of phosphoric acid buffer solution (0.1 M, pH 2.52) was added to a vial containing a solution of 0.975 mg (0.055 μmol) rh-IFN-β1b in 1 ml phosphoric acid buffer solution (0.1 M, pH 2.52), and the reaction was stirred in the dark at 25° C. for 1 week (168 hours).

Purification of the reaction mixture by preparative reverse-phase chromatography was performed according to the general procedure presented hereinabove. Briefly, 60 μl of the mixture was injected into a Jupiter C4 column with acetonitrile-water and 0.2% TFA as mobile phase. The fraction of the PEGylated protein was collected at a retention time of 28.9 minutes.

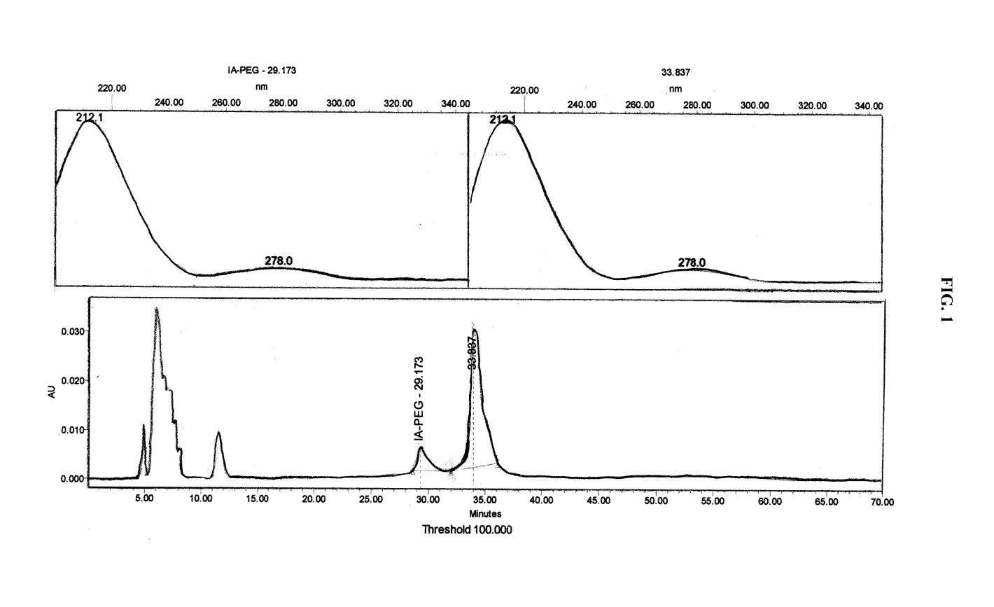

FIG. 1 presents an reverse-phase HPLC (RP-HPLC) chromatogram of the PEGylation reaction products of recombinant human interferon-beta-1b and 30 kDa methoxy polyethylene glycol N-ethyl-2-iodo-acetamide, obtained using a photo-diode array set at 280 nm, an injection volume of 40 μl, total run-time of 70 minutes and 0.2% TFA in water/acetonitrile as a mobile phase, showing a peak having a retention time of 29.17 minutes and a peak corresponding to interferon-beta-1b having a retention time of 33.84 minutes. This fraction was concentrated to 100 μl by speed-vac and by ultrafiltration on Nanosep 3K (Pall), and analyzed in a non-reducing SDS-PAGE.

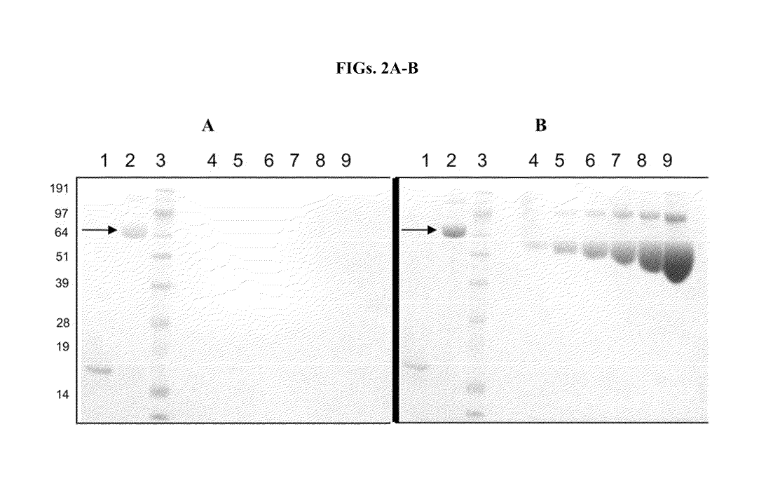

FIGS. 2A-B present color images of a non-reducing SDS-PAGE gel slab in which the PEGylation reaction product of recombinant human interferon-beta-1b and 30 kDa methoxy polyethylene glycol N-ethyl-2-iodo-acetamide (as isolated by RP-HPLC) and the starting materials of the PEGylation reaction were run and stained with Coomassie Blue (FIG. 2A) and subsequently with iodine (FIG. 2B), wherein an un-PEGylated sample of interferon-beta-1b was run in lane 1; the isolated peak by RP-HPLC was run in lane 2; molecular weight markers were run in lane 3; and a series of samples of 30 kDa methoxy polyethylene glycol N-ethyl-2-iodo-acetamide (the PEGylation reaction product) at various concentrations (0.078 μg, 0.3125 μg, 3.125 μg, 6.25 μg, 12.5 μg and 25 μg) which were run in lanes 4, 5, 6, 7, 8 and 9 respectively.

As can be seen in FIGS. 2a-b, interferon-beta-1b appeared as a blue band having a molecular weight of about 18 kDa in the Coomassie blue stained gel (FIG. 2aA, lane 1), and was not stained in brown with iodine (FIG. 2bB, lane 1). The PEG N-ethyl-2-iodo-acetamide reagent at different concentrations stained only with iodine and not by the Coomassie blue, appeared as a brown band having an apparent molecular weight of about 55 kDa (FIG. 2bB, lanes 4, 5, 6, 7, 8 and 9). The sample collected by RP-HPLC exhibited a new species which is seen as a new band (marked with an arrow in FIGS. 2aA-bB, lane 2) having an apparent molecular weight of about 70 kDa, a size which is the sum of the molecular weight of interferon-beta-1b and the molecular weight of the PEG moiety. This band was stained by both with Coomassie blue and iodine.

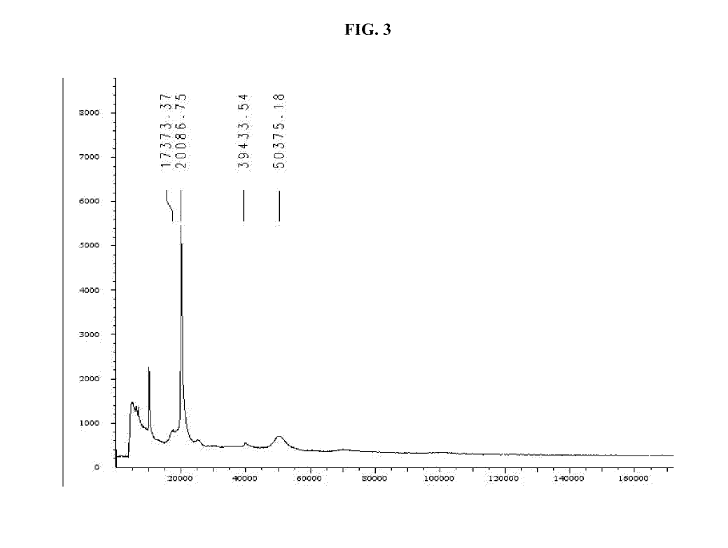

The fraction of the PEGylated interferon-beta-1b, collected by RP-HPLC, was further analyzed by MALDI-TOF mass spectrometry using a 2,4-dihydroxybenzoic acid matrix. FIG. 3 presents a MALDI-TOF mass spectrogram of the PEGylation reaction product of recombinant human interferon-beta-1b with 30 kDa methoxy polyethylene glycol N-ethyl-2-iodo-acetamide, using a 2,4-dihydroxybenzoic acid matrix, showing a small peak having an average molecular weight of about 50,000 Da corresponding to the molecular weight of a mono-PEGylated interferon-beta-1b conjugate;

As can be seen in FIG. 3, this analysis revealed the presence of a peak which corresponds to an average molecular weight of about 50 kDa, corroborating that a mono-PEGylated interferon-beta-1b was formed. Surprisingly, the main peak corresponds to an average molecular weight of about 20 kDa, corresponding to interferon-beta-1b which was not visible in the SDS-PAGE. A possible explanation to this discrepancy may be that the methionine sulfonium bond of the PEGylated interferon-beta-1b severed during the mass spectrometry experiment leading back to the starting material. A further analysis of this phenomenon is delineated hereinafter (see, Example 4).

The fact that the PEGylation occurred at pH 2.5 is indicative of the involvement of the thiomethoxy group of methionine in the chemical modification, since methionine is the only amino acid that is chemically reactive in alkylation reactions at this pH.

### Example 3

### Synthesis of 30 kDa Methoxy Polyethylene Glycol N-ethyl-(4-bromomethyl)-benzamide

Oxalyl chloride (14.4 mg, 0.114 mmol) and 1 drop of DMF were added to a mixture of 4-(bromomethyl)benzoic acid (8.2 mg; 0.038 mmol) dissolved in 0.5 ml dry THF and cooled to 0° C. The mixture was stirred for 2 hours and thereafter the solvent was evaporated under reduced pressure to give a yellowish product. This product was dissolved in 1 ml of dry dioxane and added to a mixture of 30 kDa PEG-ethylamine (190 mg; 0.0063 mmol) and triethylamine (22.9 mg, 0.2 mmol) in 1.5 ml of dioxane. The resulting mixture was stirred for 16 hours at 25° C. and a white suspension was formed. After addition of 10 ml of dry ether the mixture was filtered and the white solid residue was triturated with dry ether and thereafter dried to afford 187.5 mg of product at an overall yield of 97%.

The reactivity of the PEG-benzyl bromide for thiol groups found in cysteine side-chains and methylsulfanyl groups found in methionine side-chains, was evaluated at about 90% by an indirect Ellman's test assay, performed according to a published procedure [Morpurgo and Veronese, Methods Mol. Biol, 288, 45-70, 2004]).

### Example 4

### PEGylation of Recombinant Human Interferon-beta-1b (rh-IFN-β1b) with 30 kDa Methoxy Polyethylene Glycol N-ethyl-(4-bromomethyl)-benzamide

In order to study the efficiency of other PEGylation reagents, the reaction presented hereinabove with PEG-iodoacetamide was performed using PEG-benzyl bromide as a PEGylation reagent, as depicted in Scheme 2 below.

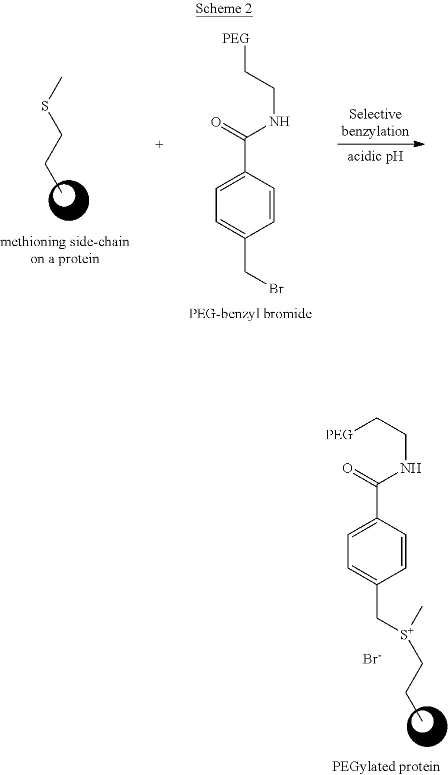

Methoxy polyethylene glycol N-ethyl-(4-bromomethyl)-benzamide (30 kDa, 27.7 mg, 0.92 μmol) was added to a solution of rh-IFN-β1b (3.70 mg, 0.18 μmol) in phosphoric acid (0.77 ml, 0.01 M, pH 2.5), and the reaction was incubated in a “head over tail” shaker at 22° C. for 24 hours. Substantial amounts of PEGylated product was formed in 24 hours, indicating the high reactivity of the PEG-benzyl bromide reagent.

Purification of the reaction mixture by preparative reverse-phase chromatography was performed by injecting 50 μl of the mixture into a Jupiter C4 column with acetonitrile-water and 0.2% TFA as mobile phase. The fraction of the PEGylated protein was collected at a retention time of 38 minutes.

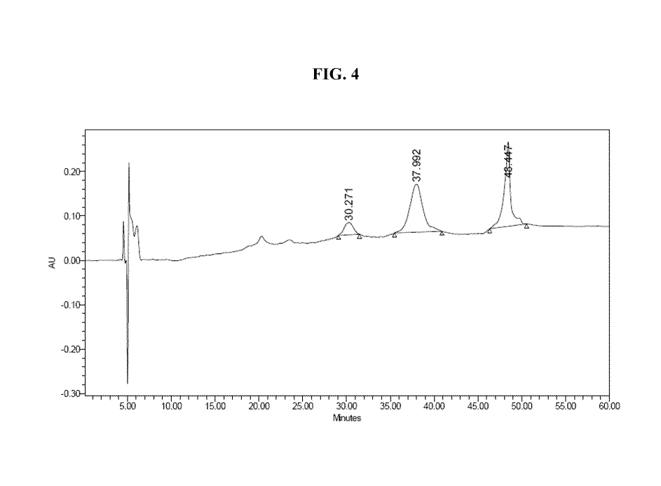

FIG. 4 presents an RP-HPLC chromatogram of the PEGylation reaction product of recombinant human interferon-beta-1b and 30 kDa methoxy polyethylene glycol N-ethyl-(4-bromomethyl)-benzamide, showing a peak having a retention time of 30.27 minutes corresponding to methoxy polyethylene glycol N-ethyl-(4-bromomethyl)-benzamide, a second peak having a retention time of 38 minutes corresponding to the PEGylated protein, and a peak having a retention time of 48.44 minutes corresponding to recombinant human interferon-beta-1b.

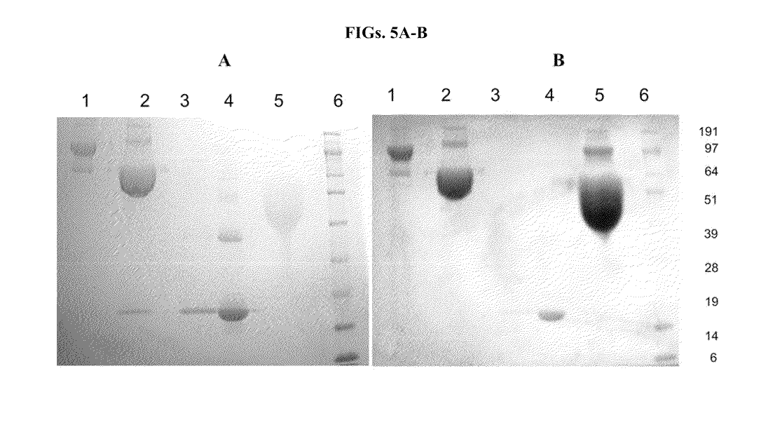

The fractions were concentrated to 100 μl by speed-vac and were analyzed on two non-reducing SDS-PAGE gel slabs and color images of the obtained slabs are presented in FIGS. 5A-B.

FIGS. 5A-B present color images of a non-reducing SDS-PAGE separation gel in which the PEGylation reaction products of recombinant human interferon-beta-1b and 30 kDa methoxy polyethylene glycol N-ethyl-(4-bromomethyl)-benzamide, after collection by RP-HPLC, were run and stained with Coomassie Blue (FIG. 5A) and subsequently with iodine (FIG. 5B), wherein the collected fractions were run in lanes 1, 2 and 3; an un-PEGylated sample of recombinant human interferon-beta-1b was run in lane 4, a 30 kDa methoxy polyethylene glycol N-ethyl-(4-bromomethyl)-benzamide sample was run in lane 5 and molecular weight markers were run in lane 6.

As can be seen in FIGS. 5A-B, lane 4, interferon-beta-1b appeared as an 18 kDa band stained with Coomassie and not with iodine. The PEG N-ethyl-(4-bromomethyl)-benzamide reagent stained only with iodine and not by the Coomassie blue, appeared as a brown band having an apparent molecular weight of about 55 kDa (FIG. 5B, lane 5). It can be observed that fraction 1 collected from RP-HPLC (retention time of about 30 minutes) is mainly a di-PEGylated product since it appeared as a band having a molecular weight of about 120 kDa (which is the sum of interferon-beta-1b and twice the apparent molecular weight of PEG) in the Coomassie stained gel (FIG. 5A, lane 1), and was stained brown with iodine (FIG. 5b, lane 1). In addition, it can be seen that fraction 2 collected from RP-HPLC (retention time of about 38 minutes) is mainly a mono-PEGylated product since it appeared as a band having a molecular weight of about 70 kDa (which is the sum of interferon-beta-1b and the apparent molecular weight of PEG) in the Coomassie stained gel (FIG. 5A, lane 2), and was stained in brown with iodine (FIG. 5B, lane 2). Fraction 3 collected from RP-HPLC (retention time of about 48 minutes) is the non-reacted interferon-beta-1b (FIGS. 5a-b, lane 3) similarly to the interferon-beta-1b standard (FIGS. 5A-B, lane 4). These results indicate that a mono-PEGylated interferon-beta-1b was formed by using a PEG N-ethyl-(4-bromomethyl)-benzamide reagent at acidic conditions.

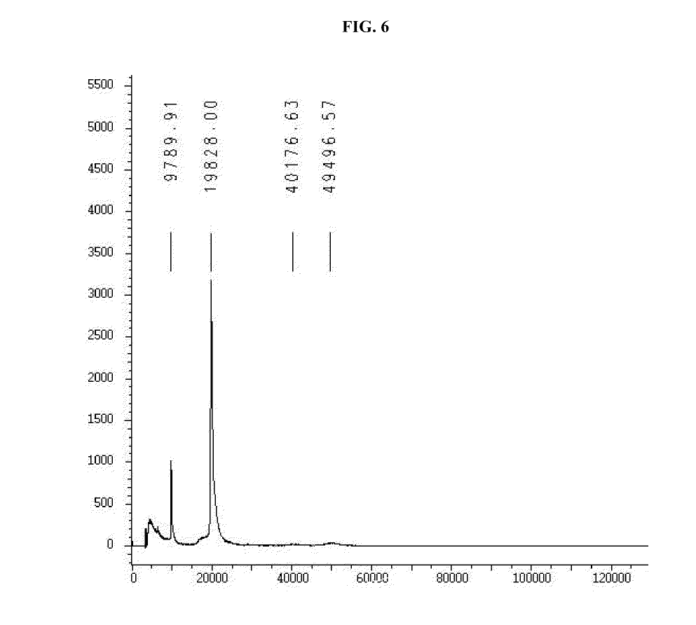

The sample collected by RP-HPLC that was assigned as the PEGylated protein was analyzed by MALDI-TOF mass spectrometry. FIG. 6 presents a MALDI-TOF mass spectrogram of the purified PEGylation product of recombinant human interferon-beta-1b with 30 kDa methoxy polyethylene glycol N-ethyl-(4-bromomethyl)-benzamide, using a 2,4-dihydroxybenzoic acid matrix.

As can be seen in FIG. 6, a peak corresponding to species of an average molecular weight of about 50 kDa was detected, as in the case of PEGylated interferon beta-1b produced with 30 kDa PEG-iodoacetamide, corroborating that a mono-PEGylated interferon-beta-1b was formed. Surprisingly, the main peak with an average molecular weight of about 20 kDa, was identified as belonging to interferon-beta-1b, a species which was not detectable in the SDS-PAGE. As already postulated in the case of PEGylated interferon beta-1b from PEG-iodoacetamide, a possible explanation to this discrepancy was that the relatively labile methionine sulfonium bond of the PEGylated interferon-beta-1b was disrupted during the mass spectrometry experiment, leading back to the starting material. Decomposition of proteins bearing alkylated methionines has been reported to occur under mass spectrometry conditions [Bykova, N V et al., Anal. Chem., 78, 1093-103, 2006]. A likely reaction mechanism for decomposition of the sulfonium salt in the source of the mass spectrometer is the Hoffman elimination reaction. Derivatization of methionine followed by this decomposition yields a net decrease of 48 Da from the mass of the starting protein (see Scheme 3 below). Careful analysis of the MW of the main peak in the MALDI-TOF spectrum shows that the MW of 30 kDa PEG-interferon beta-1b after decomposition is 51 Da less than the theoretical MW value of interferon beta-1b (19,879 Da). This net decrease is similar to the MW of the Hofmann elimination product (decrease by 48 Da) and may be in the inaccuracy range of the MALDI-TOF measurement.

Scheme 3 presents a typical methionine side-chain modification with iodoacetamide undergoing collision-induced dissociation (CID) fragmentation in mass spectrometry by Hoffman elimination.

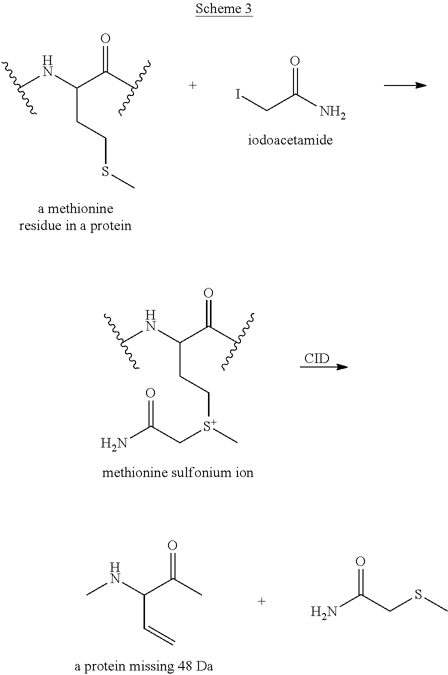

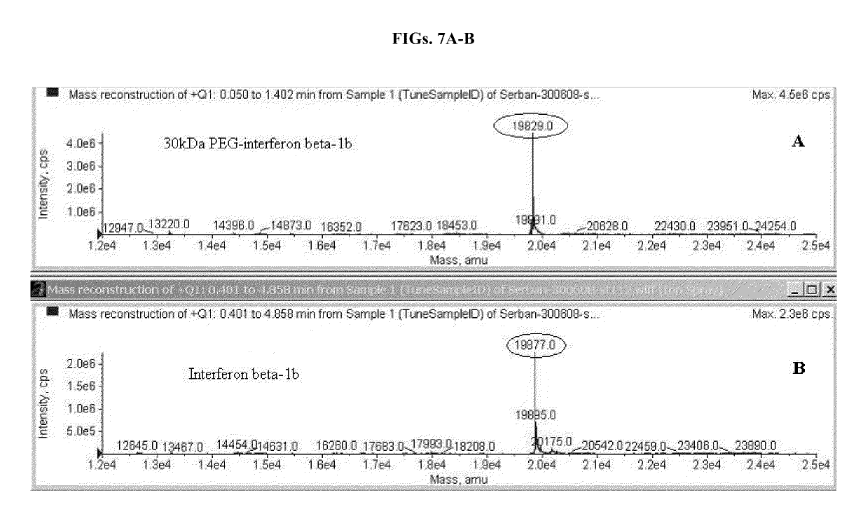

The 30 kDa PEGylated interferon-beta-1b was also analyzed by the more accurate electrospray ionization (ESI) mass spectrometry. FIGS. 7A-B present ESI mass spectrograms of the purified PEGylation product of recombinant human interferon-beta-1b with 30 kDa methoxy polyethylene glycol N-ethyl-(4-bromomethyl)-benzamide (FIG. 7A) and recombinant human interferon-beta-1b (FIG. 7B).

As can be seen in FIGS. 7A-B, no molecular peak of a 30kDa PEG-interferon beta-1b conjugate was observed (about 50 kDa), as in the case of the MALDI-TOF mass spectrometry analysis, and the main peak was due to the Hoffman elimination described hereinabove, resulting in a mass of 19,829 Da.

The measured molecular mass of interferon beta-1b by ESI was 19,877 Da (see FIG. 7B). Therefore, the difference in mass between decomposed 30kDa PEG-interferon beta-1b by Hoffman elimination and interferon beta-1b is exactly 48 Da. which is very specific to alkylated methionines (see Scheme 3).

Since no other cases of decomposition of PEGylated proteins have been described, both mass spectrometry techniques indicate very strongly that the PEGylation at acidic pH occurred via the methionines as was targeted.

### Example 5

### Peptide Mapping of PEGylated Recombinant Human Interferon-beta-1b

In order to study the effect of PEGylation and to verify the PEGylation site along the polypeptide chain, PEGylated recombinant human interferon-beta-1b (rh-IFN-β1b) was subjected to protease digestion, and the breakup products were compared to those obtain from the un-PEGylated peptide by a method known as peptide mapping.

Peptide mapping was performed according to Johnson-Jackson, D et al. [WO 08/020968]. Recombinant-human-IFN-β1b was prepared at a concentration of 0.5 mg/ml in 2 mM aspartic acid. Empigen BB (30%, 1.3 μl) was added to rh-IFN-β1b, and the pH was adjusted to 7 using Tris-HCl pH 7 (1 M, 62.5 μl). The digest was started by addition of 1 μl Lys-C (1 mg/ml), and the reaction mixture was incubated at 37° C. with a “head-over-tail” shaking for 4 hours. Thereafter, another aliquote of 1 μl of Lys-C was added and sample was incubated for an additional 24 h. To quench the digest, 13 μl of 8 M guanidine HCl was added to the reaction mixture.

Peptide fragments were resolved by HPLC chromatography by injecting 50 μl of the mixture into a Jupiter C4 column with acetonitrile-water and 0.1% TFA as mobile phase.

In a control reaction in which the digested peptides were reduced at the end of the incubation, 0.5 μl of 1 M DTT was added to 90 μl of the reaction mixture, and incubation was performed for 45 minutes at 37° C.

Peptide mapping of PEGylated rh-IFN-β1b was performed simultaneously and in the same manner as peptide mapping of rh-IFN-β1b described above.

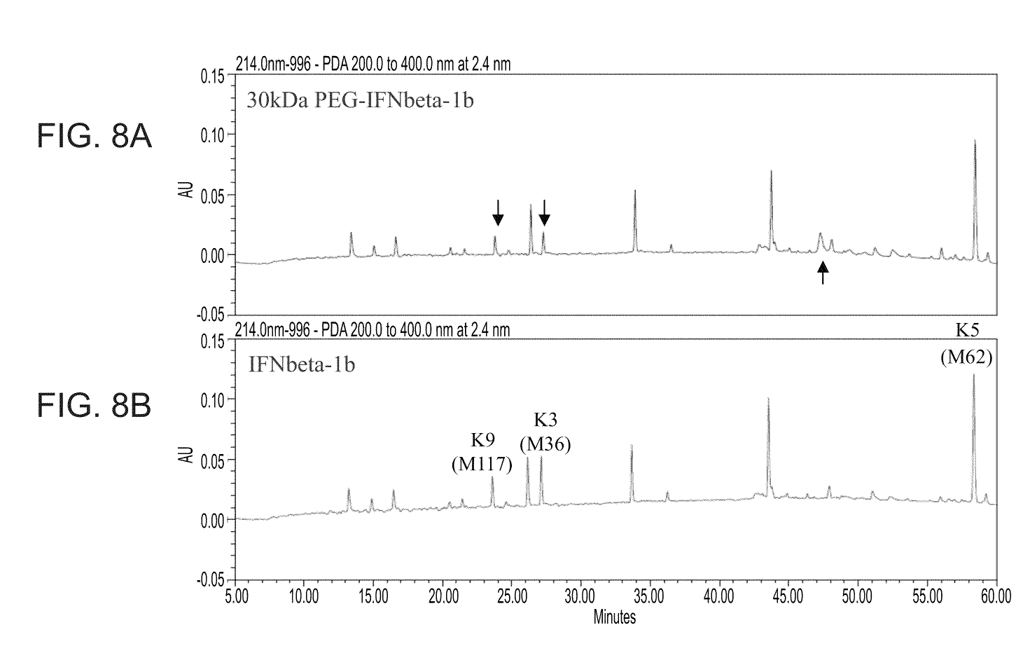

FIGS. 8A-B present RP-HPLC chromatograms showing the peptide maps of purified PEGylation product of recombinant human interferon-beta-1b with 30 kDa methoxy polyethylene glycol N-ethyl-(4-bromomethyl)-benzamide (FIG. 8A), and of recombinant human interferon-beta-1b (FIG. 8B), as obtained by applying peptide digestion using Lys-C.

As can be seen in FIG. 8B, the peptide map that was obtained by Lys-C digestion of interferon beta-1b is similar to the published peptide map of the commercial interferon beta-1b under similar conditions [Lin, L S, Kunitani, M G and Hora, M S, Interferon-beta-1b (BETASERON®): A model for hydrophobic therapeutic proteins in Formulation, characterization and stability of protein drugs, Pearlman, R and Wang, Y J (Eds.), Plenum Press, NY, pp. 275-301, 1996 and Johnson-Jackson, D et al., WO 08/020968], which allows assigning the peaks to the corresponding peptides. When peptide mapping of interferon beta-1b by Lys-C was performed under reducing conditions the retention time of the peptide containing the single S—S bond (K2-12) changed from 43.07 minutes to 43.52 minutes (K12) and a new peak was formed with a retention time of 33.55 minutes corresponding to K2 according to Johnson-Jackson, D et al., WO 08/020968 (data not shown).

As can be seen in FIG. 8A, the peptide map of PEGylated interferon beta-1b, which is probably a mixture of PEGylated isoforms, digested by Lys-C is similar to the peptide map of interferon beta-1b digested by Lys-C, but some differences are observed (see FIG. 8B). The intensity of mainly two methionine containing peptides, K9 and K3, decreased in the digested PEGylated protein as compared to the corresponding peaks in interferon beta-1b digests. The intensity of the third peptide that contains methionine (K5) was the same in both peptide maps. In addition, in the case of the digested PEGylated protein a new peptide was formed having a retention time of 47.5 minutes. From comparison of both peptide maps it may be concluded that a selective PEGylation occurred via methionine amino acids M117 and M36 and not via M62. These results are in agreement with the surface accessibility of these methionines as observed in the crystal structure of interferon beta-1a [Karpusas, M et al., Proc. Natl. Acad. Sci., 94, 11813, 1997] and with the observed chemical susceptibility of interferon beta-1a after oxidation and alkylation [Orru, S et al., Biol. Chem., 381, 7, 2000].

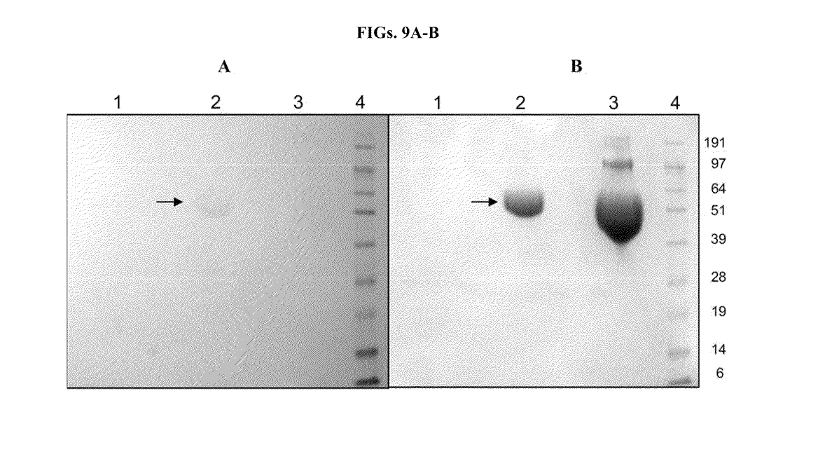

The peptide K3 and the new peptide, having a retention time of 47.5 minutes, which were formed after the peptide Lys-C digestion of the purified PEGylation product of recombinant human interferon-beta-1b with 30 kDa methoxy polyethylene glycol N-ethyl-(4-bromomethyl)-benzamide with Lys-C, were collected by RP-HPLC and subjected to electrophoresis, and the results are shown in FIGS. 9A-B.

FIGS. 9A-B present color images of a non-reducing SDS-PAGE separation gel in which the peptides which were formed after peptide Lys-C digestion of the purified PEGylation product of recombinant human interferon-beta-1b with 30 kDa methoxy polyethylene glycol N-ethyl-(4-bromomethyl)-benzamide were run and stained with Coomassie Blue (FIG. 9A) and subsequently with iodine (FIG. 9B), wherein the collected fractions of peptide K3 and the new formed peptide with a retention time of 47.5 minutes were run in lanes 1 and 2 respectively; a 30 kDa methoxy polyethylene glycol N-ethyl-(4-bromomethyl)-benzamide sample was run in lane 3 and molecular weight markers were run in lane 4.

As can be seen from FIGS. 9A-B, peptide K3 (lane 1) may not be seen on the gel, as expected due to its low MW. In contrast, the new peak (FIG. 9, lane 2) appeared with a MW similar to the PEG reagent, as expected for a PEGylated peptide. This band stained both with Coomassie and iodine. This evidence indicates that the new peak that appeared in the peptide mapping with Lys-C is the newly PEGylated peptide.

LC/MS/MS analysis of the content of the new peak (FIG. 9, lane 2) has shown that it contains molecular masses that correspond to peptides K3-48 Da (MW=1457.7) and K9-48 Da (MW=879.5) and fragmentations of 44 Da which are typical for the disrupted PEG chain. This is an additional evidence that PEGylation of rh-IFN-β1b with 30 kDa methoxy polyethylene glycol N-ethyl-(4-bromomethyl)-benzamide leads to the formation of only two mono-PEGylated isoforms via methionines M117 and M36.

### Example 6

### Antiviral Activity of 30kDa PEG-Interferon Beta-1b Versus BETASERON®

The antiviral activity of the conjugate of 30 kDa PEG-Interferon beta-1b, an exemplary PEGylated active cytokine according to some embodiments of the present invention, was determined by the capacity of the PEGylated cytokine to protect human amnion WISH cells against vesicular stomatitis virus (VSV) induced cytopathic effects [Rubinstein, M. et al. [J. Virol., 37, 755-758, 1981].

Lyophilized 30 kDa PEG-Interferon-beta-1b conjugate was reconstituted in 2 ml formulation buffer (5 mM aspartic acid, 5% mannitol, pH 4) to afford a solution at a concentration of 40 μg/ml.

The introduction of 30 kDa PEG-Interferon-beta-1b to the WISH cells had a specific anti-viral activity of approximately 85% compared to that of BETASERON®, indicating that the PEGylation of the cytokine did not reduce its bioactivity. This result is extraordinarily positive for a PEGylated cytokine as other known and commercially available PEGylated cytokines, such as PEGASYS®, are said to retain only 7% of the original bioactivity of the non-PEGylated cytokine [Bailon, P et al., Bioconj. Chem., 12, 195-202, 2001].

### Example 7

### Pharmacokinetic Profile of 30 kDa PEG-Interferon-beta-1b

The pharmacokinetic profile of 30 kDa PEG-Interferon-beta-1b, an exemplary PEGylated active cytokine according to some embodiments of the present invention, was compared to that of a commercially available for of the cytokine, namely BETAFERON®. The study was performed by monitoring the cytokine concentration in the plasma of rats following intravenous administration of the two drugs.

Lyophilized 30 kDa PEG-Interferon-beta-1b was reconstituted in formulation buffer (5 mM aspartic acid, 5% mannitol, pH 4), affording a solution of 1 mg/ml of the PEGylated cytokine. BETAFERON® (250 μg in each vial) was dissolved in water for injection to afford a solution of 1 mg/ml. The test compounds were administered intravenously through the cannula of three cannulated rats each at a single dose of 0.4 mg/kg.

For both test compounds, the blood samples were collected from the rats after 0.083, 0.25, 0.5, 1, 2, 4, 7 and 24 hours. In the case of 30 kDa PEG-Interferon-beta-1b, additional blood samples were collected after 48, 72 and 96 hours. The plasma was separated by centrifugation at 5000 rpm at 4° C. for 5 minutes, transferred to plastic vials, frozen on dry ice and stored at −80° C.

Analysis of samples was performed using Human IFN-β Elisa kit (Fujirebio Inc., cat #KAC1201), according to the protocol provided with the kit. The concentration of 30 kDa PEG-Interferon-beta-1b and BETAFERON® in the blood samples were calculated from their respective standard curves.

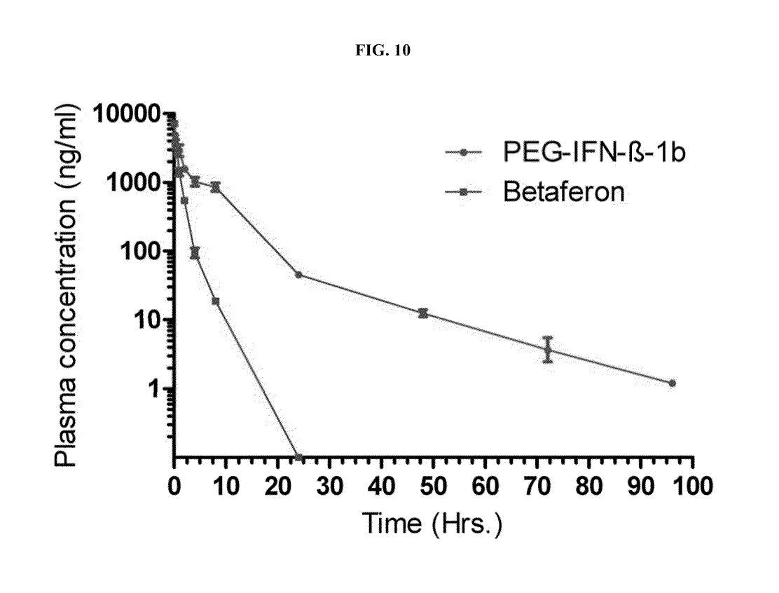

FIG. 10 presents a comparative plot of the concentration of interferon-beta-1b (INF-β1b) in plasma of rats as a function of time, following intravenous administration of 0.4 mg/kg of 30 kDa PEG-Interferon beta-1b (in red) and BETAFERON® (in green), showing the pharmacokinetic profile of the two drugs and the remarkable longer pharmacologic range of the PEGylated interferon according to some embodiments of the present invention.

As can be seen in FIG. 10, the concentration of BETAFERON® in blood decreases very rapidly and 24 hours after administration no drug was detected (half-life is 1 hour). In sharp contrast, 24 hours after administration of the 30 kDa PEG-Interferon-beta-1b, substantial amounts thereof were detected markedly in blood and even after 96 hours traceable amounts (1.2 ng/ml) were still detected (half life is 10.2 hours). The 10-fold longer half-life of PEGylated INF-β1b as compared to BETAFERON® is similar to other PEG-INF-β1b molecules that are PEGylated through sites other than methionine sulfur [Basu et al., Bioconjug. Chem., 17, 618, 2006].

### Example 8

### PEGylation of Recombinant Human Interferon-beta-1a (rh-IFN-β1a)

Recombinant human interferon beta-1a (rh-IFN-β1a), marketed as Avonex® by Biogen Idec and Rebif® by Serono, is produced by mammalian cells and used to treat multiple sclerosis (MS) typically by subcutaneous injection, and has been shown to have about a 30% to 35% reduction in the rate of MS relapses, and to slow the progression of disability in MS patients.

A solution of 11.2 mg (0.38 μmol) 30 kDa methoxy polyethylene glycol N-ethyl-2-iodo-acetamide in 0.07 ml acetate buffer (50 mM, pH 4) was added to a vial containing a solution of 0.168 mg (0. 0075 μmol) rh-IFN-β1a in 0.28 ml acetate buffer (50 mM, pH 4). The reaction was stirred in the dark at 25° C. for 144 hours.

The reaction mixture (350 μl) was diluted with a solution of sodium acetate (0.07 ml, 20 mM, pH 4) and the mixture was loaded onto a Fractogel COO⁻ column (0.5 cm×5 cm; by Merck) which was pre-equilibrated with sodium acetate solution (20 mM, pH 4), utilizing an AKTA FPLC system. The loaded column was washed first with sodium acetate solution (50 ml, 20 mM, pH 4) and second with phosphate buffer with 10% propylene glycol (50 ml, 20 mM, pH 7), and the unbound fraction was collected. The elution was done with phosphate buffer with 10% propylene glycol and 1 M NaCl (20 mM, pH 7), and the fractions were collected into tubes.

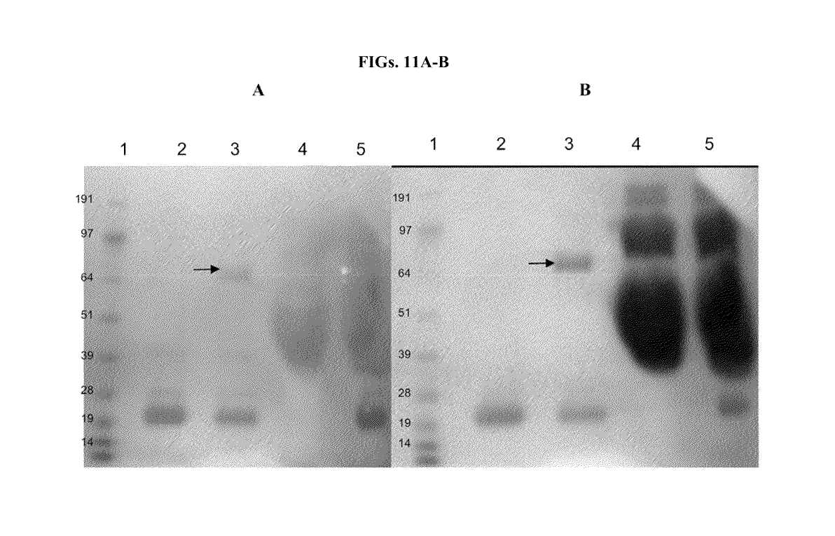

The fractions were analyzed on one non-reducing SDS-PAGE and color images of the obtained slab are presented in FIGS. 11A-B. The gel slab was run and thereafter stained with Coomassie Blue (FIG. 11A), and thereafter the same gel slab was stained with iodine (FIG. 11B). Molecular weight markers were run in lane 1; an un-PEGylated sample of interferon-beta-1a was run in lane 2; the eluted fraction was run in lane 3; the unbound fraction washed with sodium acetate buffer solutions was run in lane 4; and the crude reaction mixture which was loaded on the column was run in lane 5.

As can be seen in FIGS. 11A-B, rh-IFN-β1a appeared as a blue band having a molecular weight of about 22 kDa in the Coomassie blue stained gel (FIG. 11A, lanes 2, 3 and 5), whereas an un-PEGylated sample thereof was not stained in brown. In addition to the 22 kDa band in lane 3, wherein the eluted fraction was run, a new band appeared having an apparent molecular weight of about 75 kDa which is the sum of interferon-beta-1a and the apparent molecular weight of 30 kDa PEG. This band was stained both with Coomassie and iodine solution.

These results clearly show that a mono-PEGylated interferon-beta-1a was afforded.

### Example 9

### PEGylation of Recombinant Human Interferon-alpha-2a (rh-IFN-α2a) with 30 kDa Methoxy Polyethylene Glycol N-ethyl-2-iodo-acetamide

Interferon alpha-2a, marketed as Roferon®-A by Hoffmann-Laroche, is produced in modified E. coli strands. The PEGylation reaction thereof was performed using PEG-iodoacetamide, as depicted in Scheme 1 hereinabove.

A solution of 23.2 mg (0.78 μmol) 30 kDa methoxy polyethylene glycol N-ethyl-2-iodo-acetamide in 0.1 ml of acetate buffer solution (pH 4) was added to a vial containing a solution of 0.3 mg (0.015 μmol) rh-IFN-α2a in 0.3 ml acetate buffer solution (pH 4), and the reaction was stirred in the dark at 25° C. for 5 days (120 hours).

The reaction mixture (400 μl) was diluted with a solution of sodium acetate (1.6 ml, 20 mM, pH 4) and the mixture was loaded onto a Source 15-S column (5 mm×100 mm) which was pre-equilibrated with sodium acetate solution (20 mM, pH 4), utilizing an AKTA FPLC system. The loaded column was washed with sodium acetate solution (60 ml, 20 mM, pH 4), and the unbound fraction was collected. The elution was done with a NaCl solution (0.5 M) in sodium acetate solution (20 mM).

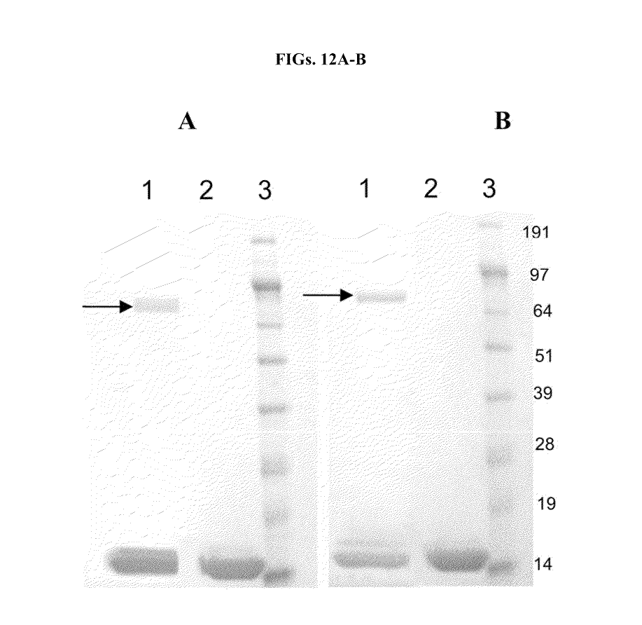

The fractions were analyzed by SDS-PAGE and color images are presented in FIGS. 12A-B.

FIGS. 12A-B present color images of a non-reducing SDS-PAGE separation gel in which the PEGylation reaction products of recombinant human interferon-alpha-2a and 30 kDa methoxy polyethylene glycol N-ethyl-2-iodo-acetamide, which were eluted on a Source 15-S column, were run and stained with Coomassie Blue (FIG. 12A) and iodine (FIG. 12B), wherein the fraction eluted with NaCl solution was run in lane 1; an un-PEGylated sample of interferon-alpha-2a was run in lane 2, and molecular weight markers were run in lane 3; showing the PEGylated protein (marked with an arrow), and an increase of about 50 kDa in the apparent molecular weight of the

PEGylated protein as compared to the un-PEGylated protein.

As can be seen in FIGS. 12A-B, interferon-alpha-2a appeared as a blue band having a molecular weight of about 16 kDa in the Coomassie blue stained gel (FIG. 12A, lanes 1 and 2), whereas an un-PEGylated sample thereof was not stained in brown with iodine (FIG. 12B, lane 2). The eluted sample exhibited a new species which is seen as a new band (marked with an arrow in FIGS. 12A-B, lane 1) having an apparent molecular weight of about 70 kDa, a size which is the sum of interferon-alpha-2a and PEG molecular weight. This band was stained both with Coomassie blue and iodine.

### Example 10

### PEGylation of Recombinant Human Interferon-alpha-2a (rh-IFN-α2a) with 30 kDa Methoxy Polyethylene Glycol N-ethyl-(4-bromomethyl)-benzamide

In order to further study the efficiency of PEG-benzyl bromide as a PEGylation reagent, the PEGylation of interferon alpha-2a, was conducted with PEG-benzyl bromide as depicted in Scheme 2 hereinabove.

Glacial acetic acid (0.5 μl) was added to the solution of rh-IFN-α2a (0.5 ml) to reduce the solution pH to pH 4. This solution was then concentrated to 125 μl corresponding to a protein concentration of 4 mg/ml using Amicon Ultra-4 filters. Protein concentration was determined by Bradford assay. 30 kDa methoxy polyethylene glycol N-ethyl-(4-bromomethyl)-benzamide (3.7 mg, 0. 12 μmol) was added to the above solution of rh-IFN-α2a (0.48 mg, 0.025 μmol) and the reaction was incubated in a head over tail shaker at 25° C. for 24 hours.

Purification of the reaction mixture by preparative reverse-phase chromatography: 20 μl of the mixture were injected to a Jupiter C4 column with acetonitrile-water and 0.2% TFA as mobile phase. Three main peaks that were assigned to be the PEGylated protein were collected at retention time between 20.4-27.2 minutes. These fractions were concentrated by speed-vac and analyzed in a non-reducing SDS-PAGE.

The products of the reaction between interferon-alpha-2a and 30 kDa methoxy polyethylene glycol N-ethyl-(4-bromomethyl)-benzamide were collected by preparative reverse-phase chromatography.

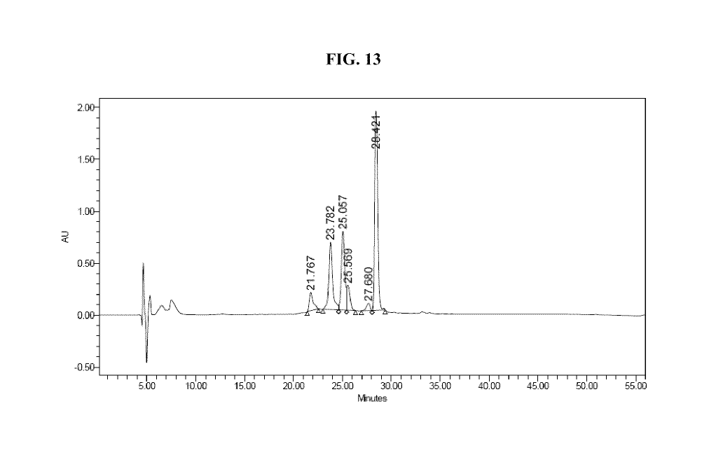

FIG. 13 presents a RP-HPLC chromatogram of the PEGylation reaction products of recombinant human interferon-alpha-2a and 30 kDa methoxy polyethylene glycol N-ethyl-(4-bromomethyl)-benzamide wherein several formed peaks are observed (retention times of 21.77, 23.78, 25.05, 25.57 and 27.42 minutes) and recombinant human interferon-alpha-2a has a retention time of 28.42 minutes.

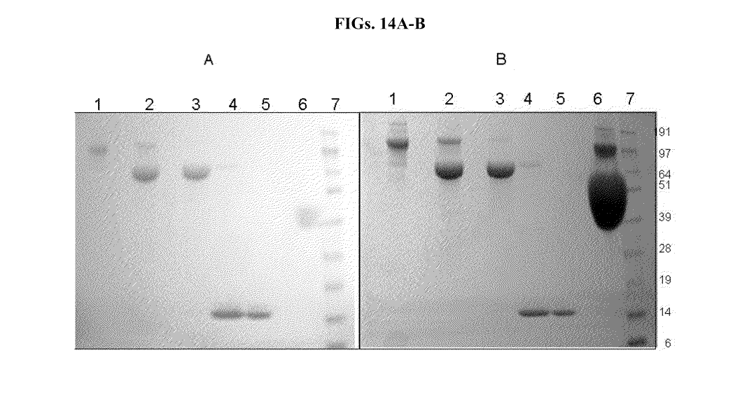

FIGS. 14A-B present color images of a non-reducing SDS-PAGE separation gel in which the PEGylation reaction products of recombinant human interferon-alpha-2a and 30 kDa methoxy polyethylene glycol N-ethyl-(4-bromomethyl)-benzamide, after collection by RP-HPLC, were run and stained with Coomassie Blue (FIG. 14A) and subsequently with iodine (FIG. 14B), wherein the collected fractions were run in lanes 1, 2, 3 and 4; an un-PEGylated sample of recombinant human interferon-alpha-2a was run in lane 5, a 30 kDa methoxy polyethylene glycol N-ethyl-(4-bromomethyl)-benzamide sample was run in lane 6 and molecular weight markers were run in lane 7.

As can be seen in FIGS. 14A-B (lane 5), interferon-alpha-2a appeared as an 18 kDa band stained with Coomassie and not with iodine. The PEG N-ethyl-(4-bromomethyl)-benzamide reagent stained only with iodine and not by the Coomassie blue, appeared as a brown band having an apparent molecular weight of about 55 kDa (FIG. 14B, lane 6). It can be observed that fraction 1 collected from RP-HPLC is mainly a di-PEGylated product since it appeared as a band having a molecular weight of about 120 kDa (which is the sum of interferon-alpha-2a and twice the apparent molecular weight of PEG) in the Coomassie stained gel (FIG. 14A, lane 1), and was stained in brown with iodine (FIG. 14B, lane 1). In addition, it can be seen that fractions 2 and 3 collected from RP-HPLC are mainly mono-PEGylated products (mono-PEGylated isoforms) since they appeared as bands having a molecular weight of about 70 kDa (which is the sum of interferon-beta-1b and the apparent molecular weight of PEG) in the Coomassie stained gel (FIG. 14A, lanes 2-3), and was stained brown with iodine (FIG. 14B, lanes 2-3). Fraction 4 collected from RP-HPLC is the non-reacted interferon-alpha-2a (FIGS. 14A-B, lane 4) similarly to the interferon-alpha-2a standard (FIGS. 14A-B, lane 5).

The results indicate that a mono-PEGylated interferon-alpha-2a was formed by using a PEG N-ethyl-(4-bromomethyl)-benzamide reagent at acidic conditions.

### Example 11

### PEGylation of Recombinant Human Erythropoietin (EPO) with 30 kDa Methoxy Polyethylene Glycol N-ethyl-(4-bromomethyl)-benzamide

Recombinant human erythropoietin (rh-EPO), marketed as NeoRecormon® by Hoffman-Laroche and Procrit® by Johnson and Johnson, is produced by mammalian cells and is used for the treatment of anemia.

Two syringes of NeoRecormon containing 20,000 IU each were used. The buffer was exchanged to 50 mM acetate buffer pH 4 and the protein was concentrated to 80 μl using Amicon Ultra-4 filters. Protein concentration (3 mg/ml) was determined using Nanodrop spectrophotometer. 30 kDa Methoxy polyethylene glycol N-ethyl-(4-bromomethyl)-benzamide (1.2 mg, 0.040 μmol) was added to the above solution of EPO (0.24 mg, 0.008 μmol) and the reaction was incubated in a head over tail shaker at 25° C. for 24 hours.

Purification of the reaction mixture by preparative reverse-phase chromatography was performed by loading 20 μl of the mixture on a Jupiter C4 column with acetonitrile-water and 0.2% TFA as mobile phase. The main peak was assigned to the fraction containing the PEGylated protein, which was collected at retention time between 25-29 minutes. This fraction was concentrated by speed-vac and analyzed in a non-reducing SDS-PAGE.

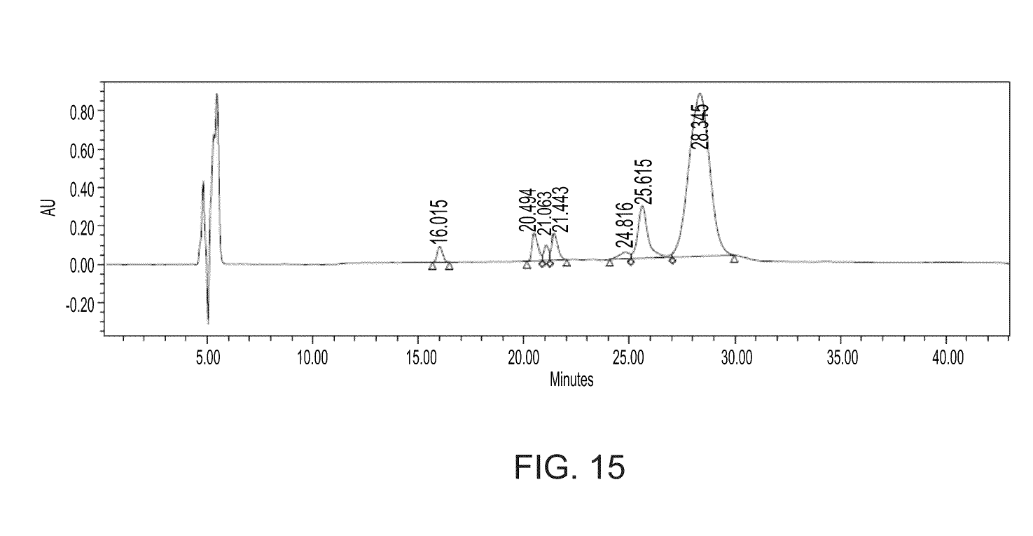

FIG. 15 presents a RP-HPLC chromatogram of the PEGylation reaction products of recombinant human erythropoietin and 30 kDa methoxy polyethylene glycol N-ethyl-(4-bromomethyl)-benzamide wherein a peak is observed with a retention time of 25.62 minutes and recombinant human erythropoietin has a retention time of 28.35 minutes.

The products of the reaction between erythropoietin and 30 kDa methoxy polyethylene glycol N-ethyl-(4-bromomethyl)-benzamide were isolated using preparative reverse-phase chromatography.

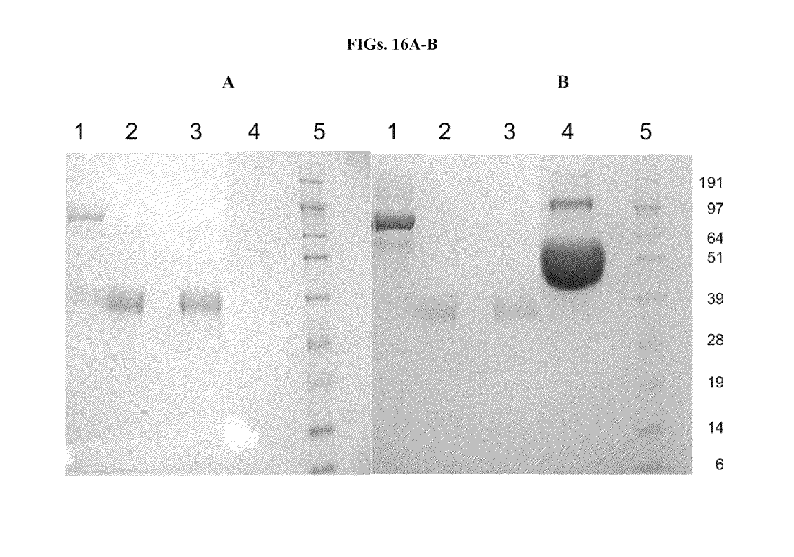

FIGS. 16A-B present color images of a non-reducing SDS-PAGE separation gel in which the PEGylation reaction products of recombinant human erythropoietin and 30 kDa methoxy polyethylene glycol N-ethyl-(4-bromomethyl)-benzamide, after collection by RP-HPLC, were run and stained with Coomassie Blue (FIG. 16A) and subsequently with iodine (FIG. 16B), wherein the collected fractions were run in lanes 1 and 2; an un-PEGylated sample of recombinant human erythropoietin was run in lane 3, a 30 kDa methoxy polyethylene glycol N-ethyl-(4-bromomethyl)-benzamide sample was run in lane 4 and molecular weight markers were run in lane 5;

As can be seen in FIGS. 16A-B (lane 3), EPO appeared as a 35 kDa band stained with Coomassie and not with iodine, and fraction 1 collected from RP-HPLC contains a mono-PEGylated product since it appeared as a band having a molecular weight of about 90 kDa (which is the sum of erythropoietin and the apparent molecular weight of PEG) in the Coomassie stained gel (FIG. 16A, lane 1), and was stained in brown with iodine (FIG. 16B, lane 1). Fraction 2 collected from RP-HPLC is the non-reacted erythropoietin (FIGS. 15A-B, lane 2) similarly to the erythropoietin standard (FIGS. 16A-B, lane 3).

These results indicate that a mono-PEGylated EPO was formed by using a PEG N-ethyl-(4-bromomethyl)-benzamide reagent at acidic conditions. In this case PEGylation may even be site-specific, since EPO has only one methionine.

### Example 12

### Peptide Mapping of PEGylated rh-EPO

In order to study the effect of PEGylation and to verify the PEGylation site along the polypeptide chain, PEGylated rh-erythropoietin (EPO) was subjected to protease digestion, and the breakup products were compared to those obtain from the un-PEGylated peptide.

Peptide mapping was performed according to Moya, G et al. [Biotecnologia Aplicada, 20, 214, 2003]. 120 μg of RECORMON® (30,000 IU/0.6 ml; an un-PEGylated form of rh-EPO by F. Hoffmann—La Roche, Ltd.) were concentrated and buffer exchanged to 100 mM Tris Acetate buffer pH 8.5 to a volume of 50 μl. Protein concentration (0.24 mg/ml) was determined using NANODROP® (by Thermo Fisher Scientific Inc.). Trypsin (2310 U/mg) treated with tosyl phenylalanyl chloromethyl ketone (TPCK) was prepared at a concentration of 1 mg/ml, and 0.5 mg/ml was added to 50 μl the RECORMON® preparation. Reaction was performed at 37° C. for 18 hours under mild rotation.

Peptide fragments were resolved by HPLC chromatography by injecting 50 μl of the mixture into a Jupiter C4 column heated to 30° C. with acetonitrile-water and 0.1% TFA as mobile phase.

Peptide mapping of PEGylated rh-EPO was performed simultaneously and in the same manner as peptide mapping of rh-EPO described above.

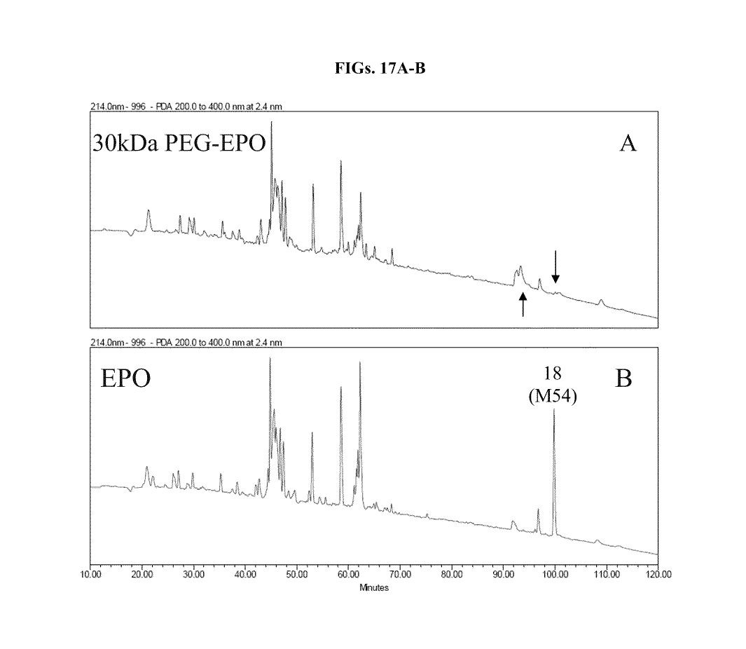

FIGS. 17A-B present RP-HPLC chromatograms comparing the peptide maps of purified PEGylation product of rh-EPO with 30 kDa methoxy polyethylene glycol N-ethyl-(4-bromomethyl)-benzamide (FIG. 17A), and of the un-PEGylated rh-EPO (FIG. 17B), as obtained by applying peptide digestion using trypsin.

As can be seen in FIG. 17B, the peptide map that was obtained by trypsin digestion of rh-EPO is similar to the published peptide maps of rh-EPO under similar conditions [Moya, G et al., Biotecnologia Aplicada, 20, 214, 2003 and Labrenz, S R et al., PDA J. Pharm. Sci. Technol., 62, 211, 2008], which allows assigning the peaks to the corresponding peptides.

As can further be seen in FIG. 17A, the peptide map of PEGylated rh-EPO digested by trypsin is similar to the peptide map of the un-PEGylated rh-EPO digested by the same protease. One makeable difference stems from the methionine residue in the peptide, which has almost totally disappeared from the digested PEGylated peptide map (FIG. 17A) as compared to the corresponding peak in rh-EPO digest map (FIG. 17B). In addition, in the case of the digested PEGylated protein, a new peptide was formed having a retention time of 94 minutes. Analysis by LC/MS/MS has shown that this new peak contains fragmentations of 44 Da which is typical fingerprint of the disrupted PEG chain.

From comparison of both peptide maps it is concluded that a site-specific PEGylation occurred via methionine amino acid M54.

### Example 13

### PEGylation of Recombinant Human Granulocyte Colony Stimulating Factor (rh G-CSF) with 30 kDa Methoxy Polyethylene Glycol N-ethyl-(4-bromomethyl)-benzamide

Granulocyte colony stimulating factor (G-CSF) stimulates the production of white blood cells, and therefore a recombinant form of G-CSF is used in oncology and hematology. rh G-CSF is used with certain cancer patients in treatment-resistant and/or metastatic breast cancer to prolong survival by accelerating recovery from neutropenia, allowing high-intensity chemotherapy regimens to be more sustained against myelosuppression and unacceptably-low levels of white blood cells. One of the most widely used recombinant human G-CSFs is synthesized in E. coli and is called Filgrastim (Neupogen®), which is structurally slightly different from the structure of the natural glycoprotein in terms of the post-translational saccharides modifications. Another form of recombinant human G-CSF is called Lenograstim, which is synthesized in Chinese Hamster Ovary cells (CHO cells), and since it is made in a mammalian cell expression system, it is indistinguishable from the 174-amino acid natural human G-CSF. There are no differences between Filgrastim and Lenograstim from the clinical or therapeutic point of view.

GCSF solution (0.3 mg/ml) was concentrated using Amicon Ultra 4 filter-10 kDa cut-off to a final concentration of about 4 mg/ml in Acetate buffer (10 mM, pH 4) with 5% mannitol. Final protein concentration was determined using Nanodrop spectrophotometer. 30 kDa methoxy polyethylene glycol N-ethyl-(4-bromomethyl)-benzamide (8 mg, 0.26 μmol) was added to the above solution of GCSF (1 mg, 0.073 μmol) and the reaction was incubated in a head-over-tail shaker at 25° C. for 24 hours.

Purification of the reaction mixture by preparative reverse-phase chromatography by loading 40 μl of the mixture on a Jupiter C4 column with acetonitrile-water and 0.2% TFA as mobile phase.

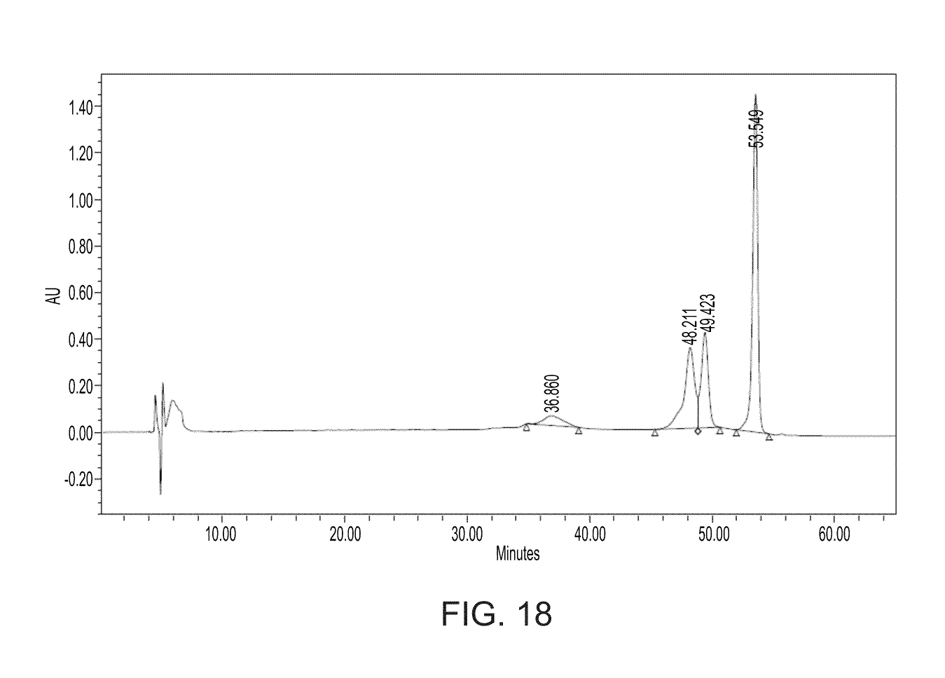

FIG. 18 presents a RP-HPLC chromatogram of the PEGylation reaction products of recombinant human granulocyte colony stimulating factor and 30 kDa methoxy polyethylene glycol N-ethyl-(4-bromomethyl)-benzamide wherein PEGylated protein peaks are observed with a retention time of 36.9, 48.2 and 49.4 minutes and recombinant human granulocyte colony stimulating factor has a retention time of 53.5 minutes. These fractions were concentrated to about ⅔ volume by speed-vac, and to avoid aggregation during further concentration, buffer was exchange to 10 mM acetate buffer, containing 5% mannitol, using Amicon Ultra 4 filters, and samples from these fractions were analyzed in a non-reducing SDS-PAGE.

The products from the reaction between GCSF and 30 kDa methoxy polyethylene glycol N-ethyl-(4-bromomethyl)-benzamide were collected by preparative reverse-phase chromatography.

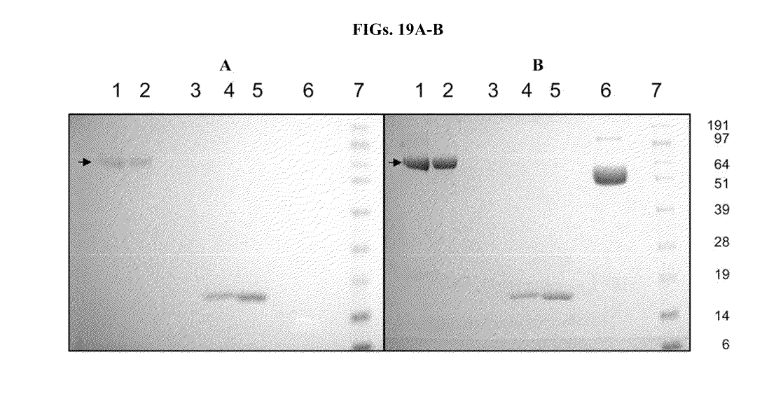

FIG. 19A-B present color images of a non-reducing SDS-PAGE separation gel in which the PEGylation reaction products of recombinant human granulocyte colony stimulating factor and 30 kDa methoxy polyethylene glycol N-ethyl-(4-bromomethyl)-benzamide, after collection by RP-HPLC, were run and stained with Coomassie Blue (FIG. 19A) and subsequently with iodine (FIG. 19B), wherein the collected fractions were run in lanes 1, 2, 3 and 4; an un-PEGylated sample of recombinant human granulocyte colony stimulating factor was run in lane 5, a 30 kDa methoxy polyethylene glycol N-ethyl-(4-bromomethyl)-benzamide sample was run in lane 6 and molecular weight markers were run in lane 7.

As can be seen in FIGS. 19A-B (lane 5), GCSF has an 18 kDa band stained with Coomassie and not with iodine. The PEG N-ethyl-(4-bromomethyl)-benzamide reagent stained only with iodine and not by the Coomassie blue, appeared as a brown band having an apparent molecular weight of about 55 kDa (FIG. 19B, lane 6). It can be observed that fraction 1 collected from RP-HPLC is a di-PEGylated product since it appeared as a band having a molecular weight of about 120 kDa (which is the sum of GCSF and twice the apparent molecular weight of PEG) in the Coomassie stained gel (FIG. 18A, lane 1), and was stained brown with iodine (FIG. 19B, lane 1). Fractions 2 and 3 are mainly mono-PEGylated products (mono-PEGylated isoforms) since they appeared as bands having a molecular weight of about 70 kDa (which is the sum of GCSF and the apparent molecular weight of PEG) in the Coomassie stained gel (FIG. 19A, lanes 2-3), and was stained brown with iodine (FIG. 19B, lanes 2-3). Fraction 4 collected from RP-HPLC is the non-reacted GCSF (FIGS. 19A-B, lane 4) similarly to the GCSF standard (FIGS. 19A-B, lane 5).

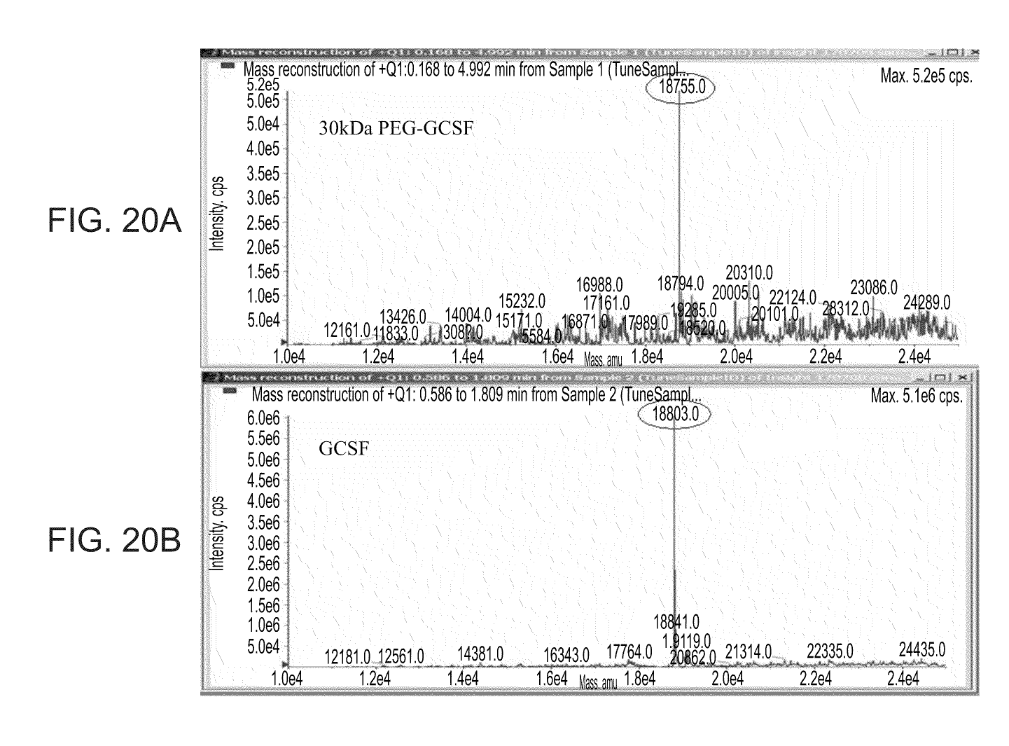

The 30 kDa PEGylated GCSF was also analyzed by electrospray ionization (ESI) mass spectrometry. FIGS. 20A-B present ESI mass spectrograms of the purified PEGylation product of recombinant human GCSF with 30 kDa methoxy polyethylene glycol N-ethyl-(4-bromomethyl)-benzamide (FIG. 20A) and recombinant human interferon-beta-1b (FIG. 20B).

As can be seen in FIGS. 20A-B, no molecular peak of a 30 kDa PEG-GCSF conjugate was observed (about 50 kDa) and the main peak was due to the Hoffman elimination described hereinabove, resulting in a mass of 18,755 Da which is exactly 48 Da less than the molecular mass of GCSF.

The results indicate that a mono-PEGylated GCSF was formed through a linkage with a methionine sulfur by using a PEG N-ethyl-(4-bromomethyl)-benzamide reagent at acidic conditions.

### Example 14

### PEGylation of Growth Hormone (GH) with 30 kDa Methoxy Polyethylene Glycol N-ethyl-(4-bromomethyl)-benzamide

Recombinant human Growth Hormone (rh-GH), marketed as Nutropin (Genentech), Humatrope (Lilly), Genotropin (Pfizer), Norditropin (Novo), and Saizen (Merck Serono) is produced by E. coli and mammalian cells. GH is used as replacement therapy in adults with GH deficiency of either childhood-onset (after completing growth phase) or adult-onset (usually as a result of an acquired pituitary tumor).

rh-GH (1 mg) was reconstituted by adding water (250 μl) to obtain a protein concentration of 4 mg/ml acetic acid (1 μl, 1:3) was added to the above solution (125 μl) reducing the pH to 4.

30 kDa methoxy polyethylene glycol N-ethyl-(4-bromomethyl)-benzamide (6.73 mg, 0. 22 μmol) was added to the above solution of GH (0.5 mg, 0.022 μmol) and the reaction was incubated in a head-over-tail shaker at 25° C. for 24 hours. Purification of the reaction mixture by preparative reverse-phase chromatography was performed by loading 15 μl of the mixture on a Jupiter C4 column with acetonitrile-water and 0.2% TFA as mobile phase. The fraction which was identified as the PEGylated protein was collected at retention time between 43-48 minutes. This fraction was concentrated by speed-vac and analyzed in a non-reducing SDS-PAGE.

The products from the reaction between GH and 30 kDa methoxy polyethylene glycol N-ethyl-(4-bromomethyl)-benzamide were collected by preparative reverse-phase chromatography.

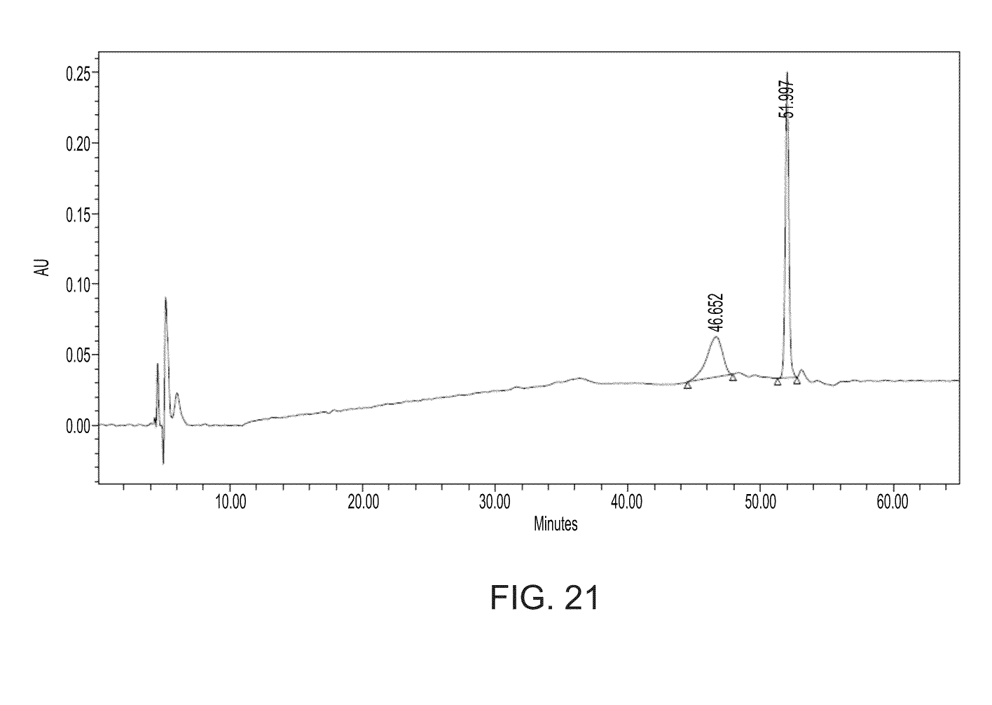

FIG. 21 presents a RP-HPLC chromatogram of the PEGylation reaction products of recombinant human growth hormone and 30 kDa methoxy polyethylene glycol N-ethyl-(4-bromomethyl)-benzamide wherein a new formed peak is observed with a retention time of 46.65 minutes and recombinant human growth hormone has a retention time of 52 minutes.

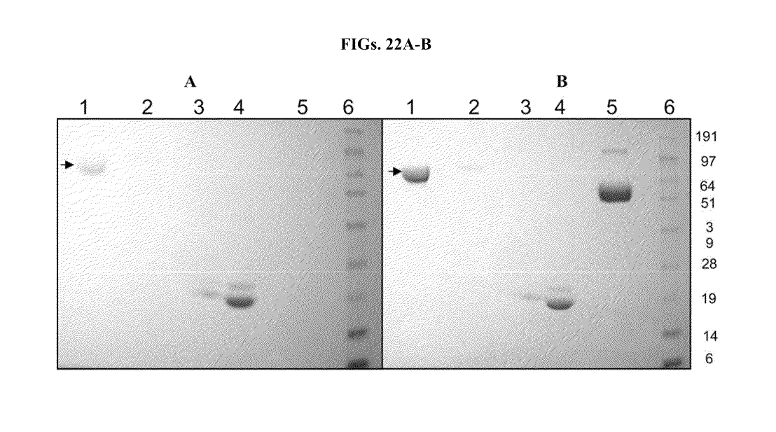

FIG. 22A-B present color images of a non-reducing SDS-PAGE separation gel in which the PEGylation reaction products of recombinant human growth hormone and 30 kDa methoxy polyethylene glycol N-ethyl-(4-bromomethyl)-benzamide, after collection by RP-HPLC, were run and stained with Coomassie Blue (FIG. 22A) and subsequently with iodine (FIG. 22B), wherein the collected fractions were run in lanes 1, 2, 3; an un-PEGylated sample of recombinant human growth hormone was run in lane 4, a 30 kDa methoxy polyethylene glycol N-ethyl-(4-bromomethyl)-benzamide sample was run in lane 5 and molecular weight markers were run in lane 6.

As can be seen in FIGS. 22A-B (lane 4), GH appeared as an 18 kDa band stained with Coomassie and not with iodine. The PEG N-ethyl-(4-bromomethyl)-benzamide reagent stained only with iodine and not by the Coomassie blue, appeared as a brown band having an apparent molecular weight of about 55 kDa (FIG. 22B, lane 5). It can be observed that fraction 1 collected from RP-HPLC is mainly a mono-PEGylated product since it appeared as bands having a molecular weight of about 70 kDa (which is the sum of GH and the apparent molecular weight of PEG) in the Coomassie stained gel (FIG. 22A, lane 1), and was stained brown with iodine (FIG. 22B, lane 1). Fraction 3 collected from RP-HPLC is the non-reacted GH (FIGS. 22A-B, lane 3) similarly to the GH standard (FIGS. 22A-B, lane 3).

The results indicate that a mono-PEGylated GH was formed by using a PEG N-ethyl-(4-bromomethyl)-benzamide reagent at acidic conditions.

### Example 15

### PEGylation of Follicle Stimulating Hormone (FSH) with 30 kDa Methoxy Polyethylene Glycol N-ethyl-(4-bromomethyl)-benzamide

Recombinant human Follicle Stimulating Hormone (rh-FSH), marketed as GONAL-f® (Merck Serono S. A.) and PUREGON® (Schering-Plough Corp.) is produced by mammalian cells.

Buffer of 100 μl of rh-FSH solution was exchanged to 20 mM acetate buffer pH 4 using MICROCON® 10K (Millipore) with 3 volume changes of 400 μL. Final protein concentration (2.17 mg/ml) was determined using NANODROP® spectrophotometer. 6 μl of a 20 mg/ml solution of 30 kDa methoxy polyethylene glycol N-ethyl-(4-bromomethyl)-benzamide (0.12 mg, 0.004 μmol) was added to 18.5 μl of the above solution of rh-FSH (0.04 mg, 0.002 μmol) and the reaction was incubated in a head-over-tail shaker at 25° C. for 17 hours. The reaction mixture was analyzed without further purification in a non-reducing SDS-PAGE. Another sample was prepared immediately before running a non-reducing SDS-PAGE as time zero reference.

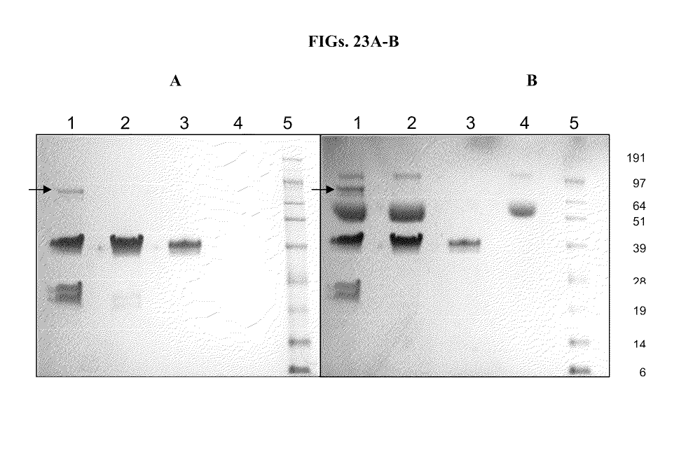

FIGS. 23A-B present color images of a non-reducing SDS-PAGE separation gel in which the PEGylation reaction products of recombinant human follicle stimulating hormone and 30 kDa methoxy polyethylene glycol N-ethyl-(4-bromomethyl)-benzamide were run and stained with Coomassie Blue (FIG. 23A) and subsequently with iodine (FIG. 23B), wherein the reaction mixture after 17 hrs. was run in lane 1; the reaction mixture at time zero was run in lane 2; an un-PEGylated sample of recombinant human follicle stimulating hormone was run in lane 3, a 30 kDa methoxy polyethylene glycol N-ethyl-(4-bromomethyl)-benzamide sample was run in lane 4 and molecular weight markers were run in lane 5.

As can be seen in FIGS. 23A-B, rh-FSH appeared as an 35 kDa band stained with Coomassie and not with iodine (lane 3). The PEG N-ethyl-(4-bromomethyl)-benzamide reagent stained only with iodine and not by the Coomassie blue, appeared as a brown band having an apparent molecular weight of about 55 kDa (FIG. 23B, lane 4). It can be observed that a new product was formed since a new band appeared in the reaction mixture after 17 hours (lane 1) which did not exist at time zero (lane 2). The newly formed product is mono-PEGylated rh-FSH since it appeared as bands having a molecular weight of about 90 kDa, which is the sum of the apparent molecular weights of rh-FSH and PEG, in the Coomassie stained gel (FIG. 23A, lane 1), and was stained brown with iodine (FIG. 23B, lane 1).

These results indicate that a mono-PEGylated rh-FSH was formed by using only 2 equivalents of PEG N-ethyl-(4-bromomethyl)-benzamide reagent at acidic conditions.

### Example 16

### Attachment of a Fluorescent Probe to Methionine Containing Proteins

Lucifer yellow is a well-known polar tracer for neurons. Its iodoacetamide derivative has high water solubility and visible absorption and emission similar to those of Lucifer yellow.

A methionine-containing protein is reacted with an iodoacetamide derivative of Lucifer yellow, at an acidic pH, using the procedure described in Example 2 hereinabove, as depicted in Scheme 4 below.

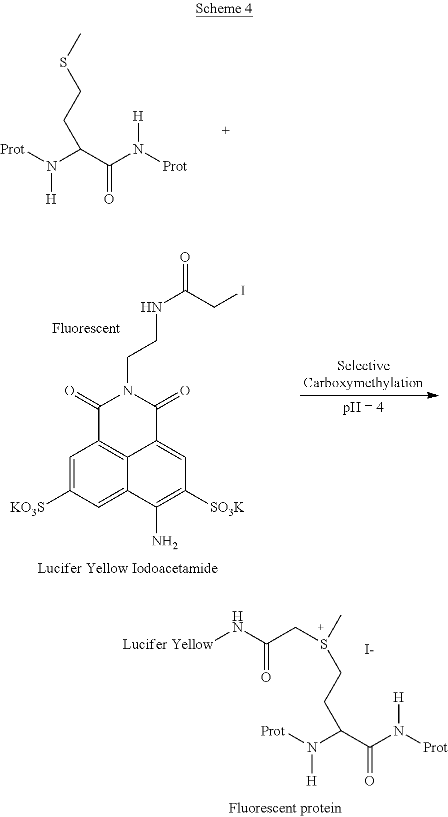

### Example 17

### Two-Steps PEGylation of Methionine Containing Proteins

A two-step PEGylation of recombinant and/or native proteins which exhibit at least one unmodified methionine side chain in their structure can be performed in cases wherein the methionine side-chain(s) is less accessible.

Thus, the protein is modified with a modifying moiety having three basic components, a first and second reactive moieties and a linking moiety which can be a spacer. The protein is modified at a methionine side-chain as illustrated in Scheme 5 hereinbelow.

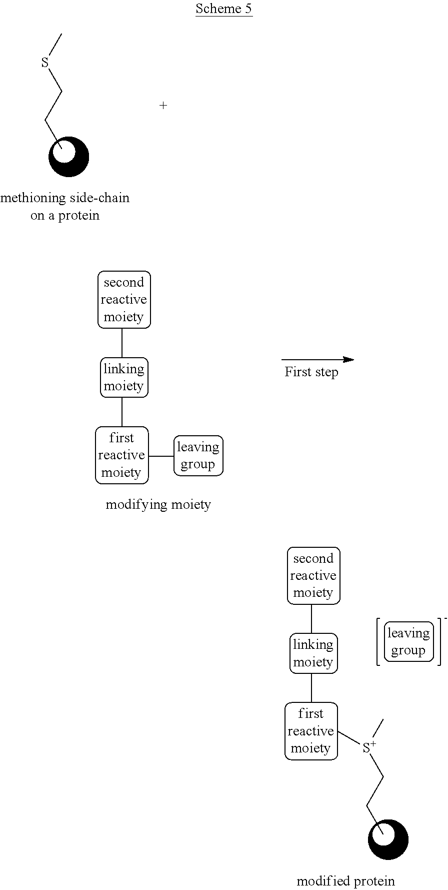

This reaction is conducted under conditions that favor a formation of a covalent bond between the first reactive moiety and the sulfur atom of the methionine side-chain, and according to some embodiments, under acidic conditions.

The modified protein is then reacted with a PEG moiety having a third reactive group which is capable of forming a covalent bond with the second reactive group, as illustrated in Scheme 6 hereinbelow.

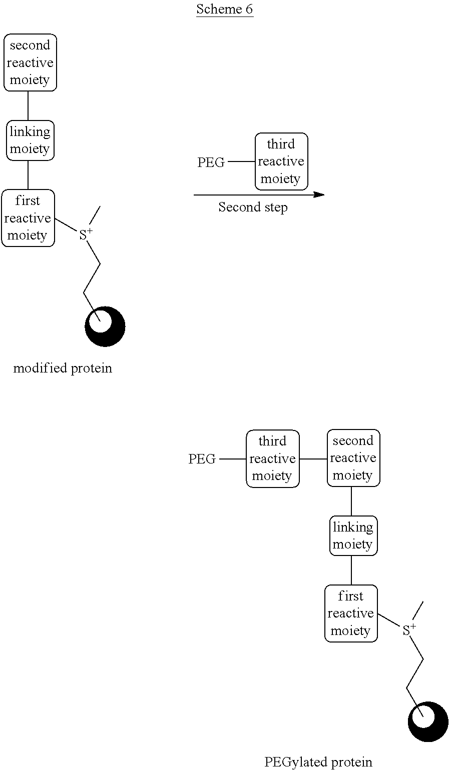

In a typical example, a methionine residue is reacted with a reactive derivative of an alkylacetylhydrazide to form a methionine sulfonium moiety having a substituent termination with an acetylhydrazide. The hydrazide group which becomes exposed to the surface is then selectively modified at low pH with high MW PEG-aldehyde by reductive alkylation, as depicted in Scheme 7.

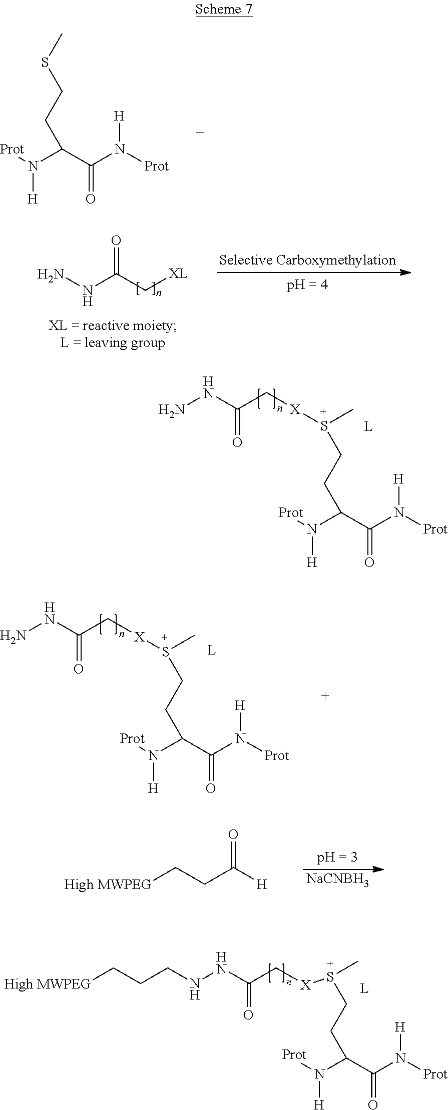

### Example 18

Using the General procedure described hereinabove, a wide variety of proteins, and particularly proteins of therapeutic and pharmacological importance can be PEGylated owing to the fact that they have at least one methionine residue within their sequence.

Exemplary protein families which can be PEGylated using the abovementioned general procedure include human blood factors, human hormones, human growth factors and cytokines, enzymes, antibodies and fusion proteins.

Representative examples of human blood factors which can be PEGylated according to the present embodiments include recombinant human Factor VIII, recombinant human B-deleted domain Factor VIII, recombinant human Factor VIIa, recombinant human Factor IX, recombinant human tissue plasminogen activator (TPA), recombinant human activated Protein C and recombinant human thrombin.

Representative examples of human hormones which can be PEGylated according to the present embodiments include recombinant human growth hormone (GH), recombinant human follicle stimulating hormone (FSH), recombinant human luteinizing hormone (LH), recombinant human parathyroid hormone (PTH), recombinant human parathyroid hormone (1-34) (PTH 1-34), recombinant human chorionic gonadotropin (CG), recombinant human thyrotropin (TSH) and recombinant human glucagons.

Representative examples of human growth factors and cytokines which can be PEGylated according to the present embodiments include recombinant human erythropoietin (EPO), recombinant human thrombopoietin (TPO), recombinant human granulocyte-macrophage colony stimulating factor (GM-CSF), recombinant human granulocyte colony stimulating factor (G-CSF), recombinant human insulin-like growth factor-1 (IGF-1), recombinant human keratinocyte growth factor (KGF), recombinant human platelet-derived growth factor (PDGF), recombinant human bone morphogenetic protein-2 (BMP-2), recombinant human bone morphogenetic protein-7 (BMP-7), recombinant human tumor necrosis factor-alpha (TNF-alpha), recombinant human interferon-alpha-2a (IFN_alpha-2a), recombinant human interferon-alpha-2b (IFN_alpha-2b), recombinant human interferon-gamma-1b (IFN_gamma-1b), recombinant human interleukin-1 (IL-1) receptor antagonist, recombinant human interleukin (IL-2) and recombinant human interleukin (IL-11).

Representative examples of enzymes which can be PEGylated according to the present embodiments include recombinant human heparanase, recombinant human alglucosidase-alpha, recombinant human imiglucerase, recombinant human laronidase, recombinant human agalsidase-beta, recombinant human galsulfase, recombinant human hyaluronidase, recombinant human alpha-galactosidase, recombinant urate oxidase and recombinant human dornase-alpha.

Representative examples of antibodies which can be PEGylated according to the present embodiments include recombinant human rituximab, recombinant human trastuzumab and recombinant human cetuximab.

Representative examples of fusion proteins which can be PEGylated according to the present embodiments include etanercept, alefacept and r-IL-2 diphteria toxin fusion protein.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-45. (canceled)

46. A conjugate comprising:
(a) a polypeptide having at least one methionine residue, each said methionine residue having a methylsulfanyl-ethyl side-chain; and
(b) at least one polymer moiety being covalently attached to a sulfur atom of said methylsulfanyl-ethyl side-chain of at least one said methionine residue.

47. The conjugate of claim 46, wherein said at least one polymer moiety is covalently attached to said sulfur atom via a linking moiety.

48. The conjugate of claim 47, wherein said linking moiety comprises at least one residue of a reactive moiety, said reactive moiety being selected capable of reacting with said sulfur atom of said methylsulfanyl-ethyl side-chain and said residue of said reactive moiety being formed upon said reacting.

49. The conjugate of claim 48, wherein said reactive moiety is selected from the group consisting of amine, carboxyl, amide, acetamide, 2-halo-acetamide, (4-halomethyl)-benzamide, benzyl-halide, hydrazine, hydrazide, acetohydrazide, alkyl, haloalkyl, alkyl sulfonylhalide, alkyl tosylate, alkyl triflate, allyl, haloallyl, allyl sulfonylhalide, allyl tosylate, allyl triflate, aryl, haloaryl heteroaryl, 4-(halomethyl)benzyl, benzyl and halobenzyl, and any combination thereof.

50. The conjugate of claim 48, wherein said reactive moiety is selected from the group consisting of 2-halo-acetamide, (4-halomethyl)-benzamide, benzyl-halide, haloalkyl, hydrazine, hydrazide and acetohydrazide.

51. The conjugate of claim 48, wherein said reactive moiety comprises at least one leaving group selected from the group consisting of halide, acetate, tosylate, triflate, sulfonate, azide, hydroxy, thiohydroxy, alkoxy, cyanate, thiocyanate, nitro and cyano.

52. The conjugate of claim 47, wherein said linking moiety further comprises a spacer.

53. The conjugate of claim 52, wherein said spacer is selected from the group consisting of methane-di-yl, ethane-1-yl-2-yl, propane-1-yl-3-yl, butane-1-yl-4-yl, 1,4-benzene-diyl and 1,10-biphenyl-diyl.

54. The conjugate of claim 46, wherein said polypeptide is selected from the group consisting of an interferon, a cytokine, a hormone, a growth factor, an enzyme, a blood protein (factor), an antibody, an antigen, a viral protein, a fusion protein, and any part or segment thereof.

55. The conjugate of claim 46, wherein said polypeptide is selected from the group consisting of adalimumab, adenosine deaminase, agalsidase-beta, alglucosidase-alpha, alpha-galactosidase, asparaginase, B-deleted domainFactor VIII, bone morphogenetic protein-2 (BMP-2), bone morphogenetic protein-7 (BMP-7), brain-derived neurotrophic factor (BDNF), cetuximab, chorionic gonadotropin (CG), dornase-alpha, erythropoietin (EPO), etanercept, Factor IX, Factor VIIa, Factor VIII, follicle stimulating hormone (FSH), galsulfase, glial cell line derived neurotrophic factor (GDNF), glucagon, granulocyte colony stimulating factor (G-CSF), granulocyte-macrophage colony stimulating factor (GM-CSF), a growth hormone (GH), hemoglobin, heparanase, hyaluronidase, imiglucerase, infliximab, insulin-like growth factor-1 (IGF-1), interferon-alpha-2a (IFN alpha-2a), interferon-alpha-2b (IFN alpha-2b), interferon-beta-1a (IFN beta-1a), interferon-beta-1b (IFN beta-1b), interferon-gamma-1b (IFN gamma-1b), interleukin (IL-11), interleukin (IL-2), interleukin-1 (IL-1) receptor antagonist, interleukin-1 receptor antagonist (IL-1ra), keratinocyte growth factor (KGF), laronidase, luteinizing hormone (LH), megakaryocyte growth differentiation factor (MGDF), obesity protein (OB protein or leptin), osteoprotegerin (OPG), parathyroid hormone (PTH or 1-34 segment or PTH 1-34), palivizumab, platelet-derived growth factor (PDGF), Protein C, rituximab, stem cell factor (SCF), streptokinase, thrombin, thrombopoietin (TPO), thyrotropin (TSH), tissue plasminogen activator (tPA), trastuzumab, tumor necrosis factor binding protein (TNFbp), tumor necrosis factor-alpha (TNF-alpha) and urate-oxidase, and any part or segment thereof.

56. The conjugate of claim 46, wherein said polypeptide is selected from the group consisting of interferon-alpha-2a (INF-α2a), interferon-beta-1a (INF-β1a), interferon-beta-1b (INF-β1b), erythropoietin (EPO), granulocyte colony-stimulating factor (G-CSF) human growth hormone (h-GH).

57. The conjugate of claim 46, wherein said polymer moiety is selected from the group consisting of a polyalkylene glycol, a polyethylene glycol (PEG), a poly(lactic acid) (PLA), a polyester, a polyglycolide (PGA), a polycaprolactone (PCL), a polyamide, a polymethacrylamide, a polyvinyl alcohol, a polycarboxylate, a polyvinyl pyrrolidinone, a dextran, a cellulose, a chitosan, a hydroxyethyl starch (HES), polyglutamic acid, polyglycine and any copolymer thereof.

58. The conjugate of claim 46, wherein said polymer moiety is a polyethylene glycol (PEG).

59. The conjugate of claim 58, wherein said polyethylene glycol has an average molecular weight that ranges from 4 kDa to 40 kDa.

60. The conjugate of claim 46, being selected from the group consisting of:
a conjugate comprising interferon-beta-1b and 30 kDa methoxy polyethylene glycol N-ethyl-acetamide, being attached therebetween via a sulfur atom of a methionine residue of said interferon-beta-1b, and having a formula: 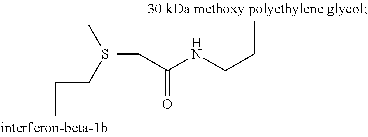
a conjugate comprising interferon-beta-1b and 30 kDa methoxy polyethylene glycol N-ethyl-(4-bromomethyl)-benzamide, being attached therebetween via a sulfur atom of a methionine residue of said interferon-beta-1b, and having a formula: 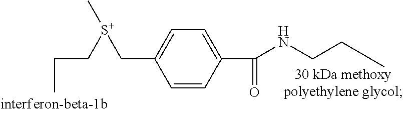
a conjugate comprising interferon-beta-1a and 30 kDa methoxy polyethylene glycol N-ethyl-acetamide, being attached therebetween via a sulfur atom of a methionine residue of said interferon-beta-1a, and having a formula: 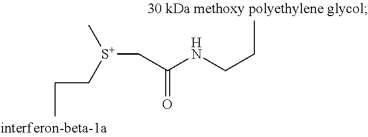
a conjugate comprising interferon-alpha-2a and 30 kDa methoxy polyethylene glycol N-ethyl-acetamide, being attached therebetween via a sulfur atom of a methionine residue of said interferon-alpha-2a, and having a formula: 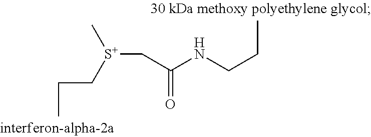
a conjugate comprising interferon-alpha-2a and 30 kDa methoxy polyethylene glycol N-ethyl-(4-bromomethyl)-benzamide, being attached therebetween via a sulfur atom of a methionine residue of said interferon-alpha-2a, and having a formula: 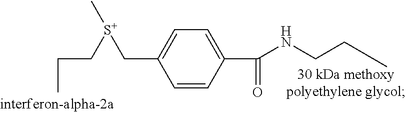
a conjugate comprising erythropoietin and 30 kDa methoxy polyethylene glycol N-ethyl-(4-bromomethyl)-benzamide, being attached therebetween via a sulfur atom of a methionine residue of said erythropoietin, and having a formula: 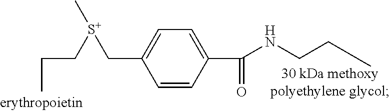
a conjugate comprising granulocyte colony-stimulating factor (G-CSF) and 30 kDa methoxy polyethylene glycol N-ethyl-(4-bromomethyl)-benzamide, being attached therebetween via a sulfur atom of a methionine residue of said G-CSF, and having a formula: 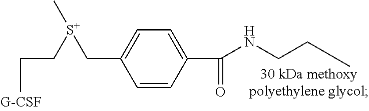
a conjugate comprising human growth hormone (h-GH) and 30 kDa methoxy polyethylene glycol N-ethyl-(4-bromomethyl)-benzamide, being attached therebetween via a sulfur atom of a methionine residue of said h-GH, and having a formula: 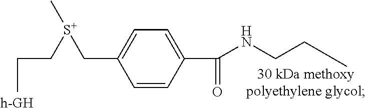
and
a conjugate comprising human follicle stimulating hormone (h-FSH) and 30 kDa methoxy polyethylene glycol N-ethyl-(4-bromomethyl)-benzamide, being attached therebetween via a sulfur atom of a methionine residue of said h-FSH, and having a formula:

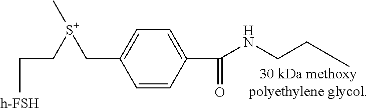

61. A process of preparing the conjugate of claim 46, the process comprising:
reacting said polypeptide with said polymer having at least one reactive moiety attached thereto, under acidic conditions ranging from about pH of 2 to pH of 5, said reactive moiety being selected capable of reacting with said sulfur atom in said methylsulfanyl-ethyl side-chain, thereby obtaining the conjugate.

62. A pharmaceutical composition comprising the conjugate of claim 46.

63. The pharmaceutical composition of claim 62, wherein said polypeptide is selected from the group consisting of an interferon, a cytokine, a hormone, a growth factor, an enzyme, a blood protein (factor), an antibody, an antigen, a viral protein, a fusion protein, and any part or segment thereof.

64. The pharmaceutical composition of claim 62, wherein said polypeptide is selected from the group consisting of adalimumab, adenosine deaminase, agalsidase-beta, alglucosidase-alpha, alpha-galactosidase, asparaginase, B-deleted domainFactor VIII, bone morphogenetic protein-2 (BMP-2), bone morphogenetic protein-7 (BMP-7), brain-derived neurotrophic factor (BDNF), cetuximab, chorionic gonadotropin (CG), dornase-alpha, erythropoietin (EPO), etanercept, Factor IX, Factor VIIa, Factor VIII, follicle stimulating hormone (FSH), galsulfase, glial cell line derived neurotrophic factor (GDNF), glucagon, granulocyte colony stimulating factor (G-CSF), granulocyte-macrophage colony stimulating factor (GM-CSF), a growth hormone (GH), hemoglobin, heparanase, hyaluronidase, imiglucerase, infliximab, insulin-like growth factor-1 (IGF-1), interferon-alpha-2a (IFN alpha-2a), interferon-alpha-2b (IFN alpha-2b), interferon-beta-1a (IFN beta-1a), interferon-beta-1b (IFN beta-1b), interferon-gamma-1b (IFN gamma-1b), interleukin (IL-11), interleukin (IL-2), interleukin-1 (IL-1) receptor antagonist, interleukin-1 receptor antagonist (IL-1ra), keratinocyte growth factor (KGF), laronidase, luteinizing hormone (LH), megakaryocyte growth differentiation factor (MGDF), obesity protein (OB protein or leptin), osteoprotegerin (OPG), parathyroid hormone (PTH or 1-34 segment or PTH 1-34), palivizumab, platelet-derived growth factor (PDGF), Protein C, rituximab, stem cell factor (SCF), streptokinase, thrombin, thrombopoietin (TPO), thyrotropin (TSH), tissue plasminogen activator (tPA), trastuzumab, tumor necrosis factor binding protein (TNFbp), tumor necrosis factor-alpha (TNF-alpha) and urate-oxidase, and any part or segment thereof.

65. The pharmaceutical composition of claim 62, wherein said polypeptide is selected from the group consisting of interferon-alpha-2a (INF-α2a), interferon-beta-1a (INF-β1a), interferon-beta-1b (INF-β1b), erythropoietin (EPO), granulocyte colony-stimulating factor (G-CSF) human growth hormone (h-GH).

66. The pharmaceutical composition of claim 62, wherein said polymer moiety is selected from the group consisting of a polyalkylene glycol, a polyethylene glycol (PEG), a poly(lactic acid) (PLA), a polyester, a polyglycolide (PGA), a polycaprolactone (PCL), a polyamide, a polymethacrylamide, a polyvinyl alcohol, a polycarboxylate, a polyvinyl pyrrolidinone, a dextran, a cellulose, a chitosan, a hydroxyethyl starch (HES), polyglutamic acid, polyglycine and any copolymer thereof.

67. The pharmaceutical composition of claim 62, wherein said polymer moiety is a polyethylene glycol (PEG).

68. The pharmaceutical composition of claim 67, wherein said polyethylene glycol has an average molecular weight that ranges from 4 kDa to 40 kDa.

69. A method of treating a medical condition treatable by a polypeptide having at least one methionine residue, the method comprising administering to a subject in need thereof an therapeutically effective amount of the conjugate of claim 46.

70. The method of claim 69, wherein said polypeptide is selected from the group consisting of an interferon, a cytokine, a hormone, a growth factor, an enzyme, a blood protein (factor), an antibody, an antigen, a viral protein, a fusion protein, and any part or segment thereof.

71. The method of claim 69, wherein said polypeptide is selected from the group consisting of adalimumab, adenosine deaminase, agalsidase-beta, alglucosidase-alpha, alpha-galactosidase, asparaginase, B-deleted domainFactor VIII, bone morphogenetic protein-2 (BMP-2), bone morphogenetic protein-7 (BMP-7), brain-derived neurotrophic factor (BDNF), cetuximab, chorionic gonadotropin (CG), dornase-alpha, erythropoietin (EPO), etanercept, Factor IX, Factor VIIa, Factor VIII, follicle stimulating hormone (FSH), galsulfase, glial cell line derived neurotrophic factor (GDNF), glucagon, granulocyte colony stimulating factor (G-CSF), granulocyte-macrophage colony stimulating factor (GM-CSF), a growth hormone (GH), hemoglobin, heparanase, hyaluronidase, imiglucerase, infliximab, insulin-like growth factor-1 (IGF-1), interferon-alpha-2a (IFN alpha-2a), interferon-alpha-2b (IFN alpha-2b), interferon-beta-1a (IFN beta-1a), interferon-beta-1b (IFN beta-1b), interferon-gamma-1b (IFN gamma-1b), interleukin (IL-11), interleukin (IL-2), interleukin-1 (IL-1) receptor antagonist, interleukin-1 receptor antagonist (IL-1ra), keratinocyte growth factor (KGF), laronidase, luteinizing hormone (LH), megakaryocyte growth differentiation factor (MGDF), obesity protein (OB protein or leptin), osteoprotegerin (OPG), parathyroid hormone (PTH or 1-34 segment or PTH 1-34), palivizumab, platelet-derived growth factor (PDGF), Protein C, rituximab, stem cell factor (SCF), streptokinase, thrombin, thrombopoietin (TPO), thyrotropin (TSH), tissue plasminogen activator (tPA), trastuzumab, tumor necrosis factor binding protein (TNFbp), tumor necrosis factor-alpha (TNF-alpha) and urate-oxidase, and any part or segment thereof.

72. The method of claim 69, wherein said polypeptide is selected from the group consisting of interferon-alpha-2a (INF-α2a), interferon-beta-1a (INF-β1a), interferon-beta-1b (INF-β1b), erythropoietin (EPO), granulocyte colony-stimulating factor (G-CSF) human growth hormone (h-GH).

73. The method of claim 69, wherein said polymer moiety is selected from the group consisting of a polyalkylene glycol, a polyethylene glycol (PEG), a poly(lactic acid) (PLA), a polyester, a polyglycolide (PGA), a polycaprolactone (PCL), a polyamide, a polymethacrylamide, a polyvinyl alcohol, a polycarboxylate, a polyvinyl pyrrolidinone, a dextran, a cellulose, a chitosan, a hydroxyethyl starch (HES), polyglutamic acid, polyglycine and any copolymer thereof.

74. The method of claim 69, wherein said polymer moiety is a polyethylene glycol (PEG).

75. A compound comprising:
(a) a polypeptide having at least one methionine residue, each said methionine residue having a methylsulfanyl-ethyl side-chain; and
(b) at least one modifying moiety which comprises a residue of a first reactive moiety and a second reactive moiety, said modifying moiety being covalently attached to a sulfur atom of said methylsulfanyl-ethyl side-chain of at least one said methionine residue via said residue of said first reactive moiety,
said polypeptide being selected from the group consisting of adalimumab, adenosine deaminase, agalsidase-beta, alglucosidase-alpha, alpha-galactosidase, asparaginase, B-deleted domainFactor VIII, bone morphogenetic protein-2 (BMP-2), bone morphogenetic protein-7 (BMP-7), brain-derived neurotrophic factor (BDNF), cetuximab, chorionic gonadotropin (CG), dornase-alpha, erythropoietin (EPO), etanercept, Factor IX, Factor VIIa, Factor VIII, follicle stimulating hormone (FSH), galsulfase, glial cell line derived neurotrophic factor (GDNF), glucagon, granulocyte colony stimulating factor (G-CSF), granulocyte-macrophage colony stimulating factor (GM-CSF), growth hormone (GH), hemoglobin, heparanase, hyaluronidase, imiglucerase, infliximab, insulin-like growth factor-1 (IGF-1), interferon-alpha-2a (IFN alpha-2a), interferon-alpha-2b (IFN alpha-2b), interferon-beta-1a (IFN beta-1a), interferon-beta-1b (IFN beta-1b), interferon-gamma-1b (IFN gamma-1b), interleukin (IL-11), interleukin (IL-2), interleukin-1 (IL-1) receptor antagonist, interleukin-1 receptor antagonist (IL-1ra), keratinocyte growth factor (KGF), laronidase, luteinizing hormone (LH), megakaryocyte growth differentiation factor (MGDF), obesity protein (OB protein or leptin), osteoprotegerin (OPG), parathyroid hormone (PTH or 1-34 segment or PTH 1-34), palivizumab, platelet-derived growth factor (PDGF), Protein C, rituximab, stem cell factor (SCF), streptokinase, thrombin, thrombopoietin (TPO), thyrotropin (TSH), tissue plasminogen activator (tPA), trastuzumab, tumor necrosis factor binding protein (TNFbp), tumor necrosis factor-alpha (TNF-alpha) and urate-oxidase.

76. The compound of claim 75, wherein said polypeptide is selected from the group consisting of interferon-alpha-2a (INF-α2a), interferon-beta-1a (INF-β1a), interferon-beta-1b (INF-β1b), erythropoietin (EPO), granulocyte colony-stimulating factor (G-CSF), human growth hormone (h-GH) and follicle stimulating hormone (FSH).

77. The compound of claim 75, wherein said first reactive moiety is capable of reacting with said sulfur atom and is selected from the group consisting of amine, carboxyl, amide, acetamide, 2-halo-acetamide, (4-halomethyl)-benzamide, benzyl-halide, hydrazine, hydrazide, acetohydrazide, alkyl, haloalkyl, alkyl sulfonylhalide, alkyl tosylate, alkyl triflate, allyl, haloallyl, allyl sulfonylhalide, allyl tosylate, allyl triflate, aryl, haloaryl heteroaryl, 4-(halomethyl)benzyl, benzyl and halobenzyl, and any combination thereof.

78. The compound of claim 75, wherein said second reactive moiety is selected from the group consisting of amine, carboxyl, amide, hydrazine, hydrazide, thiol, hydroxyl and hydroxylamine, and any combination thereof.

79. The compound of claim 77, wherein said modifying moiety further comprises a spacer connecting said residue of said first reactive moiety and said second reactive moiety.

80. The compound of claim 75, further comprising a labeling moiety being covalently attached to said modifying moiety.

81. A process of preparing the compound of claim 75, the process comprising:
reacting said polypeptide with a modifying moiety having a first reactive moiety and a second reactive moiety under acidic conditions ranging from pH 2 to pH 5, said first and second reactive moieties are selected such that a covalent bond is formed between said first reactive group and said sulfur atom, thereby obtaining the compound.

82. A process of preparing a conjugate which comprises:
a) a polypeptide having at least one methionine residue; and
b) at least one polymer moiety attached to a sulfur atom of a methylsulfanyl-ethyl side-chain of said at least one methionine residue;
the process comprising:
reacting said polypeptide with at least one modifying moiety having a first reactive moiety and a second reactive moiety under acidic conditions ranging from pH 2 to pH 5, said first and second reactive moieties are selected such that a covalent bond is formed between said first reactive moiety and said sulfur atom, to thereby obtain a polypeptide having said at least one modifying moiety attached thereto; and
reacting said polypeptide having said at least one modifying moiety attached thereto with a polymer having a third reactive moiety, said third reactive moiety is selected capable of reacting with said second reactive moiety in said modifying moiety, thereby obtaining the conjugate.

83. The process of claim 82, wherein said polypeptide is selected from the group consisting of interferon-alpha-2a (INF-α2a), interferon-beta-1a (INF-β1a), interferon-beta-1b (INF-β1b), erythropoietin (EPO), granulocyte colony-stimulating factor (G-CSF), a growth hormone (GH) and follicle stimulating hormone (FSH).

84. The process of claim 82, wherein said first reactive moiety is selected from the group consisting of amine, carboxyl, amide, acetamide, hydrazine, hydrazide, acetohydrazide, alkyl, haloalkyl, alkyl sulfonylhalide, alkyl tosylate, alkyl triflate, allyl, haloallyl, allyl sulfonylhalide, allyl tosylate, allyl triflate, aryl, haloaryl heteroaryl, 4-(halomethyl)benzyl, 4-(halomethyl)benzamide, benzyl and halobenzyl, and any combination thereof.

85. The process of claim 82, wherein said second and said third reactive moieties are each independently selected from the group consisting of amine, carboxyl, amide, hydrazine, hydrazide, thiol, hydroxyl and hydroxylamine, and any combination thereof.

86. The process of claim 82, wherein said polymer moiety is selected from the group consisting of a polyalkylene glycol, a polyethylene glycol (PEG), a poly(lactic acid) (PLA), a polyester, a polyglycolide (PGA), a polycaprolactone (PCL), a polyamide, a polymethacrylamide, a polyvinyl alcohol, a polycarboxylate, a polyvinyl pyrrolidinone, a dextran, a cellulose, a chitosan, a hydroxyethyl starch (HES), polyglutamic acid, polyglycine and any copolymer thereof.

87. The process of claim 82, wherein said polymer moiety is a polyethylene glycol (PEG).

88. A compound comprising a polyalkylene glycol moiety and a benzyl halide moiety being covalently linked therebetween via a linking moiety, said polyalkylene glycol moiety having an average molecular weight that ranges from 20 kDa to 40 kDa.
